SEQUENCE LISTING (34 sequences)

SEQ ID NO: 1 - DNA, 21 bp, Artificial Sequence
  Synthetic sequence
aaatgttaacgggaggctgcc

SEQ ID NO: 2 - DNA, 21 bp, Artificial Sequence
  Synthetic sequence
ttcctgctccaatgatggtgc

SEQ ID NO: 3 - DNA, 21 bp, Artificial Sequence
  Synthetic sequence
ttcgacttggatgccctgttg

SEQ ID NO: 4 - DNA, 22 bp, Artificial Sequence
  Synthetic sequence
ctttctgggagatggccaattg

SEQ ID NO: 5 - DNA, 21 bp, Artificial Sequence
  Synthetic sequence
catcatcgtggagaagccctt

SEQ ID NO: 6 - DNA, 22 bp, Artificial Sequence
  Synthetic sequence
gatcctgttggcaaatctcagc

SEQ ID NO: 7 - DNA, 22 bp, Artificial Sequence
  Synthetic sequence
agagcaggagcaacgtgaaatc

SEQ ID NO: 8 - DNA, 22 bp, Artificial Sequence
  Synthetic sequence
agagcctccatgtagaatggca

SEQ ID NO: 9 - DNA, 22 bp, Artificial Sequence
  Synthetic sequence
cgacggaaagagtatgagctgg

SEQ ID NO: 10 - DNA, 22 bp, Artificial Sequence
  Synthetic sequence
ctgggagtagttggcagatcca

SEQ ID NO: 11 - DNA, 21 bp, Artificial Sequence
  Synthetic sequence
gtgctgggtccaattcacaag

SEQ ID NO: 12 - DNA, 20 bp, Artificial Sequence
  Synthetic sequence
caccccggtagttgggagta

SEQ ID NO: 13 - DNA, 22 bp, Artificial Sequence
  Synthetic sequence
gctgtctccaggtgacattcca

SEQ ID NO: 14 - DNA, 22 bp, Artificial Sequence
  Synthetic sequence
cgctcctcccgagttctatcat

SEQ ID NO: 15 - DNA, 22 bp, Artificial Sequence
  Synthetic sequence
atgctgaagtgtggcttcctgt

SEQ ID NO: 16 - DNA, 22 bp, Artificial Sequence
  Synthetic sequence
tctgcagtctctttggccttgt

SEQ ID NO: 17 - DNA, 21 bp, Artificial Sequence
  Synthetic sequence
ccggacaccatggacaagttt

SEQ ID NO: 18 - DNA, 22 bp, Artificial Sequence
  Synthetic sequence
gtgggcaaggtgctattgaaag

SEQ ID NO: 19 - DNA, 20 bp, Artificial Sequence
  Synthetic sequence
ttatgcctgccaaagcgaca

SEQ ID NO: 20 - DNA, 22 bp, Artificial Sequence
  Synthetic sequence
atcccacacttccatttgtccc

SEQ ID NO: 21 - DNA, 21 bp, Artificial Sequence
  Synthetic sequence
catcatcgtggagaagccctt

SEQ ID NO: 22 - DNA, 22 bp, Artificial Sequence
  Synthetic sequence
gatcctgttggcaaatctcagc

SEQ ID NO: 23 - DNA, 22 bp, Artificial Sequence
  Synthetic sequence
gggtatctttccaggcttccct

SEQ ID NO: 24 - DNA, 22 bp, Artificial Sequence
  Synthetic sequence
ttccctaagtggcctcttgagc

SEQ ID NO: 25 - DNA, 22 bp, Artificial Sequence
  Synthetic sequence
tggagtcctattgacatcgcca

SEQ ID NO: 26 - DNA, 20 bp, Artificial Sequence
  Synthetic sequence
ccgcccaaagggaactgata

SEQ ID NO: 27 - DNA, 22 bp, Artificial Sequence
  Synthetic sequence
ttccttaccgtgggcactgaag

SEQ ID NO: 28 - DNA, 22 bp, Artificial Sequence
  Synthetic sequence
gaactgaggatgctgaagggca

SEQ ID NO: 29 - DNA, 22 bp, Artificial Sequence
  Synthetic sequence
gcatccaccacaacagtgtgga

SEQ ID NO: 30 - DNA, 22 bp, Artificial Sequence
  Synthetic sequence
cattcgccactcgttcctctct

SEQ ID NO: 31 - DNA, 21 bp, Artificial Sequence
  Synthetic sequence
caaatctggaacccgtgtgga

SEQ ID NO: 32 - DNA, 21 bp, Artificial Sequence
  Synthetic sequence
ttgtgatgcagacccttccgt

SEQ ID NO: 33 - DNA, 21 bp, Artificial Sequence
  Synthetic sequence
tggaaggcgtcgttctccttt

SEQ ID NO: 34 - DNA, 22 bp, Artificial Sequence
  Synthetic sequence
cctacctcgcgcagaattgttc

(19) United States
(12) Patent Application Publication
(10) Pub. No.: US 2024/0299401 A1
(43) Pub. Date: Sep. 12, 2024

(54) NOVEL REDOX-BASED THERAPEUTIC APPROACH FOR TREATMENT OF CANCER

(71) Applicant: THE PENN STATE RESEARCH FOUNDATION, University Park, PA (US)

(72) Inventor: Sougat MISRA, State College, PA (US)

(21) Appl. No.: 18/030,390

(22) PCT Filed: Oct. 6, 2021
(86) PCT No.: PCT/US2021/053846

§ 371 Date: Apr. 5, 2023

Related U.S. Application Data
(60) Provisional application No. 63/088,157, filed on Oct. 6, 2020.

(51) Int. Cl. A61K 31/5377; A61K 31/095; A61K 31/132; A61K 31/136; A61K 31/166; A61K 31/198; A61K 31/444; A61K 31/4709; A61K 31/555; A61K 33/04; A61K 38/06; A61P 35/00; C12N 9/99; C12N 15/113

(52) U.S. Cl. CPC A61K 31/5377; A61K 31/095; A61K 31/132; A61K 31/136; A61K 31/166; A61K 31/198; A61K 31/444; A61K 31/4709; A61K 31/555; A61K 33/04; A61K 38/063; A61P 35/00; C12N 9/99; C12N 15/113; C12N 2310/14

(57) ABSTRACT

Disclosed herein are compositions and methods for increasing the expression of human SLC7A11. The compositions include a combination of compounds that exhibit anti-proliferative activity against cancer cells. The compositions and methods can be used to treat a subject with cancer. Disclosed are also compositions and methods of treating a coronavirus infection, Zika virus, influenza virus infection, human immunodeficiency virus (HIV), or Rhinovirus.

### CROSS-REFERENCE TO RELATED APPLICATIONS

The application claims the benefit of U.S. Provisional Application No. 63/088,157, filed Oct. 6, 2020, which is hereby incorporated herein by reference in its entirety.

### BACKGROUND

Cell division, energy production, and the maintenance of redox balance are fundamental, intertwined, and indispensable aspects for survival of living cells across the spectrum of taxa in the evolutionary tree. Sustained perturbations of any the above processes have severe consequence on the fitness of living cells with pathophysiological consequences.

Each of these physiological processes, including cellular redox balance, are tunable to therapeutic interventions. Systemic and cellular redox anomalies are signatures of many human diseases and conditions including cancer, inflammatory disorders, fibrosis, neurodegenerative disorders including aging, bacterial and viral infections, radiation injury, ischemic stress, and diabetes among pertinent others.

An implicit need in the era of modern medicine is how to tune different cellular/systemic redox processes to attain therapeutic benefits. Cellular and systemic redox-regulatory systems can be pharmacologically tweaked in an unforeseen manner to confer therapeutic gains, specifically in the context of redox dysregulations implicated in causal or consequential pathophysiological effects. This can be achieved by adopting two different approaches that are completely opposite in nature. The first one is by increasing the antioxidant capacity of cells or at systemic level so that oxidative stress-associated diseases or conditions can be mitigated, managed, or cured depending on the causal or consequential relationship between stressors and the pathophysiological disorders. The opposite approach involves inducing oxidative insults to eliminate a target cell population implicated in a state of disease or condition (e.g., cancer or infection-causing agent).

While both the strategies are therapeutically tractable, their implementation requires identification of versatile small molecules that exert the intended outcomes i.e., either as agent/s that bolster cellular/systemic antioxidant capacity and vice versa, depending on their contextual selective use as antioxidant or pro-oxidant agent/s. An imminent application of pro-oxidative nature of any such compounds is for the treatment of malignancies in which the coveted therapeutic goal is systemic elimination of cancer cells. This is aided by inherent high levels of reactive oxygen species in cancer cells.

The compositions and methods disclosed herein address these and other needs.

### SUMMARY

Provided herein is a pharmaceutical composition including: one or more cytotoxic agent(s), one or more cytotoxicity enhancing agent(s), and one or more pharmaceutically acceptable carriers. In some embodiments, the one or more cytotoxic agent(s) is selected from a compound of category 1, category 2, or a combination thereof. In some embodiments, the one or more cytotoxicity enhancing agent(s) is selected from a compound of category 3, category 4, category 5, a synthetic RNA encoding human SLC7A11 gene, a cystine/glutamate transporter protein (also known as xCT) encoded by human SLC7A11 gene or a combination thereof. In some embodiments, the presence of the cytotoxicity enhancing agent increases the cytotoxicity of the composition by at least 1.5-fold compared to in the absence of the cytotoxicity enhancing agent.

In some embodiments, the cytotoxicity enhancing agent is present in an effective amount to increase the expression of human SLC7A11 gene. In some embodiments, the cytotoxicity enhancing agent is present in an effective amount to increase the expression of human cystine/glutamate transporter protein encoded by human SLC7A11 gene. In some embodiments, the cytotoxicity enhancing agent is present in an effective amount to increase the concentration of human cystine/glutamate transporter protein encoded by human SLC7A11 gene. In some embodiments, the cytotoxicity enhancing agent is present in an effective amount to activate the overexpression of NFE2L2 or ATF4 transcription factors, either individually or simultaneously.

In some embodiments, the ratio of cytotoxic agent to cytotoxicity enhancing agent in the composition ranges from 100:1 to 1:100. In some embodiments, the cytotoxic agent is a compound of category 1. In some embodiments, the cytotoxic agent is a compound of category 2. In some embodiments, the cytotoxic agents are compounds of categories 1 and 2. In some embodiments, the one or more cytotoxicity enhancing agent(s) is a compound of categories 3, 4 or 5 defined by Formula I. In some embodiments, the one or more cytotoxicity enhancing agent(s) is a synthetic RNA encoding human SLC7A11 gene. In some embodiments, the one or more cytotoxicity enhancing agent(s) is a cystine/glutamate transporter protein encoded by human SLC7A11 gene.

Provided herein are also methods for treating cancer comprising administering to a subject in need thereof a therapeutically effective amount of the pharmaceutical composition described herein.

In some embodiments, the subject has a lymphoma, myeloid leukemia, bladder cancer, brain cancer including glioblastoma, head and neck cancer, kidney cancer, lung cancer, myeloma, melanoma, ovarian cancer, cervical cancer, bone cancer, thyroid cancer, adrenal gland cancer, cholangiocarcinoma, pancreatic cancer, prostate cancer, skin cancer, liver cancer, testicular cancer, colon cancer, or breast cancer.

In some embodiments, the pharmaceutical composition is administered by one or more routes selected from the group consisting of buccal, sublingual, intravenous, subcutaneous, intradermal, transdermal, intraperitoneal, oral, eye drops, parenteral, and topical administration.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is a table showing the cytotoxicity of diphenyl diselenide in normal primary human hepatocytes (n=7). Mean values.

FIG. 2 is a graph showing the cytotoxicity of sodium selenite and selenocystine either alone or in combination with diphenyl diselenide in primary human hepatocytes.

FIG. 3 is a graph showing the cytotoxicity of sodium selenite and selenocystine either alone or in combination with diphenyl diselenide in primary human peripheral blood mononuclear cells.

FIG. 4 is a graph showing that diphenyl diselenide dramatically increases selenium uptake from sodium selenite or selenocystine. H1299 cells were treated with different selenium compounds at the mentioned concentrations for 6 hours. Cellular total selenium uptake was measured using ICP-MS. Increased selenium delivery to the cells (from sodium selenite or selenocystine) by diphenyl diselenide warrants increased cytotoxicity in cancer cells, as outlined before.

FIG. 5 is a graph showing that the supply of cystine is important for selenite cytotoxicity in HL60. Cells were cultured either in the absence or presence of cystine (C), methionine (M) and glutamine (G). In addition, only cystine was removed as well. Presented values indicate 24 hours IC₅₀ values for sodium selenite in the specified test condition.

FIG. 6A-6B is a graph showing that combinations of either sodium selenite (6A) or selenocystine (6B) with diphenyl diselenide lead to synergistic cytotoxic effects in HL60 cell line. An 8×8 dose matrix was used for this purpose with incremental doses of sodium selenite/selenocystine in combination with diphenyl diselenide. Synergistic effects were calculated using the package ‘synergyfinder’ in R. Note that increased levels of diphenyl diselenide dramatically strengthen the cytotoxic effects of sodium selenite and selenocystine, while diphenyl diselenide itself exerts limited cytopathic effects.

FIG. 7A-7B is a graph showing that combinations of either sodium selenite (7A) or selenocystine (7B) with diphenyl diselenide lead to synergistic cytotoxic effects in H1299 cell line. An 8×8 dose matrix was used for this purpose with incremental doses of sodium selenite/selenocystine in combination with diphenyl diselenide. Synergistic effects were calculated using the package ‘synergyfinder’ in R. Note that increased levels of diphenyl diselenide dramatically increase the cytotoxic effects of sodium selenite and selenocystine, while diphenyl diselenide itself exerts limited cytopathic effects.

FIG. 8 is a graph showing the toxicity of sodium selenite and selenocystine alone or in combination with diphenyl diselenide at two different doses of diphenyl diselenide (50 and 100 mg/kg body weight) in black mice (C57BL/6 strain).

FIG. 9 is a graph showing the toxicity of sodium selenite (3 mg/kg body weight), selenocystine (3 mg/kg body weight), or in combinations with two different doses of diphenyl diselenide (50 and 100 mg/kg body weight) for 3 weeks in black mice (C57BL/6 strain).

FIG. 10 is a graph showing the toxicity diphenyl diselenide in SCID mice at two different dose levels (1.0 and 3.5 mg/kg body weight).

FIG. 11 is a graph showing the antitumor efficacy of sodium selenite and selenocystine alone or in combination with diphenyl diselenide in SCID mice harboring human acute myeloid leukemia.

FIG. 12 is a graph showing how the difference in tumor volume skewed the results as demonstrated in FIG. 11.

FIG. 13 is a table summarizing the results from immunohistochemistry staining of CK19 (epithelial cell marker) and Ki67 (marker for cell proliferation) following 72 hour treatment with the sodium selenite, selenocystine, and diphenyl diselenide alone or in combinations on the outgrowth and proliferation of cancer cells in human ex vivo organotypic slice culture of surgically resected primary pancreatic ductal adenocarcinoma obtained from a pancreatic cancer patient (This model is described by Misra et al., Scientific Report, 2019). Similar results were obtained from experiments with 4 other cases.

FIG. 14 is a graph of the outgrowth of cells in the periphery of tissue slices (a key parameter of tissue health in ex vivo culture) measured in the different treatment groups. Outgrowth=Percentage of the tissue periphery covered by the migrating cells (a characteristic similar to wound healing process).

FIG. 15A-15B is a graph of SLC7A11 mRNA expression following treatment with the indicated compounds. HL 60 (15A) and H1299 (15B) cells were treated with diphenyl diselenide (5 μM), Tert-butyl hydroxyquinone (5 μM), and Tunicamycin (2.5 μM) for 6 h.

FIG. 16A-16B are graphs showing that SLC7A11 expression is important for selenite and selenocystine cytotoxicity. FIG. 16A. H1299 cells were transfected with either mock or SLC7A11 overexpression plasmid. 24 hours post transfection, cells were harvested and replated in 96-well plate. Following overnight incubation, cells were treated with selenite or selenocystine and cytotoxicity was measured using WST-1 assay. FIG. 16B. To demonstrate the opposite effects, H1299 cells were transfected with either scrambled or two SLC7A11 siRNAs individually. Values indicate 24 h IC₅₀ values.

FIG. 17 is a graph of NFE2L2-inducing activity of the selected compounds. MCF7 cells expressing stable NFE2L2 (NFR2) inducible luciferase construct were treated with the selected compounds for 24 h. Tert-butyl hydroxyquinone (50 μM) served as a positive control.

FIG. 18 is a graph of ATF4-inducing activity of the selected compounds. HEK293 cells expressing stable ATF4 inducible luciferase construct were treated with the selected compounds for 24 h. Bortezomib (50 nM) served as a positive control.

FIG. 19 is a graph showing sodium selenite increases the cytotoxicity of sorafenib, an anticancer drug. HepG2 cells were treated with either sodium selenite or sodium selenite in combination with sorafenib (5 μM) for 24 h and cytotoxicity was measured using WST-1 assay.

FIG. 20 is a table summarizing the results from immunohistochemistry staining of pS6 (marker for metabolic activity) in these slices following 72 h treatments with the respective compounds either alone or in specified combinations.

FIG. 21 is an illustration of in-silico docking analysis showing occupancy and potential interaction with key cysteine residues (including Cys145 of the active site) of SARS-CoV-2 M protein with the model compound phenylselenol (dots). This interaction can be bolstered in the presence of sodium selenite, selenium dioxide, sodium selenide, selenious acid, or a combination thereof.

FIG. 22 is a proposed mechanism of action of the compounds of Formula II in different combinations, for the inhibition of the main viral protease.

FIG. 23 is a graph showing viability of mice splenocytes (leukemic cell count >70%) following exposure with the indicated compounds (n=4).

FIG. 24 is a graph showing viability of splenocytes ex vivo isolated from secondary leukemic mice following exposure with the indicated compounds.

FIG. 25 is a graph showing splenocytes from secondary leukemic mice are isolated and cultured ex vivo in 24 well plates for 12 h prior to the treatment with the indicated compounds either alone or in combinations for another 48 h.

FIG. 26 is a graph showing apoptotic cell death in isolated bone-marrow resident human leukemic cells ex vivo following the combined treatment of DPDS and sodium selenite.

FIG. 27 is a schematic depicting the proposed mechanisms of action of the combinatorial therapies, as outlined in this proposal. Note that the proposed strategy relies on activation of SLC7A11 by DPDS, a non-toxic compound. When DPDS is combined with sodium selenite or selenocystine, malignant cells undergo unprecedented redox stress due to preexisting amplification of oxidative processes and die. See details in the text.

FIG. 28A-28J (28A) Cytotoxicity of SS and SeCySS alone or in combination with DPDS in 5 different leukemia cell lines. (28B) Cytotoxicity of SS and SeCySS alone or in combination with DPDS in primary human PBMCs. Normal PBMCs are resistant to the combined treatments ex vivo. (28C and 28D) Drug interaction analyses following combined treatments of DPDS with either selenite or selenocystine for 24 h in HL60 cells. A ZIP score of 0 implies both probabilistic independence and dose additivity, whereas a ZIP score of <0 indicates antagonistic drug effects and a score of >0 indicates synergistic drug effects. (28E) Relative tumor volume in tumor-bearing (human HL60 cell xenotransplant) NSG mice following treatment (3 days a week) with the mentioned compounds for 17 days. (28F) Body and organ weight following treatment (3 days a week) with the compounds at indicated dose levels. Toxicity of sodium selenite following (28G) SLC7A11 overexpression, (28H) siRNA-based knock-down, and (28I) pharmacological inhibition of cystine transport in H1299 cells (used for ease of transfection). (28J) DPDS increases intracellular GSH levels and extracellular CySH levels in HL60 cells. However, the combined treatments significantly reduce intracellular GSH to GSSG ratio, suggesting involvement of redox stress. SS—Sodium selenite, SeCySS—Selenocystine, DPDS—Diphenyl diselenide, MSG—monosodium glutamate GSH—Glutathione, GSSG—Oxidized glutathione, CySH—Cysteine, CYSS—Cystine.

FIG. 29 of a scheme of the generation of AML in mice and selected end-points in phase-I and phase-II studies.

FIG. 30 is treatment schedule in secondary leukemic mice in phase-I and phase-II studies. In this model, WBC count exceeds >10000/microliter (reminiscent of human leukemia) after 10 days when treatment is initiated.

FIG. 31A-31H shows SLC7A11 regulates extracellular redox potential. 31A. Kaplan Meier survival analysis demonstrating higher SLC7A11 mRNA expression is associated with poor outcome in cancer patients. Data source: UCSC Xena genome browser comprising TCGA and TARGET Pan-Cancer (PANCAN) database covering all cancer types. 31B. Comparative mRNA expression of SLC7A11 between normal (GTEx, Genotype-tissue Expression database, all tissue types) and cancerous tissues (TARGET and TCGA database, pan-cancer). Kruskal-Wallis test followed by multiple comparison with two-stage step-up method of Benjamini, Krieger and Yekutieli to correct for false discovery rate (KW statistic=2482, P<0.0001). IQR—Inter quartile range. 31C. Extracellular thiols levels (24 h) in 23 different cancer cells lines of multiple origins (n=3-17). 31D. Correlation between SLC7A11 expression and extracellular reduced thiols in the selected cell lines. Basal levels of SLC7A11 expression data in these cell lines were extracted from Cancer Cell Line Encyclopedia (CCLE) database. 31E. The levels of extracellular total thiols in 4 different cell lines following plasmid-based ectopic overexpression of SLC7A11 in 4 different cell lines (n=6-11, Student's t-test). 31F. Erastin-induced (5 μM) inhibits in the extracellular reduced thiols levels in all the tested cell lines (n=4-12, Student's t-test). 31G. The levels of extracellular total thiols following transient knock-down of SLC7A11 in 4 different cell lines (n=5-10, one-way ANONA followed by Holm-Sidak method of multiple comparison). 31H. A549 cells stably transfected with either eGFP or two SLC7A11 shRNA clones were cultured in high glucose and cystine-containing RPMI or low glucose and cystine-containing F12 (ATCC recommended culture media for A549 cells). A549 cells transfected with shRNA clone 926 exhibited lower levels of extracellular reduced thiols compared to control or eGFP-transfected A549 cells (n=5-8, one-way ANONA followed by Holm-Sidak method of multiple comparison).

FIG. 32A-32F shows regulation of extracellular redox potential by NFE2L2. 32A. Time-course changes in the total extracellular thiols in HL60 and H1299 cells following treatment with the compounds at the indicated dose levels (n=3, one-way ANOVA followed by multiple comparison with Dunnett's test). 32B. mRNA expression of NFE2L2 and its target genes in HL60 cell line following treatment with DPDS (5.0 μM), tert-butyl hydroxyquinone (t-BHQ, 5.0 μM), and tunicamycin (TU, 2.5 μM) for 24 h (n=4, one-way ANONA followed by Holm-Sidak method of multiple comparison). 32C. mRNA expression of NFE2L2 and its target genes in H1299 cell line following treatment with the selected compounds for 24 h. (n=4, one-way ANONA followed by Holm-Sidak method of multiple comparison). 32D. NFE2L2 activity (left panel) and corresponding extracellular total thiols levels (right panel) in NFE2L2 reporter MCF7 cell line following treatment with the selected small molecules. Except for All-trans retinoic acid (1 μM) and Tunicamycin (2.5 μM), the concentration of all the tested compounds were 5 μM unless noted otherwise. Tert-butyl hydroxyquinone (50 μM) served as the positive control (n=4-13, one-way ANOVA followed by multiple comparison with Dunnett's test). 32E. Kinetics of NFE2L2 activity and extracellular total thiols in NFE2L2 reporter MCF7 cells following treatment with increasing doses of DPDS (n=4) at 6 and 24 h. 32F. Correlation between NFE2L2 activity and extracellular total thiols following treatment with the indicated compounds at fixed dose levels of (5 μM).

FIG. 33A-33G shows ATF4-mediated regulation of SLC7A11 expression controls extracellular redox potential. 33A. ATF4 activity (left panel) and corresponding extracellular total thiols levels (right panel) in NFE2L2 reporter MCF7 cell line following treatment with the selected small molecules. The concentrations of these compounds were same as in FIG. 32D. Bortezomib (50 nM) served as the positive control (n=4-5, one-way ANOVA followed by multiple comparison with Dunnett's test). 33B. mRNA expression of ATF4 pathway genes in HL60 and H1299 cell line following treatment with the selected compounds for 24 h. (n=4, one-way ANONA followed by Holm-Sidak method of multiple comparison). 33C. Kinetics of ATF4 activity and extracellular total thiols in NFE2L2 reporter HEK293 cells following treatment with increasing doses of DPDS (n=4) at 6 and 24 h. 33D. Extracellular reduced thiols levels in wild-type (WT) or ATF4 knock-out (KO) HeLa cells at 24 h (n=9, Student's t-test). 33E. Expression of SLC7A11, SLC3A2, and CD44 mRNA in HL60 and H1299 cells following treatment with the test compounds for 24 h as outlined above (n=4, one-way ANONA followed by Holm-Sidak method of multiple comparison). 33F. Human xCT gene promoter analysis using a luciferase-based assay. Details of the plasmids can be found in Ye. et al., 2014. H1299 cells were transfected with either a wild-type reporter gene (pxCTpro WT-Luc) or mutated reporter constructs and then treated with the test compounds for 24 h. The heatmap shows relative reporter activity (n=4). 33G. Correlation between NFE2L2 activity and extracellular total thiols following treatment with the indicated compounds at fixed dose levels of (5 μM).

FIG. 34A-34G shows cysteine efflux is regulated by the availability of cystine, methionine, and glutamine. 34A. A schematic describing metabolic transformation of cystine and methionine into cysteine. Cystine is transported via SLC7A11 and reduced intracellularly to form cysteine. Conversion of glutamine to glutamate supports the activity of this transporter by supplying its co-transport molecule. On the other side, methionine is converted into cysteine via transsulfuration pathway. Excess cysteine is effluxed out of the cell to maintain a homeostatic intracellular redox buffer. 34B. Basal levels of extracellular reduced thiols in HL60 cells 6 h after seeding, as measured by HPLC (n=4). ND—not detectable. 34C. HepG2 cells were serum-starved for 48 h for cell cycle synchronization and replenished with complete culture medium for another 24 h. Thereafter, cells were cultured either in the presence or absence of cystine, methionine, glutamine, and DPDS and extracellular reduced thiols levels were measured at the indicated time points (n=4, one-way ANONA followed by Holm-Sidak method of multiple comparison). 34D. Extracellular reduced thiols levels in MCF7 cells in a similar experimental set up as in FIG. 4C (n=4-8, one-way ANONA followed by Holm-Sidak method of multiple comparison). 34E. Extracellular reduced thiols levels in MCF7 (top panel) and HepG2 cells (bottom panel) cultured in complete medium following treatment with DPDS (5 μM), Erastin (5 μM), and BCH for 24 h (n=3-8, one-way ANONA followed by Holm-Sidak method of multiple comparison). 34F. Kinetics of extracellular reduced thiols in MCF7 cells following short-term treatment with BSO, DPDS or both. 34G. Extracellular reduced thiols levels in A549 and H1299 cells cultured in complete medium following treatment with Erastin (5 μM), mono-sodium glutamate (MSG, 50 mM), Sorafenib (2.5 μM), and trans-2,4-PDC (100 μM) for 24 h (n=3, one-way ANONA followed by Holm-Sidak method of multiple comparison).

FIG. 35A-35F shows SLC7A11 expression and acquisition of cystine and methionine play key roles in sodium selenite and selenocystine cytotoxicity. 35A. Plasmid-based overexpression of SLC7A11 sensitizes cancer cells to sodium selenite and selenocystine (n=4-9, Student's t-test). Highly drug resistant U1906L cells were used to test the efficacy of the test compounds in exerting cytotoxicity. 35B. siRNA-mediated transient knock-down of SLC7A11 abrogates the cytotoxicity of sodium selenite and selenocystine (n=3-6, one-way ANONA followed by Holm-Sidak method of multiple comparison). 35C. Cytotoxicity of these selenium compounds in A549 cells (wild-type, eGFP transfected, or SLC7A11 knock-out clones) cultured either in high (RPMI medium) or low (F12 medium) cystine containing medium (n=3-5, one-way ANONA followed by Holm-Sidak method of multiple comparison). 35D. Top panel, viability of H1299 cells when cultured in medium either in the absence or presence of cystine, methionine, and glutamine (n=6). Bottom panels, cytotoxicity of the test compounds in H1299 cells when cultured in these media for 24 h (n=4, one-way ANONA followed by Holm-Sidak method of multiple comparison). 35E. Cytotoxicity of the test compounds in HepG2 cells when cultured in these media for 24 h (n=4-6, one-way ANONA followed by Holm-Sidak method of multiple comparison).

FIG. 36A-36G shows small molecule-induced modulation of extracellular thiols exerts selective cytotoxicity in cancer cells. 36A. Cytotoxicity of sodium selenite and selenocystine upon co-treatment with DPDS (5 μM) in 23 different cancer cell lines of multiple cancer origins (n=3-17). In all the cases, cytotoxicity was higher in the combinatorial treatments compared to single treatments alone. Shaded pink area shows reference plasma total selenium concentration (0.88-1.65 μM) in healthy human subjects. 36B. Synergy analysis for the combinatorial treatments in H1299 cells (n=4). A higher ZIP score indicates higher cytotoxicity and vice versa. 36C. Cytotoxicity of these selenium compounds in primary human hepatocytes (n=7-8) and in primary human PBMCs (n=3-6).

FIG. 37 is a schematic depicting a simplified mechanism of actions, as outlined in this proposal. DPDS induces SLC7A11 expression and facilitates cellular uptake of selenocystine. High cystine influx by SLC7A11 also increases cysteine levels in the extracellular milieu which augment the cellular uptake of sodium selenite. These selenium-containing small molecules redox-cycle with GSH and cysteine, resulting in ROS generation and loss of GSH homeostasis. Resulting irreversible loss of redox balance leads to cell death.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention.

### Definitions

To facilitate understanding of the disclosure set forth herein, a number of terms are defined below. Unless defined otherwise, all technical and scientific terms used herein have the same meanings as commonly understood by one of skill in the art to which the disclosed invention belongs. Publications cited herein and the materials for which they are cited are specifically incorporated by reference.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compositions, articles, and methods, examples of which are illustrated in the accompanying Examples.

### Pharmaceutical Compositions

Described herein is a pharmaceutical composition including: one or more cytotoxic agent(s), one or more cytotoxicity enhancing agent(s), and one or more pharmaceutically acceptable carriers. In some embodiments, the one or more cytotoxic agent(s) is selected from a compound of category 1, category 2, category 3 or a combination thereof. In some embodiments, the one or more cytotoxic agent(s) is selected from a compound of category 1, category 2, or a combination thereof. In some embodiments, the one or more cytotoxic enhancing agent(s) is selected from a compound of category 3, category 4, category 5, a synthetic RNA encoding human SLC7A11 gene, a cystine/glutamate transporter protein encoded by human SLC7A11 gene, or a combination thereof.

In some embodiments, the presence of the cytotoxicity enhancing agent increases the cytotoxicity of the composition by at least 1.5-fold compared to in the absence of the cytotoxicity enhancing agent. For example, the cytotoxicity of the composition increases by at least 2-fold, at least 4-fold, at least 6-fold, at least 8-fold, at least 10-fold, at least 20-fold, at least 40-fold, at least 60-fold, at least 80-fold, at least 100-fold, at least 150-fold, at least 200-fold, at least 250-fold, at least 300-fold, at least 350-fold, at least 400-fold, at least 600, or at least 800. For example, the presence of the cytotoxicity enhancing agent increases the cytotoxicity of the composition from 1.5-fold to 800-fold (e.g., from 1.5-fold to 3-fold, 1.5-fold to 5-fold, 1.5-fold to 10-fold, 1.5-fold to 50-fold, 1.5-fold to 100-fold, 1.5-fold to 150-fold, 1.5-fold to 200-fold, 1.5-fold to 250-fold, 1.5-fold to 300-fold, 1.5-fold to 350-fold, 1.5-fold to 400-fold, 1.5-fold to 450-fold, 1.5-fold to 500-fold, 1.5-fold to 550-fold, 1.5-fold to 600-fold, 1.5-fold to 650-fold, 1.5-fold to 700-fold, 1.5-fold to 750-fold, 5-fold to 10-fold, 5-fold to 50-fold, 5-fold to 100-fold, 5-fold to 150-fold, 5-fold to 200-fold, 5-fold to 250-fold, 5-fold to 300-fold, 5-fold to 350-fold, 5-fold to 400-fold, 5-fold to 450-fold, 5-fold to 500-fold, 5-fold to 550-fold, 5-fold to 600-fold, 5-fold to 650-fold, 5-fold to 700-fold, 5-fold to 750-fold, 10-fold to 50-fold, 10-fold to 100-fold, 10-fold to 150-fold, 10-fold to 200-fold, 10-fold to 250-fold, 10-fold to 300-fold, 10-fold to 350-fold, 10-fold to 400-fold, 10-fold to 450-fold, 10-fold to 500-fold, 10-fold to 550-fold, 10-fold to 600-fold, 10-fold to 650-fold, 10-fold to 700-fold, 10-fold to 750-fold, 50-fold to 100-fold, 50-fold to 150-fold, 50-fold to 200-fold, 50-fold to 250-fold, 50-fold to 300-fold, 50-fold to 350-fold, 50-fold to 400-fold, 50-fold to 450-fold, 50-fold to 500-fold, 50-fold to 550-fold, 50-fold to 600-fold, 50-fold to 650-fold, 50-fold to 700-fold, 50-fold to 750-fold, from 100-fold to 150-fold, 100-fold to 200-fold, 100-fold to 250-fold, 100-fold to 300-fold, 100-fold to 350-fold, 100-fold to 400-fold, 100-fold to 450-fold, 100-fold to 500-fold, 100-fold to 550-fold, 100-fold to 600-fold, 100-fold to 650-fold, 100-fold to 700-fold, 100-fold to 750-fold, 250-fold to 300-fold, 250-fold to 350-fold, 250-fold to 400-fold, 250-fold to 450-fold, 250-fold to 500-fold, 250-fold to 550-fold, 250-fold to 600-fold, 250-fold to 650-fold, 250-fold to 700-fold, 250-fold to 750-fold, 350-fold to 400-fold, 350-fold to 450-fold, 350-fold to 500-fold, 350-fold to 550-fold, 350-fold to 600-fold, 350-fold to 650-fold, 350-fold to 700-fold, or 350-fold to 750-fold. In some embodiments, the cytotoxicity enhancing agent is present in an effective amount to increase the expression of human SLC7A11 gene; increase the concentration of the human cystine/glutamate transporter protein encoded by human SLC7A11 gene; activate the overexpression of NFE2L2, ATF4, or both transcription factors; or any combination thereof. In some embodiments, the cytotoxicity enhancing agent is present in an effective amount to increase the expression of human SLC7A11 gene. In some embodiments, the cytotoxicity enhancing agent is present in an effective amount to increase the concentration of the human cystine/glutamate transporter protein encoded by human SLC7A11 gene. In some embodiments, the cytotoxicity enhancing agent is present in an effective amount to activate the overexpression of NFE2L2, ATF4, or both transcription factors.

In some embodiments, the cytotoxic agent and the cytotoxicity enhancing agent in the composition are present in a ratio of cytotoxic agent to cytotoxicity enhancing agent of least 1:100 (e.g., 1:50, 1:40, 1:20, 1:10, 1:5, 1:4, 1:2, 1:1, 2:1, 5:1, 5:2, 10:1, 25:1, 20:1, 40:1, 50:1, or 80:1). In some embodiments, the cytotoxic agent and the cytotoxicity enhancing agent in the composition are present in a ratio of cytotoxic agent to cytotoxicity enhancing agent of 100:1 or less (e.g., 80:1 or less, 50:1 or less, 40:1 or less, 20:1 or less, 25:1 or less, 10:1 or less, 5:2 or less, 5:1 or less, 2:1 or less, 1:1 or less, 1:2 or less, 1:4 or less, 1:5 or less, 1:10 or less, 1:20 or less, 1:40 or less, or 1:50 or less). The cytotoxic agent and the cytotoxicity enhancing agent in the composition are present in a ratio of cytotoxic agent to cytotoxicity enhancing agent of ranging from any of the minimum values described above to any of the maximum values described above. For example, in some embodiments, the cytotoxic agent and the cytotoxicity enhancing agent in the composition are present in a ratio ranging from 1:100 to 100:1 (e.g., 1:100 to 80:1, 1:100 to 60:1, 1:100 to 40:1, 1:100 to 20:1, 1:100 to 10:1, 1:100 to 5:1, 1:100 to 2:1, 1:100 to 1:1, 1:100 to 1:2, 1:100 to 1:4, 1:100 to 1:5, 1:100 to 1:10, 1:100 to 1:20, 1:100 to 1:40, 1:100 to 1:50, or 1:100 to 1:80). In some embodiments, the cytotoxic agent and the cytotoxicity enhancing agent in the composition are present in a ratio of cytotoxic agent to cytotoxicity enhancing agent of 1:100, 1:50, 1:40, 1:20, 1:10, 1:5, 1:4, 1:2, 1:1, 2:1, 5:1, 5:2, 10:1, 25:1, 20:1, 40:1, 50:1, or 80:1, 100:1. In some embodiments, the ratio of cytotoxic agent to cytotoxicity enhancing agent in the composition is 1:1.

In some embodiments, the cytotoxic agent is a compound of category 1. In some embodiments, the cytotoxic agent is a compound of category 2. In some embodiments, the cytotoxic agents are compounds of categories 1 and 2 and can be used in combinations.

In some embodiments, the compound of category 1 is sodium selenite, potassium selenite, selenium dioxide, sodium selenide, potassium selenide, selenious acid, elemental selenium, selenite anion, any precursor molecules that give rise to selenite anion or hydrogen selenide, or a combination thereof.

In some embodiments, the compound of category 2 is:

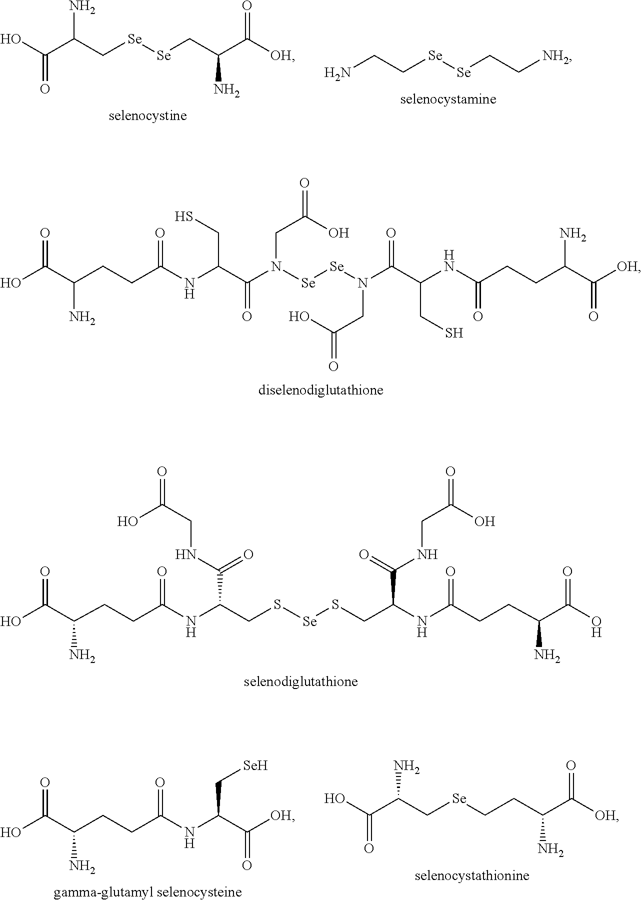

a pharmaceutically acceptable salt thereof, or a combination thereof.

In some embodiments, category 3 compounds can be

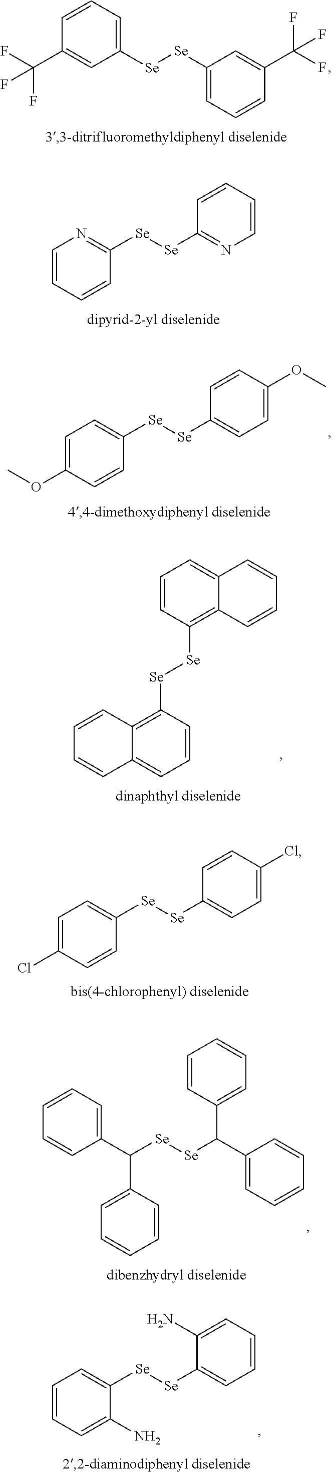

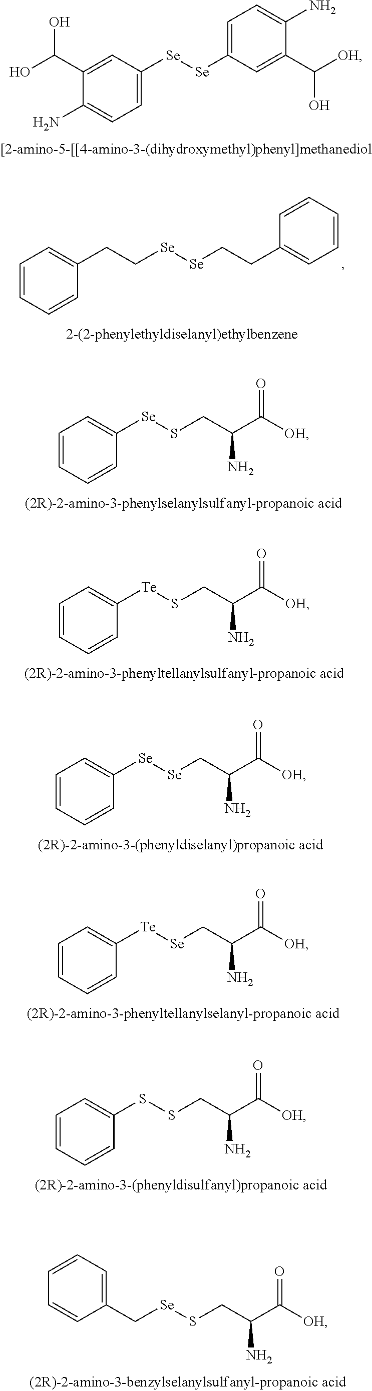

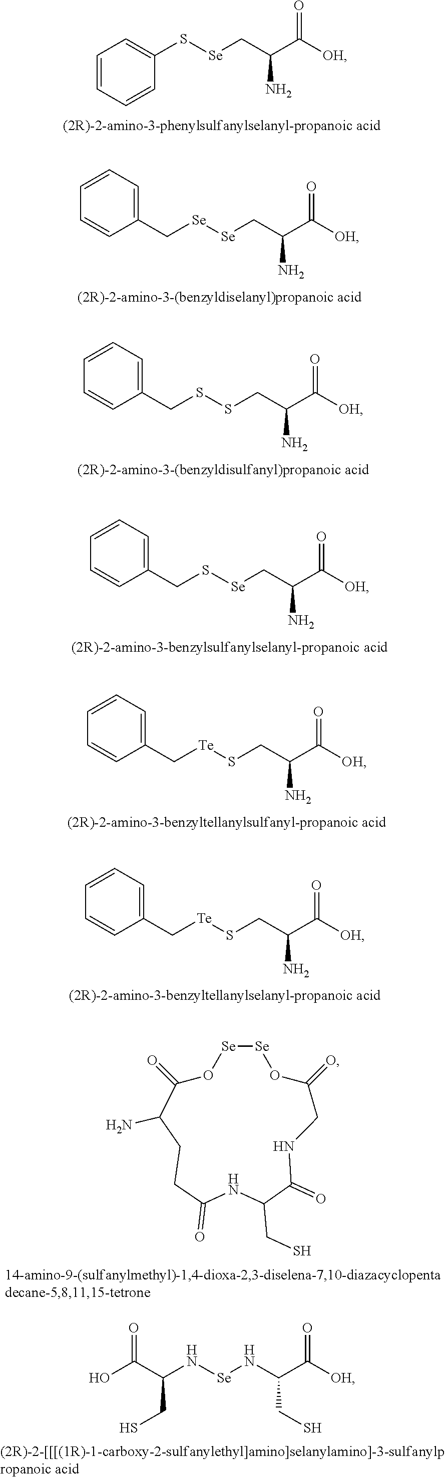

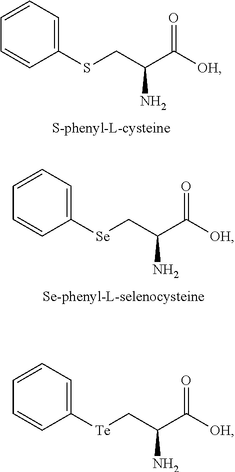

a pharmaceutically acceptable salt thereof, or a combination thereof.

In some embodiments, the category 3 compounds can be a hybrid molecule that serves as both a cytotoxic agent as well as a cytotoxicity enhancing agent.

In some embodiments, category 4 compounds can be methionine, S-adenosyl-L-methionine, selenomethionine, ethacrynate, salubrinal, proteasome inhibitors, bromsulphalein and its selenium analogues in which its sulphur moieties are replaced with selenium molecules, Oltipraz, sulphoraphane, curcumin and its precursors and derivatives, dimethylfumarate, monomethyl fumarate, tert-butyl hydroxyquinone, diethyl maleate, small peptides that inhibits KEAP1 protein interaction with NFE2L2, diphenyl ditelluride, diphenyl disulfide, diphenyl diselenide, dibenzyl ditelluride, dibenzyl disulfide, dibenzyl diselenide or any disulfide (R—S—S—R), diselenide (R—Se—Se—R) or ditellulride (R—Te—Te—R) molecules in which R refers to either symmetric or asymmetric aliphatic or aromatic side chains. One common characteristics of this class of molecules, with certain exceptions, is that these molecules alter the redox potential of the microenvironment of cancer or normal cells or blood at systemic level by effluxing endogenous thiols (mainly cysteine, but also gamma-glutamyl cysteine, glutathione, cystathionine) in human normal or cancer cells. Some of these molecules also increase cellular cystine, cysteine and glutathione contents. Similar properties are exhibited by some of the category 3 molecules as well, since these contain one of the building blocks or functional groups that is implicated in the observed effects of category 4 molecules on modulating the redox potential of the microenvironment of cancer or normal cells.

In some embodiments, category 5 compounds can be (2-(2-(2-((dimethylamino)methyl)phenyl)diselanyl)phenyl)-N,N-dimethylmethanamine, 1,2-bis(2,4-dinitrophenyl)diselane, 1,2-bis(2-morpholinoethyl)diselane, 1,2-bis(isoindoline-1,3-dione-2 ethyl)diselane, 2-(2-(2-((dimethylamino)methyl)-6-methoxyphenyl)diselanyl)-3-methoxyphenyl)-N,Ndimethylmethanamine, 2,2′-di(phenylethyl) diselenide, 2,3-bis(phenylselanyl)naphthalene-1,4-dione, 2′2-diaminodiphenyl diselenide, 3′,5′,3,5-tetratrifluoromethyl-diphenyl diselenide, 3′3-ditrifluoromethyldiphenyl diselenide, 4′,4-diaminodiphenyl diselenide, 4′,4-dimethoxydiphenyl diselenide, 5,8-dihydroxy-2,3-bis(phenyl-selanyl)naphthalene-1,4-dione, 8-(2-(8-(dimethylamino)naphthalen-1-yl)diselanyl)-N,N-dimethylnaphthalen-1-amine, binaphthyl diselenide, bis(4-clorophenyl) diselenide, dibenzhydryl diselenide, dibutyl diselenide, diethyl diselenide, dipropyl diselenide, dipyrid-2-yl diselenide, diquinolyl-8-yl diselenide, p-methoxybenzeneselenol. Category 5 molecules also include sulfur and tellurium analogs in which one or more selenium molecule(s) in these compounds are replaced by sulfur or tellurium atoms as similar redox-modulatory activities are shown for category 4 molecules with certain exceptions. In view of the observed effects of category 4 molecules, the following molecules are expected to exhibit similar biological activities that is these molecules can also increase in the intracellular and extracellular thiols when treated with the following compounds based on their structure-activity relationships.

In some preferred embodiments, the pharmaceutical composition includes a category 1 and category 4 compound. In some preferred embodiments, the pharmaceutical composition includes a category 2 and category 4 compound. In some preferred embodiments, the pharmaceutical composition includes a category 1, category 2, and category 4 compound. In some preferred embodiments, the pharmaceutical composition includes a category 1 and/or category 2 compound, and categories 3, 4, or 5 compounds. For example, the pharmaceutical composition includes a combination of diphenyl diselenide, sodium selenite and/or selenocystine.

In some embodiments, wherein the one or more cytotoxicity enhancing agent(s) of categories 3, 4 or 5 is a compound defined by Formula I:

R₃—X—Y—R₂ Formula I
or a pharmaceutically acceptable salt thereof,
wherein
X is S, Se, or Te;
Y is absent, or is S, Se, or Te;
R₂ and R₃ are independently substituted or unsubstituted alkyl, aryl, heteroaryl, heteroalkyl, or a combination thereof,
wherein when present the alkyl is optionally substituted with an alkyl, aryl, primary amine, secondary amine, tertiary amine, primary amide, secondary amide, tertiary amide, carboxylic acid, thiol, carbonyl, hydroxy, ether, ester, halogen or a combination thereof;
wherein when present the aryl is optionally substituted alkyl, aryl, primary amine, secondary amine, tertiary amine, primary amide, secondary amide, tertiary amide, carboxylic acid, thiol, carbonyl, hydroxy, ether, ester, halogen, —CF₃, or a combination thereof.

In some embodiments, the compound of Formula I is:

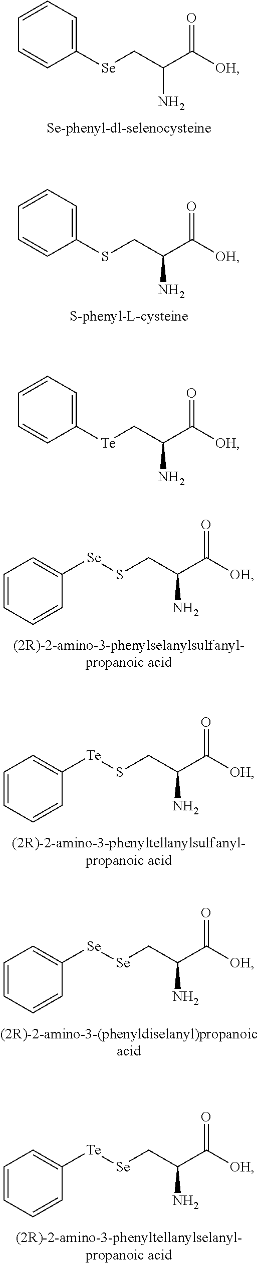

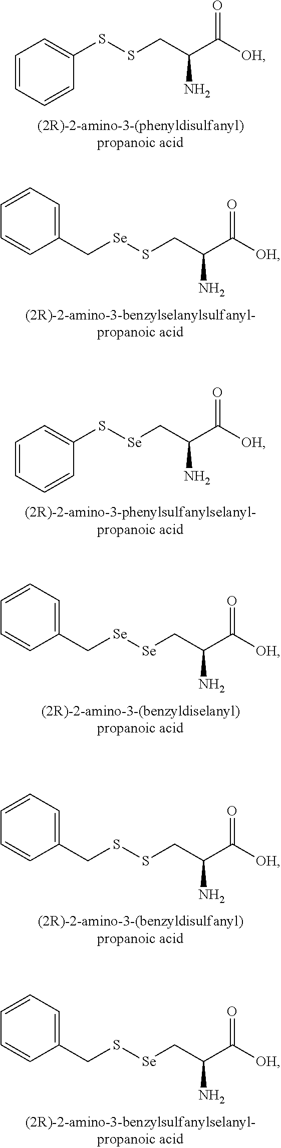 [SMILES: N[C@@H](CSSc1ccccc1)C(=O)O ; N[C@@H](CS[Se]Cc1ccccc1)C(=O)O ; N[C@@H](C[Se]Sc1ccccc1)C(=O)O ; N[C@@H](C[Se][Se]Cc1ccccc1)C(=O)O ; N[C@@H](CSSCc1ccccc1)C(=O)O ; N[C@@H](C[Se]SCc1ccccc1)C(=O)O]

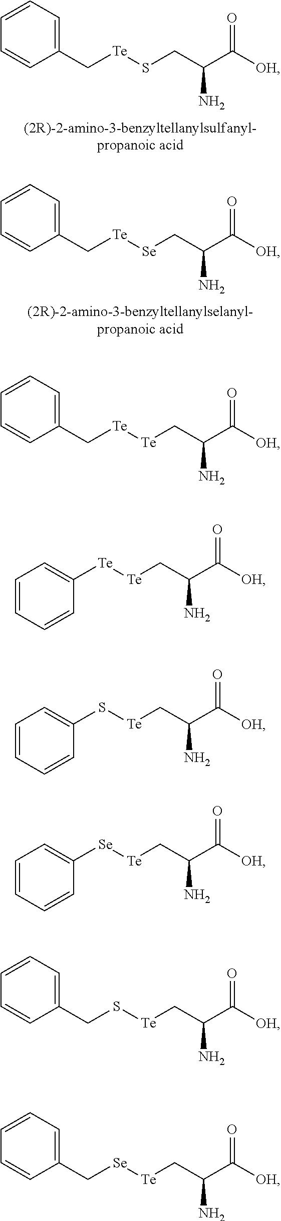

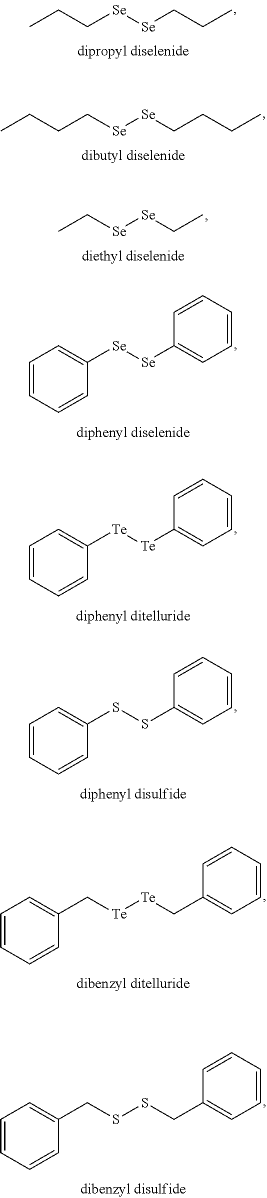

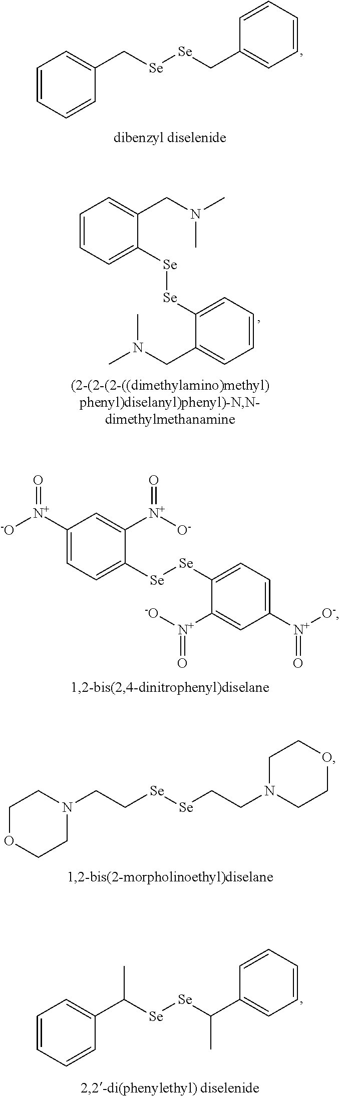

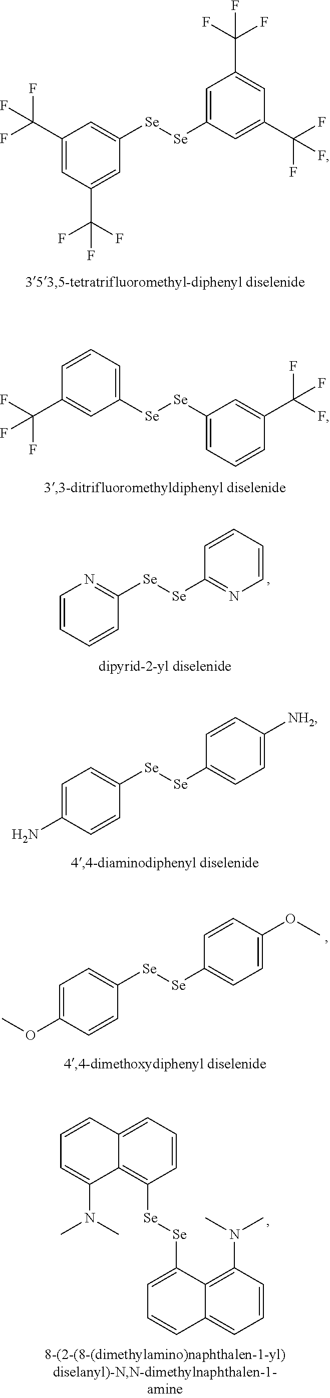

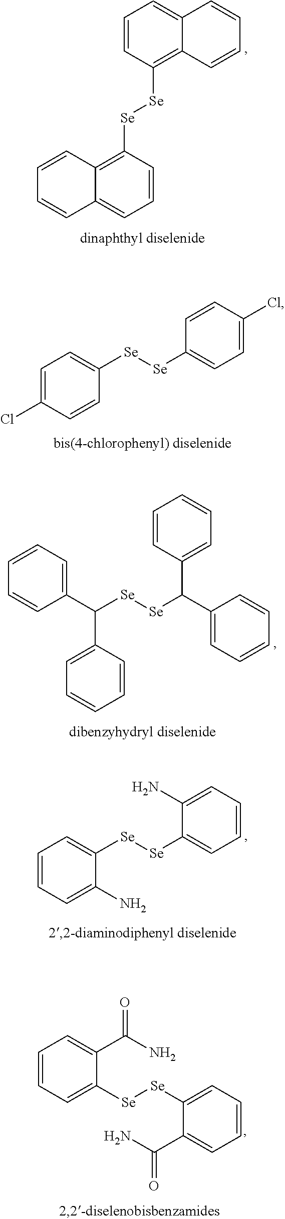 [SMILES: c1ccc2c([Se][Se]c3cccc4ccccc34)cccc2c1 ; Clc1ccc([Se][Se]c2ccc(Cl)cc2)cc1 ; c1ccc(C([Se][Se]C(c2ccccc2)c2ccccc2)c2ccccc2)cc1 ; Nc1ccccc1[Se][Se]c1ccccc1N ; NC(=O)c1ccccc1[Se][Se]c1ccccc1C(N)=O]

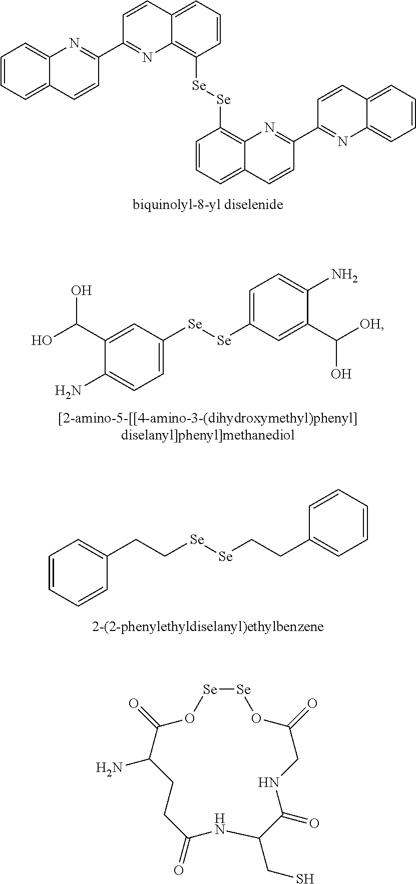 [SMILES: c1ccc2nc(-c3ccc4cccc([Se][Se]c5cccc6ccc(-c7ccc8ccccc8n7)nc56)c4n3)ccc2c1 ; Nc1ccc([Se][Se]c2ccc(N)c(C(O)O)c2)cc1C(O)O ; c1ccc(CC[Se][Se]CCc2ccccc2)cc1 ; NC1CCC(=O)NC(CS)C(=O)NCC(=O)O[Se][Se]OC1=O]

14-amino-9-(sulfanylmethyl)-1,2-dioxa-2,3-diselena-7,10-diazacylopentadecane-5,8,11,15-tetrone, a pharmaceutically acceptable salt thereof, or a combination thereof.
In some embodiments, the compound of Formula I is:

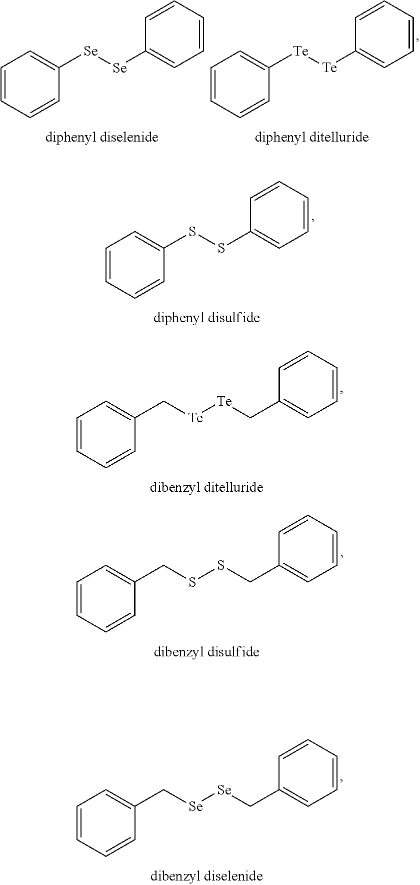

a pharmaceutically acceptable salt thereof, or a combination thereof.

In some embodiments, the one or more cytotoxicity enhancing agent(s) is a synthetic RNA encoding human SLC7A11 gene. In some embodiments, the one or more cytotoxicity enhancing agent(s) is a cystine/glutamate transporter protein encoded by human SLC7A11 gene.

Also described herein are pharmaceutical composition for the treatment of coronaviruses comprising:
one or more compounds having Formula II

R₁—X—R₂, Formula II
or a pharmaceutically acceptable salt thereof,
wherein
R₁ is hydrogen, substitute or unsubstituted alkyl or aryl, or —Y—R₃;
R₂ is substituted or unsubstituted alkyl, aryl, heteroaryl, heteroalkyl, —Y—R₃, or a combination thereof;
X is a S, Se, or Te;
Y is a S, Se, or Te;
R₃ is substituted or unsubstituted alkyl, aryl, heteroaryl, heteroalkyl, or a combination thereof,
wherein when present the alkyl is optionally substituted with an alkyl, aryl, primary amine, secondary amine, tertiary amine, primary amide, secondary amide, tertiary amide, carboxylic acid, thiol, carbonyl, hydroxy, ether, ester, halogen or a combination thereof;
wherein when present the aryl is optionally substituted alkyl, aryl, primary amine, secondary amine, tertiary amine, primary amide, secondary amide, tertiary amide, carboxylic acid, thiol, carbonyl, hydroxy, ether, ester, halogen, —CF₃ or a combination thereof;
a compound selected from sodium selenite, selenium dioxide, sodium selenide, selenious acid or a combination thereof; and
a pharmaceutically acceptable carrier.

In some embodiments, R₁ is —Y—R₃.

In some embodiments, the one or more compounds of Formula II can be defined by Formula I:

R₃—X—Y—R₂ Formula I
or a pharmaceutically acceptable salt thereof,
wherein
X is a S, Se, or Te;
Y is a S, Se, or Te;
R₂ and R₃ are independently substituted or unsubstituted alkyl, aryl, heteroaryl, heteroalkyl, or a combination thereof,
wherein when present the alkyl is optionally substituted with an alkyl, aryl, primary amine, secondary amine, tertiary amine, primary amide, secondary amide, tertiary amide, carboxylic acid, thiol, carbonyl, hydroxy, ether, ester, halogen or a combination thereof;
wherein when present the aryl is optionally substituted alkyl, aryl, primary amine, secondary amine, tertiary amine, primary amide, secondary amide, tertiary amide, carboxylic acid, thiol, carbonyl, hydroxy, ether, ester, halogen, —CF₃ or a combination thereof.

In some embodiments, the pharmaceutical composition for the treatment of coronaviruses comprising:
one or more compounds having Formula I:

R₃—X—Y—R₂ Formula I
or a pharmaceutically acceptable salt thereof,
   wherein
X is a S, Se, or Te;
Y is a S, Se, or Te;
R₂ and R₃ are independently substituted or unsubstituted alkyl, aryl, heteroaryl, heteroalkyl, or a combination thereof,
wherein when present the alkyl is optionally substituted with an alkyl, aryl, primary amine, secondary amine, tertiary amine, primary amide, secondary amide, tertiary amide, carboxylic acid, thiol, carbonyl, hydroxy, ether, ester, halogen or a combination thereof;
wherein when present the aryl is optionally substituted alkyl, aryl, primary amine, secondary amine, tertiary amine, primary amide, secondary amide, tertiary amide, carboxylic acid, thiol, carbonyl, hydroxy, ether, ester, halogen, —CF₃ or a combination thereof;
a compound selected from sodium selenite, selenium dioxide, sodium selenide, selenious acid or a combination thereof; and
a pharmaceutically acceptable carrier.

In some embodiments, the compound of Formula II can be

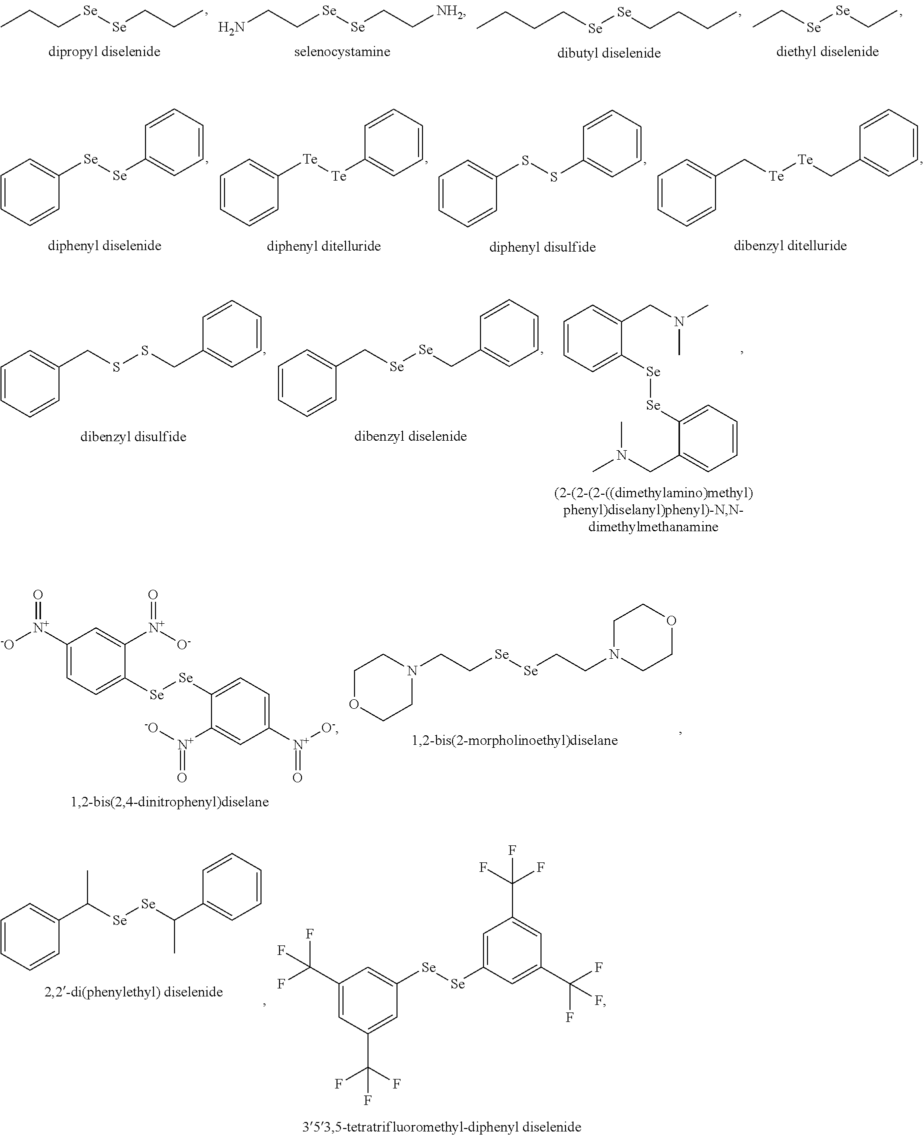

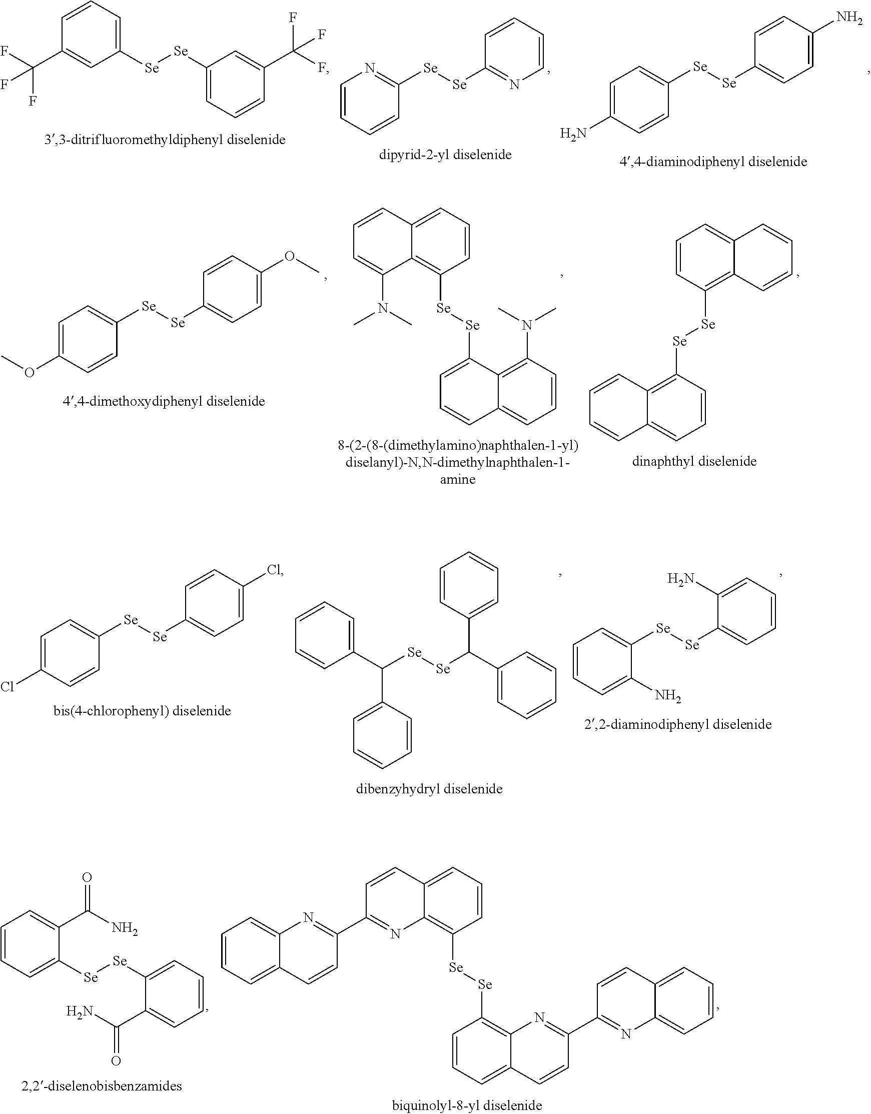

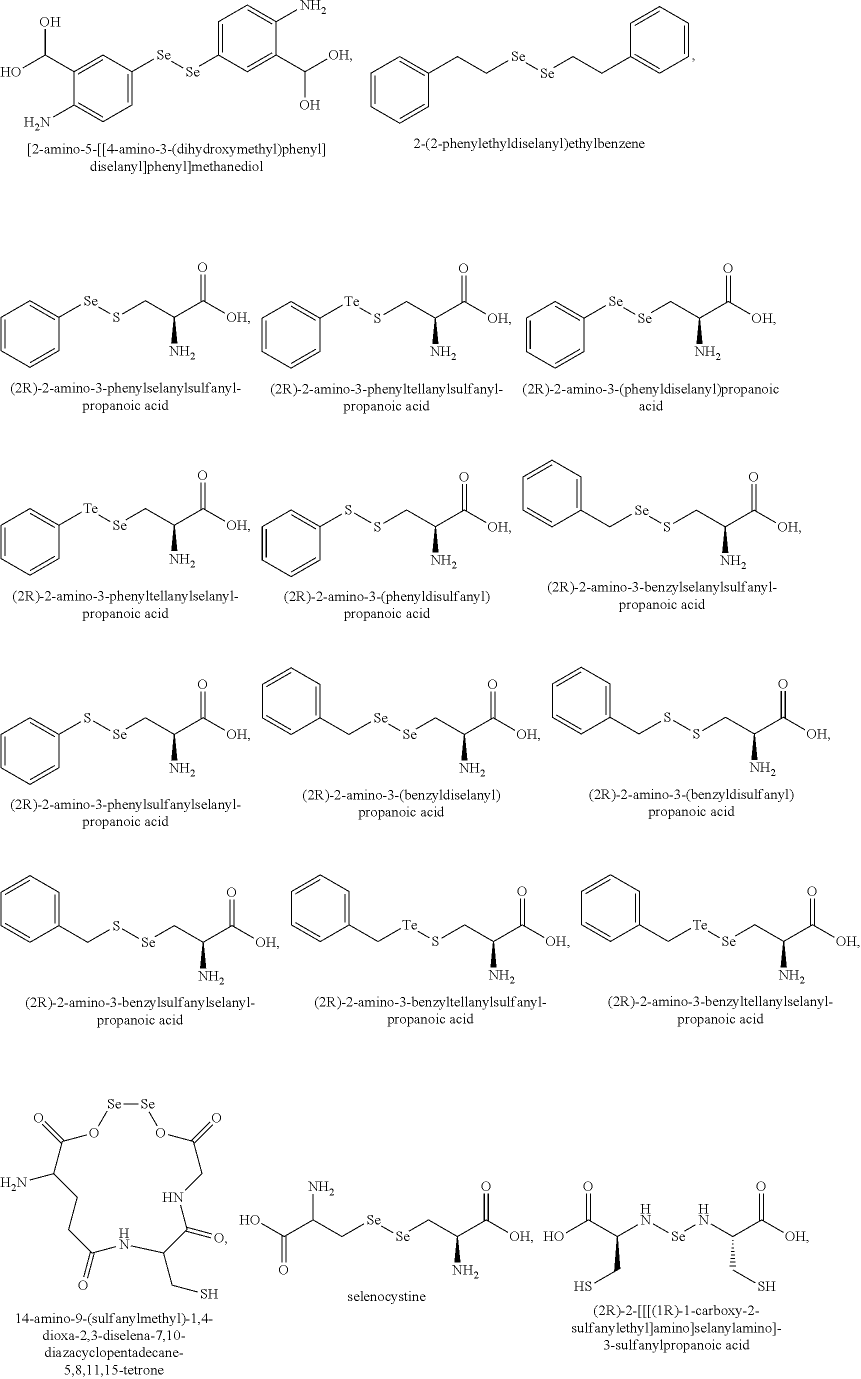

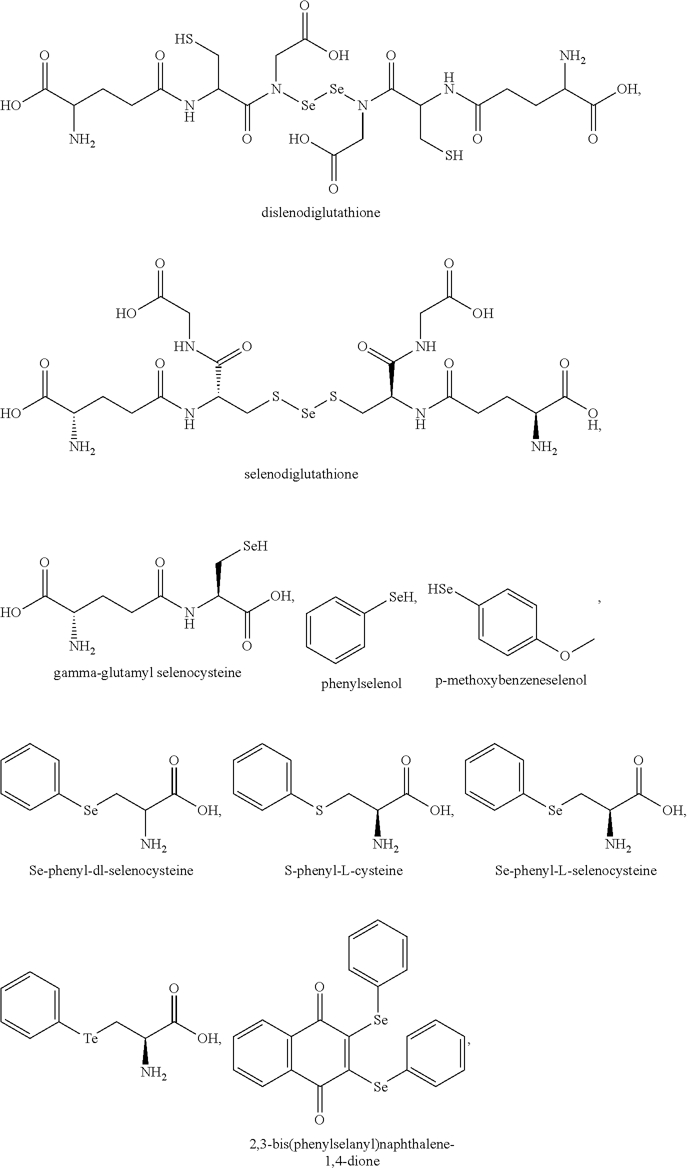

a pharmaceutically acceptable salt thereof, or a combination thereof.

In some embodiments, the one or more compounds of Formula II is diphenyl diselenide, phenylselenol, selenocystine, dibenzyl diselenide, or a combination thereof. In some embodiments, the pharmaceutical compositions for the treatment of coronaviruses can be used in combination with methionine, cystine, cysteine, N-acetyl cysteine, S-adenosyl-L-methionine, gamma-glutamyl cysteine, cystathionine, vitamin C, or a combination thereof. In some embodiments, the composition for the treatment of coronaviruses can be used in combination with methionine, cystine, or a combination thereof.

The disclosed compounds can be used therapeutically in combination with a pharmaceutically acceptable carrier. The carrier would naturally be selected to minimize any degradation of the active ingredient and to minimize any adverse side effects in the subject, as would be well known to one of skill in the art.

The disclosed compositions may be in solution, suspension, incorporated into microparticles, liposomes, or cells, or formed into tablets, gels, or suppositories. Suitable carriers and their formulations are described in Remington: The Science and Practice of Pharmacy (22^{nd} ed.) eds. Loyd V. Allen, Jr., et al., Pharmaceutical Press, 2012. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of vaccines to humans, including solutions such as sterile water, saline, and buffered solutions at physiological pH. Pharmaceutical compositions may include carriers, thickeners, diluents, buffers, preservatives, surface active agents and the like in addition to the vaccine. Pharmaceutical compositions may also include one or more active ingredients such as antimicrobial agents, anti-inflammatory agents, anesthetics, and the like.

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

### Methods of Use

The present disclosure provides methods for treating or ameliorating at least one symptom or indication of a disease including administering to a subject in need thereof a pharmaceutical composition including a therapeutically effective amount of a compound described herein. In some embodiments, the disease is cancer. In some embodiments, the disease is a disease in which activation of NFE2L2 or ATF4 leads to the amelioration of at least one symptom or indication of the disease. In some embodiments, the methods for treating or ameliorating at least one symptom or indication of cancer include administering to a subject in need thereof a pharmaceutical composition including a therapeutically effective amount of a compound described herein. In some embodiments, the methods for treating or ameliorating at least one symptom or indication of cancer include administering to a subject in need thereof a pharmaceutical composition including a therapeutically effective amount of a compound of Formula I. In some embodiments, provided is a method for treating cancer including administering to a subject in need thereof a therapeutically effective amount of the pharmaceutical composition including a therapeutically effective amount of a compound described herein. In some embodiments, provided is a method for treating cancer including administering to a subject in need thereof a therapeutically effective amount of the pharmaceutical composition comprising a compound of Formula I. In specific embodiments, the present disclosure provides methods for treating or ameliorating at least one symptom or indication or inhibiting the growth of tumors including administering to a subject in need thereof a pharmaceutical composition including a therapeutically effective amount of a compound described herein. In specific embodiments, the present disclosure provides methods for treating or ameliorating at least one symptom or indication or inhibiting the growth of tumors including administering to a subject in need thereof a pharmaceutical composition including a therapeutically effective amount of a compound of Formula I. In certain embodiments, the present disclosure provides a method for activating the overexpression of NFE2L2 or ATF4 transcription factors including administering to a subject in need thereof a pharmaceutical composition including a therapeutically effective amount of a compound described herein in a subject in need thereof. In some embodiments, also provided are methods for increasing in a subject in need thereof the expression of human cystine/glutamate transporter protein encoded by human SLC7A11 gene. In some embodiments, also provided are methods for increasing in a subject in need thereof the concentration of human cystine/glutamate transporter protein.

In some embodiments, the subject in need thereof can have a lymphoma, myeloid leukemia, bladder cancer, brain cancer including glioblastoma, head and neck cancer, kidney cancer, lung cancer, myeloma, melanoma, ovarian cancer, cervical cancer, bone cancer, thyroid cancer, adrenal gland cancer, cholangiocarcinoma, pancreatic cancer, prostate cancer, skin cancer, liver cancer, testicular cancer, colon cancer, or breast cancer. In some embodiments, the subject in need thereof can have liver cancer.

In certain embodiments, the cancer or tumor can be a solid tumor or malignancy. The methods described herein can cause a therapeutic injury resulting in the reduction of at least one of surface area, the depth, and the amount of the tissue affected by the cancerous condition. In certain embodiments, the compounds and compositions can be used in the treatment of cancer of the bile duct, bone, bladder, head and neck, kidney, liver, gastrointestinal tissue, esophagus, ovary, endometrium, pancreas, skin, testes, thyroid, uterus, cervix and vulva, and of leukemias (including ALL and CML), multiple myeloma and lymphomas. In specific embodiments, the compounds and compositions can be used in the treatment of lung cancer, anal cancer, colorectal cancer, prostate cancer, melanoma, renal cancer, skin cancer, testicular cancer, ovarian cancer, breast cancer, endometrial cancer, kidney cancer, gastric cancer, sarcomas, bladder cancer, brain cancer, cervical cancer, gastrointestinal cancer, genitourinary cancer, esophageal cancer, pancreatic cancer, colon cancer, liver cancer, uterine cancer, bone cancer, stomach cancer, salivary gland cancer, head and neck cancers, myeloid leukemia, adrenal gland cancer, tumors of the central nervous system and their metastases, and also for the treatment of glioblastomas and myeloma. In some specific embodiments, the cancer is lung cancer.

In some embodiments, compounds and compositions disclosed herein could be used in the clinic either as a single agent by itself, in combination with radiation, additional chemotherapy agents, or in combination with both radiation and an additional chemotherapy agent. Such chemotherapy agent can include one or more of the following categories of anti-tumour agents:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; venetoclax; and the agent alpha-interferon;
(ii) cytostatic agents such as anti-estrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents that inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbbl antibody cetuximab), farnesyl transferase inhibitors, tyrosine kinase inhibitors and serine/threonine kinase inhibitors, for example inhibitors of the epidermal growth factor family (for example EGFR family tyrosine kinase inhibitors such as: N-(3-chloro-4-fluorophenyl)-7-methoxy-6-(3-morpholinopropoxy)quinazolin-4-a mine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors or phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS-354825) and imatinib mesylate (Gleevec™); and any agents that modify STAT signalling;
(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvP3 function and angiostatin);
(vi) vascular damaging agents such as Combretastatin A4;
(vii) antisense therapies, for example those which are directed to the targets listed above, such as an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, and approaches using the immunomodulatory drugs thalidomide and lenalidomide [Revlimid®].

Combination treatment with an additional chemotherapy agent can be achieved by way of the simultaneous, sequential or separate dosing of the individual components of the treatment. Such combination products employ the compounds or compositions of this disclosure, or pharmaceutically acceptable salts thereof, within the dosage range described hereinbefore and the other pharmaceutically-active agent within its approved dosage range.

The methods described herein are provided for treating or ameliorating at least one symptom or indication or inhibiting the growth of cancer in a subject. In certain embodiments, methods are provided for increasing the overall or progression-free survival of a patient with cancer.

Disclosed are also methods for treating, inhibiting, decreasing, reducing, ameliorating and/or preventing Zika virus, rhinovirus, influenza virus infection, human immunodeficiency virus (HIV), or cutaneous leishmaniasis, or a coronavirus infections in a subject in need thereof, the method comprising administering a therapeutically effective amount of the pharmaceutical composition including one or more compounds of Formula II, a compound selected from sodium selenite, selenium dioxide, sodium selenide, selenious acid or a combination thereof; and a pharmaceutically acceptable carrier.

In another aspect, the present disclosure provides methods for treating Zika virus, rhinovirus, influenza virus infection, human immunodeficiency virus (HIV), or cutaneous leishmaniasis, or a coronavirus infections in a subject in need thereof, the method comprising administering a therapeutically effective amount of the pharmaceutical composition including one or more compounds having Formula II, a compound selected from sodium selenite, selenium dioxide, sodium selenide, selenious acid or a combination thereof; and a pharmaceutically acceptable carrier.

In another aspect, the present disclosure provides methods for preventing the disease and/or symptoms associated with Zika virus, rhinovirus, influenza virus infection, human immunodeficiency virus (HIV), cutaneous leishmaniasis, or a coronavirus infection in a subject in need thereof, comprising administering a therapeutically effective amount of the pharmaceutical composition including one or more compounds having Formula II, a compound selected from sodium selenite, selenium dioxide, sodium selenide, selenious acid or a combination thereof; and a pharmaceutically acceptable carrier.

A “coronavirus infection” as used herein refers to an infection caused by or otherwise associated with growth of coronavirus in a subject, in the family Coronaviridae (subfamily Coronavirinae).

Coronaviruses are species of virus belonging to the subfamily Coronavirinae in the family Coronaviridae, in the order Nidovirales. Coronaviruses are enveloped viruses with a positive-sense single-stranded RNA genome and with a nucleocapsid of helical symmetry.

In one embodiment, the coronavirus infection is an infection of the upper and/or lower respiratory tract. The “upper respiratory tract” includes the mouth, nose, sinus, middle ear, throat, larynx, and trachea. The “lower respiratory tract” includes the bronchial tubes (bronchi) and the lungs (bronchi, bronchioles and alveoli), as well as the interstitial tissue of the lungs.

In another embodiment, the coronavirus infection is an infection of the gastrointestinal tract. The “gastrointestinal tract” may include any area of the canal from the mouth to the anus, including the mouth, esophagus, stomach, and intestines.

In yet another embodiment, the coronavirus infection is a renal infection.

It is understood and herein contemplated that the coronavirus infections disclosed herein can cause a pathological state associated with the coronavirus infection referred to herein as a “coronavirus disease.” In some embodiments, the coronavirus disease is selected from a common cold, pneumonia, pneumonitis, bronchitis, severe acute respiratory syndrome (SARS), coronavirus disease 2019 (COVID-2019), Middle East respiratory syndrome (MERS), sinusitis, porcine diarrhea, porcine epidemic diarrhea, avian infections bronchitis, otitis and pharyngitis. In particular embodiments, the coronavirus infection is a common cold. In particular embodiments, the coronavirus infection is selected from SARS, COVID-19, and MERS. In a particular embodiment, the coronavirus infection is COVID-19. In another particular embodiment, the coronavirus infection is IBV, PorCoV HKU15, or PEDV.

Other indications associated with coronavirus infections are described in Gralinski & Baric, 2015, J. Pathol. 235:185-195 and Cavanagh, 2005, “Coronaviridae: a review of coronavirus and toroviruses”, Coronaviruses with Special Emphasis on First Insights Concerning SARS 1, ed. By A. Schmidt, M. H. Wolff and O. Weber, Birkhauser Verlag Baser, Switzerland, each of which is incorporated herein by reference in their entirety.

The coronavirus causing the infection may be selected from an alphacoronavirus, a betacoronavirus, a gammacoronavirus, or a deltacoronavirus.

Representative examples of alphacoronaviruses include, but are not limited to, a colacovirus (e.g., Bat coronavirus CDPHE15), a decacovirus (e.g., Bat coronavirus HKU10, Rhinolophus ferrumequinum alphacoronavirus Hub-2013), a duvinacovirus (e.g., Human coronavirus 229E), a luchacovirus (e.g., Lucheng Rn rat coronavirus), a minacovirus (e.g., Ferret coronavirus, Mink coronavirus 1), a minunacovirus (e.g., Miniopterus bat coronavirus 1, Miniopterus bat coronavirus HKU8), a myotacovirus (e.g., Myotis rickettii alphacoronavirus Sax-2011), a nyctacovirus (e.g., Nyctalus velutinus alphacoronavirus SC-2013), a pedacovirus (e.g., Porcine epidemic diarrhea virus (PEDV), Scotophilus bat coronavirus 512), a rhinacovirus (e.g., Rhinolophus bat coronavirus HKU2), a setracovirus (e.g., Human coronavirus NL63, NL63-related bat coronavirus strain BtKYNL63-9b), and a tegacovirus (e.g. Alphacoronavirus 1).

Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang20l3), a merbecovirus (e.g., Hedgehog coronavirus 1, Middle East respiratory syndrome-related coronavirus (MERS-CoV), Pipistrellus bat coronavirus HKU5, Tylonycteris bat coronavirus HKU4), a nobecovirus (e.g., Rousettus bat coronavirus GCCDC1, Rousettus bat coronavirus HKU9), a sarbecovirus (e.g., severe acute respiratory syndrome coronavirus (SARS-CoV), severe acute respiratory syndrome coronavirus 2 (SARS-CoV-2).

Representative examples of gammacoronaviruses include, but are not limited to, a cegacovirus (e.g., Beluga whale coronavirus SQ1) and an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), and a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus OC43 (HCoV-OC43), human coronavirus HKU1 (HCoV-HKU1), Human coronavirus NL63 (HCoV-NL63), severe acute respiratory syndrome coronavirus (SARS-CoV), severe acute respiratory syndrome coronavirus 2 (SARS-CoV-2), and Middle East respiratory syndrome-related coronavirus (MERS-CoV).

In one embodiment, a method is provided for treating coronavirus disease 2019 (COVID-2019) including administering a therapeutically effective amount of the pharmaceutical composition having one or more compounds having Formula II, a compound selected from sodium selenite, selenium dioxide, sodium selenide, selenious acid or a combination thereof; and a pharmaceutically acceptable carrier.

In one embodiment, a method is provided for preventing coronavirus disease 2019 (COVID-2019) including administering a therapeutically effective amount of the pharmaceutical composition having one or more compounds having Formula II, a compound selected from sodium selenite, selenium dioxide, sodium selenide, selenious acid or a combination thereof; and a pharmaceutically acceptable carrier.

In another embodiment, a method is provided for inhibiting, decreasing, reducing, ameliorating and/or preventing one or more symptoms associated with coronavirus disease 2019 (COVID-2019) including administering a therapeutically effective amount of the pharmaceutical composition having one or more compounds having Formula II, a compound selected from sodium selenite, selenium dioxide, sodium selenide, selenious acid or a combination thereof; and a pharmaceutically acceptable carrier.

Another embodiment is a method of inhibiting viral protease including administering a pharmaceutical composition having one or more compounds having Formula II, a compound selected from sodium selenite, selenium dioxide, sodium selenide, selenious acid or a combination thereof; and a pharmaceutically acceptable carrier.

In one embodiment, a method is provided for inhibiting the main viral protease responsible for cleaving the polyprotein of a coronavirus into active components in a coronavirus-infected cell, the method including contacting the cell with a therapeutically effective amount of pharmaceutical composition having one or more compounds having Formula II, a compound selected from sodium selenite, selenium dioxide, sodium selenide, selenious acid or a combination thereof; and a pharmaceutically acceptable carrier. In some embodiments, the coronavirus is SARS-CoV-2. In some embodiments, the cell is a human cell. In some embodiments, the main viral protease is selected from SARS-CoV-2 main protease, SARS-CoV main protease, and MERS-CoV main protease. In some embodiments, the main viral protease is a SARS-CoV-2 main protease inhibitor.

In another embodiment, a method is provided for reducing viral load of a coronavirus in a coronavirus-infected cell, the method comprising contacting the cell with a therapeutically effective amount of the pharmaceutical composition having one or more compounds having Formula II, a compound selected from sodium selenite, selenium dioxide, sodium selenide, selenious acid or a combination thereof; and a pharmaceutically acceptable carrier. In some embodiments, the coronavirus is SARS-CoV-2. In some embodiments, the cell is a human cell.

In another embodiment, a method is provided for reducing inflammation in a tissue of a subject infected with a coronavirus, the method comprising administering a therapeutically effective amount of the pharmaceutical composition having one or more compounds having Formula II, a compound selected from sodium selenite, selenium dioxide, sodium selenide, selenious acid or a combination thereof; and a pharmaceutically acceptable carrier. In some embodiments, the coronavirus is SARS-CoV-2. In some embodiments, the tissue is lung tissue.

Another embodiment is a method of treating hyperinflammation, hypercytokinemia, inflammatory immune response, ischemia-reperfusion injury, sepsis or combinations thereof arising out of an infection, the method including administering the pharmaceutical composition having one or more compounds having Formula II, a compound selected from sodium selenite, selenium dioxide, sodium selenide, selenious acid or a combination thereof; and a pharmaceutically acceptable carrier. In some embodiments, the hyperinflammation can be associated with cytokine storm leading to macrophage activation syndrome.

In some embodiments, method of treating hyperinflammation arising out of an infection, the method including administering the pharmaceutical composition having one or more compounds having Formula II, a compound selected from sodium selenite, selenium dioxide, sodium selenide, selenious acid or a combination thereof; and a pharmaceutically acceptable carrier. In some embodiments, the infection can be a viral or bacterial infection. In some embodiments, a method of treating hyperinflammation arising out of other viral or bacterial infections, the method including administering the pharmaceutical composition having one or more compounds having Formula II, a compound selected from sodium selenite, selenium dioxide, sodium selenide, selenious acid or a combination thereof; and a pharmaceutically acceptable carrier. In some embodiments, the bacterial infection is a systemic bacterial infection that leads to sepsis. In some embodiments, the viral infection can be a coronavirus infection, Zika virus, rhinovirus, influenza virus infection, or human immunodeficiency virus (HIV).

### Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

The disclosed compositions are preferably formulated for delivery via intranasal, intramuscular, subcutaneous, parenteral, transdermal sublingual, buccal, intravenous, intradermal, intraperitoneal, oral, eye drops, or topical administration.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Parenteral administration of the disclosed compounds, if used, is generally characterized by injection. Injectables can be prepared in conventional forms, either as liquid solutions or suspensions, solid forms suitable for solution of suspension in liquid prior to injection, or as emulsions.

For an oral administration form, the disclosed compounds can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, cornstarch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the disclosed compounds may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may additionally contain other pharmaceutical auxiliaries such as surfactants, emulsifiers and stabilizers as well as a propellant.

For subcutaneous or intravenous administration, the disclosed compounds, if desired with the substances customary therefore such as solubilizers, emulsifiers or further auxiliaries are brought into solution, suspension, or emulsion. The disclosed compounds may also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

In certain embodiments, it is contemplated that compositions comprising the disclosed compounds can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na-CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release delivery systems. The disclosed compounds can be entrapped in the poly(lactide-co-glycolide) microsphere depot by a number of methods, including formation of a water-in-oil emulsion with water-borne compound and organic solvent-borne polymer (emulsion method), formation of a solid-in-oil suspension with solid compound dispersed in a solvent-based polymer solution (suspension method), or by dissolving the compound in a solvent-based polymer solution (dissolution method). One can attach poly(ethylene glycol) to compounds (PEGylation) to increase the in vivo half-life of circulating therapeutic proteins and decrease the chance of an immune response.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present disclosure.

Compositions for topical or transdermal administration include ointments, pastes, creams, lotions, gels, powders, solutions, sprays, inhalants, or patches. The active compound is admixed with an excipient and any needed preservatives or buffers as may be required.

Some of the disclosed compounds may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Compositions, as described herein, comprising an active compound and an excipient of some sort may be useful in a variety of medical and non-medical applications. For example, pharmaceutical compositions comprising an active compound and an excipient may be useful for the treatment of cancer.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the therapeutic doses of the compounds described herein can ranges from 0.01 mg/m2 to 1.0 g/m2 body surface area daily in human subjects or in animals. For example, category 1 or category 2 compounds can be administered individually, simultaneously, successively or at different time intervals with category 3, category 4 or category 5 compounds or combinations thereof. The efficacy of such combination treatments can be potentiated by strategic administration of pharmacologically accepted preparations of cystine, methionine, cysteine, N-acetyl cysteine, gamma-glutamyl cysteine, glutathione, cystathionine, vitamin C at dose ranging from 0.01 mg/m2 to 1.0 g/m2 body surface area daily or equivalent dose depending on the route of administration. For example, category 1, category 2 and category 3 molecules can be used at a specific dosing schedule in which these molecules are administered in human subjects at a constant rate of greater than 0.02 mg/m2 body surface area/hour daily or equivalent dose for 18 days, while category 4 and category 5 molecules are individually co-administered prior to or concurrently at a rate of greater than 0.05 mg/m2 body surface area/hour or equivalent dose or as a single administration of dose exceeding 2.00 mg/m2 body surface area in every 2-24 hour interval for 18 days in human subjects.

In some embodiments, the compositions as used in the methods described herein can further comprise or be administered in combination with other therapies. The composition described herein can be administered simultaneously, sequentially, or at distinct time points as part of the same therapeutic regimen.

The disclosed compounds can also be used to supplement existing treatments. Therefore, the disclosed compositions can further include (or be administered in combination with) a second compound that can ameliorate, diminishing, reversing, treating or preventing cancer in a subject. For example, the disclosed compositions can further include (or be administered in combination with) one or more chemotherapeutic agents. In a specific embodiment, the disclosed compounds can be administered with (in combination in the same composition, in combination but in separate compositions, or sequentially) approved drugs for treating cancer.

The pharmaceutical compositions and formulations disclosed herein can be administered for prophylactic and/or therapeutic treatments. In therapeutic applications, compositions are administered to a subject already having a tumor.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the condition, the severity of the condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

In some embodiments, the composition as used in the methods described herein may be administered in combination or alternation with one or more additional active agents. Representative examples additional active agents include antimicrobial agents (including antibiotics, antiviral agents and anti-fungal agents), anti-inflammatory agents (including steroids and non-steroidal anti-inflammatory agents), anti-coagulant agents, antiplatelet agents, and antiseptic agents.

Representative examples of antibiotics include amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, and vancomycin.

Representative examples of antiviral agents include, but are not limited to, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, and zidovudine.

Representative examples of anticoagulant agents include, but are not limited to, heparin, warfarin, rivaroxaban, dabigatran, apixaban, edoxaban, enoxaparin, and fondaparinux.

Representative examples of antiplatelet agents include, but are not limited to, clopidogrel, ticagrelor, prasugrel, dipyridamole, dipyridamole/aspirin, ticlopidine, and eptifibatide.

Representative examples of antifungal agents include, but are not limited to, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, and amphotericin b.

Representative examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Representative examples of non-steroidal anti-inflammatory drugs include ibuprofen, naproxen, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

Other examples of additional active agents include chloroquine, hydrochloroquine, Pyridoxal phosphate, Vitamin D, and Vitamin C

In some embodiments, the composition as used in the methods described herein may be administered in combination or alternation with one or more anticytokine or immunomodulatory agents, representative examples of which include, but are not limited to, tocilizumab, sarilumab, bevacizumab, fingolimod, imiquimod, and eculizumab.

In some embodiments, the composition as used in the methods described herein may be administered in combination or alternation with an immunoglobulin therapy.

This written description uses examples to disclose the invention, including the best mode, and also to enable any person skilled in the art to make and use the invention. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims. All citations referred herein are expressly incorporated herein by reference. It is to be expressly understood, however, that each of the figures is provided for the purpose of illustration and description only and is not intended as a definition of the limits of the present invention.

By way of non-limiting illustration, examples of certain embodiments of the present disclosure are given below.

### Examples

### Example 1

### Cytotoxicity Studies

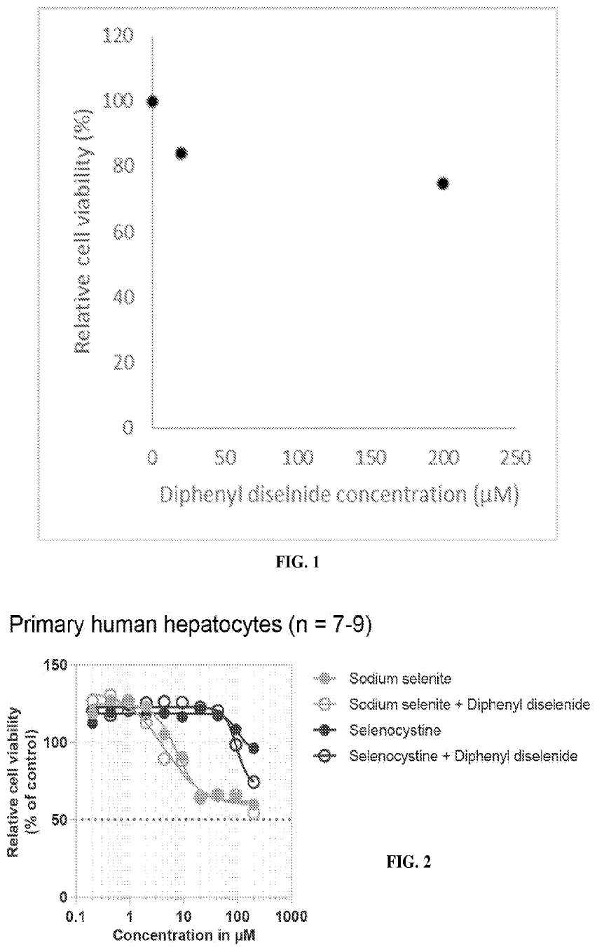

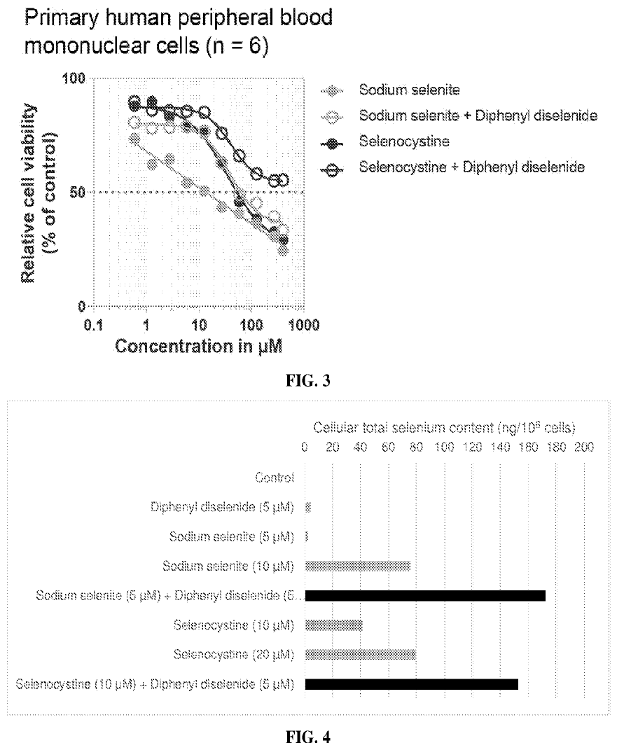

Cytotoxicity of diphenyl diselenide in different cancer cell lines and in normal cells was tested and the results are shown in Table 1 and FIG. 1. The cytotoxicity of sodium selenite and selenocystine either alone or in combination with diphenyl diselenide in different cancer cell lines and in normal cells was also tested and the results are shown in Table 2, Table 5, and FIGS. 2 and 3. The cytotoxicity of sodium selenite and selenocystine either alone or in combination with other classes of compounds in different cancer cell lines was also tested and the results are shown in Tables 3 and 4.

For the cytotoxicity studies the cells were seeded in 96 well plates (BD Falcon, Durham, NC, USA) in 100 μl respective culture media. After 24 h, media was replaced with the respective compounds at their indicated final concentrations for another 24 h. Nine tenth volume of the media was exchanged at the end of the exposure period following which cytotoxicity was measured using WST-1 reagent. Cells were incubated at 37° C. for 30-60 min depending on the cell types and absorbance was measured at 430 nm and 630 nm (background absorbance) using a spectrophotometer (PowerWave HT, BioTek, VT, USA).

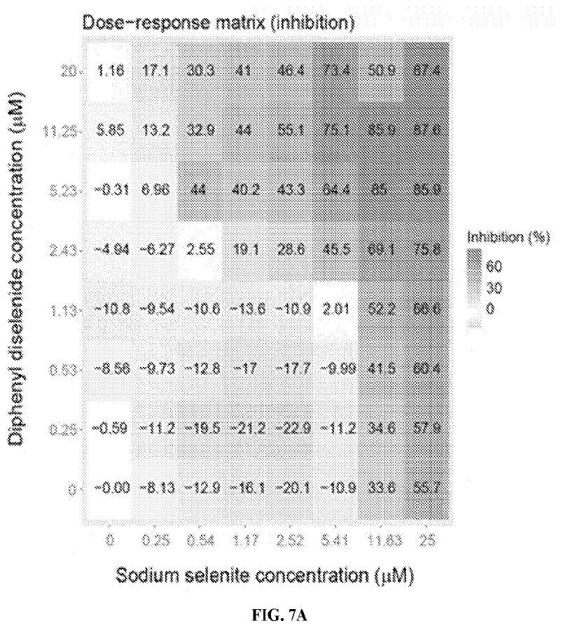

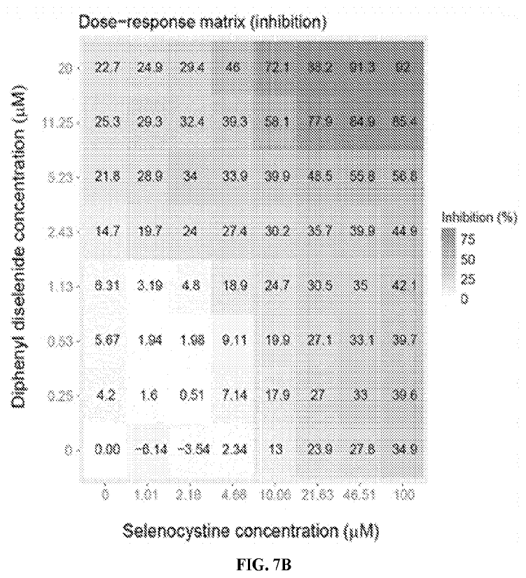

The results for the studies are shown in FIGS. 1-7 and Tables 1-5. FIG. 1 and FIG. 7 demonstrate that diphenyl diselenide is non-toxic to normal human cells and in mice, respectively at a very high dose. Table 1 show that key cytotoxicity modifying compounds exert limited cytotoxic effects to cancer cells.

Table 1 show cytotoxicity of diphenyl diselenide and functionally-related other compounds in different cancer cell lines. For some of the compounds, cytotoxicity is too little to accurately calculate IC₅₀ values (concentration at which 50% of cell population were dead at termination of the treatment). In these cases, dose approximation is made which is close to 50% relative cell viability. The tested concentrations varied among the compounds and ranged from 0-400 μM.

TABLE 1

| | | IC50 values (μM) | | | | | | |
|---|---|---|---|---|---|---|---|---|
| Cancer | Cell | Diphenyl | Tert-butyl | Phenyl | | Dimethyl | | |
| type | line | diselenide | hydroxyquinone | disulfide | Sulforaphen | fumarate | Curcumin | Tunicamycin |
| Leukemia | HL60 | 38.35 | >25 | >40 | >20 | >40 | >20 | |
| Leukemia | NB4 | 39.46 | >20 | >20 | >20 | >40 | >20 | |
| Lung | H1299 | 26.16 | >100 | | | | | >20 |
| Lung | H661 | 47.18 | >40 | >20 | >10 | >40 | >20 | |
| Lung | A549 | 91.08 | >20 | >40 | >10 | >40 | >20 | |
| Breast | MCF7 | 42.69 | >60 | | | | | >5 |

Table 2 shows an exemplary and unexpected efficacy of sodium selenite and selenocystine in killing cancer cells when combined with diphenyl diselenide which itself is non-toxic at the specified dose (5 μM). Plasma selenium concentration in normal human population ranges from 0.88-1.65 μM (about 60-70% of which is incorporated into selenoproteins the form of selenocysteine) as total selenium which is equivalent to 1.92-3.61 μM sodium selenite. Herein it is shown that certain cancer cell types (e.g., leukemic cells) can be sensitized at low micromolar to nanomolar concentration of sodium selenite or selenocystine when used in combination with diphenyl diselenide. Table 2 show the cytotoxicity of sodium selenite and selenocystine either alone or in combination with diphenyl diselenide in different cancer cell lines. In certain instances, cytotoxicity was not enough to calculate IC₅₀ values at dose up to 200 μM. N/A means either data are not available or IC₅₀ values cannot be calculated.

TABLE 2

| | | Calculated IC50 values (μM) for selenite in the presence of 5 μM diphenyl | | | | | |
|---|---|---|---|---|---|---|---|
| | | diselenide; n = 3-17, each with 3 technical replicates in each experiment | | | | | |
| Cancer | Cell | Sodium | Sodium selenite + | Fold-increase | | Selenocystine + | Fold-increase |
| type | line | selenite | Diphenyl diselenide | in | Selenocystine | Diphenyl | in |
| Lung | A549 | 2.86 | 1.204 | 2.38 | 8.7 | 1.13 | 7.70 |
| | H661 | 53.05 | 0.758 | 69.99 | 55.7 | 1.43 | 38.95 |
| | H1299 | 5.25 | 0.351 | 14.96 | 22.7 | 12.49 | 1.82 |
| | U1906E | 2.44 | 0.782 | 3.12 | 2.18 | 2.76 | 0.79 |
| | U1906L | 1.58 | 0.521 | 3.03 | 17.77 | 0.54 | 32.91 |
| Leukemia | NB4 | 24.61 | 0.32 | 76.91 | 44.14 | 0.68 | 64.91 |
| | HL60 | 15.95 | 0.19 | 83.95 | 22.58 | 0.22 | 102.64 |
| | THP-1 | 0.79 | 0.49 | 1.61 | 0.92 | 0.86 | 1.07 |
| | Kasumi 6 | 25.73 | 0.24 | 107.21 | N/A | N/A | N/A |
| | AML193 | 6.78 | 0.41 | 16.54 | 47.01 | 13.56 | 3.47 |
| Liver | HepG2 | 7.58 | 0.18 | 42.11 | 12.44 | 2.04 | 6.10 |
| | Hep3B | 1.65 | 0.11 | 15.00 | 74.73 | 50.63 | 1.48 |
| | HUH7 | 3.53 | 0.11 | 32.09 | 92.74 | 1.202 | 77.15 |
| Neuroblastom | SHSY5-SY | 40.26 | 0.1 | 402.60 | 114.96 | 57.87 | 1.99 |
| Glioblastoma | SNB-19 | 5.35 | 2.46 | 2.17 | 78.72 | 19.84 | 3.97 |
| | 8-MGBA | 4.29 | 1.4 | 3.06 | 49.69 | 13.99 | 3.55 |
| Colon | LS174T | 3.6 | 1.6 | 2.25 | 3.88 | 2.15 | 1.80 |
| Cervical | HeLa | 0.64 | 0.13 | 4.92 | 5.78 | 5.01 | 1.15 |
| Breast | MCF-7 | 8.55 | 2.24 | 3.82 | 34.88 | 4.98 | 7.00 |
| Pancreas | PANC-1 | 7.87 | 1.44 | 5.47 | 18.52 | 3.41 | 5.43 |
| Melanoma | A375 | 6.14 | 1.62 | 3.79 | 8.6 | 0.97 | 8.87 |
| Ovarian | IGROV1 | 5.08 | 1.21 | 4.20 | Non-toxic* | 64.12 | N/A |
| | OV2008 | 3.7 | 0.64 | 5.78 | 6.98 | 5.18 | 1.35 |

| | |
|---|---|
| Range: 1.61-402.6 fold | Range: 0.79-102.6 fold |

TABLE 3
cytotoxicity of sodium selenite either alone or in combination with other classes of compounds in different cancer cell lines.

| | | Calculated IC50 values of sodium selenite (μM) in the presence of the other | | | | | | | |
|---|---|---|---|---|---|---|---|---|---|
| | | compounds; n = 3-4, each with 3 technical replicates in each experiment | | | | | | | |
| | | | Sodium | Sodium | | Sodium selenite + | Sodium | Sodium selenite + | Sodium selenite + |
| Cancer | Cell | Sodium | selenite + | selenite + | Sodium selenite + | Tert-butyl | selenite + | Dibenzyl | Dibenzyl |
| type | line | selenite | Sulphoraphane | Curcumin | Phenyl disulfide | hydroxyquinone | Tunicamycin | diselenide | ditelluride |
| Lung | H661 | 53.05 | 39.04 | 42.80 | 10.55 | | | | |
| Lung | H1299 | 5.25 | | | | 0.43 | 4.03 | | |
| Leukemia | NB4 | 24.61 | 5.81 | 12.27 | 5.59 | | | | |
| Leukemia | HL60 | 15.95 | | | 10.38 | | | | |
| Breast | MCF-7 | 8.55 | | | | 7.01 | 5.39 | 2.43 | 17.72 |

TABLE 4
cytotoxicity of selenocystine either alone or in combination with other classes of compounds in different cancer cell lines.

| | | Calculated IC50 values of selenocystine (μM) in the presence of the other | | | | | | | |
|---|---|---|---|---|---|---|---|---|---|
| | | compounds; n = 3-4, each with 3 technical replicates in each experiment | | | | | | | |
| | | | | | Seleno- | Seleno- | | Seleno- | Seleno- |
| | | | Seleno- | Seleno- | cystine + | cystine + | Seleno- | cystine + | cystine + |
| Cancer | Cell | Seleno- | cystine + | cystine + | Phenyl | Tert-butyl | cystine + | Dibenzyl | Dibenzyl |
| type | line | cystine | Sulphoraphane | Curcumin | disulfide | hydroxyquinone | Tunicamycin | diselenide | ditelluride |
| Lung | A549 | 8.70 | 5.66 | 5.80 | 4.62 | | | | |
| Lung | H1299 | 22.7 | | | | 9.47 | 17.71 | | |
| Leukemia | NB4 | 44.14 | 17.44 | 22.64 | 9.93 | | | | |
| Leukemia | HL60 | 22.58 | 19.04 | 14.07 | 14.78 | | | | |
| Breast | MCF-7 | 34.88 | | | | 1.34 | | 14.05 | 1.24 |

TABLE 5
cytotoxicity of sodium selenite and selenocystine either alone or in combination with diphenyl
diselenide in normal human cells. Calculated 24 hours IC₅₀ values are extrapolated in certain
instances, as 50% cell death could not be achieved at the highest test concentration.

| | | Calculated IC50 values (μM) for selenite and selenocystine in the presence of 5 μM | | | | | |
|---|---|---|---|---|---|---|---|
| | | diphenyl diselenide; n = 6-9, each with 3 technical replicates in each experiment | | | | | |
| | | | Sodium selenite + | | | Selenocystine + | |
| Tissue of | Cell | Sodium | Diphenyl | Fold-increase | | Diphenyl | Fold-increase |
| origin | types | selenite | diselenide | in cytotoxicity | Selenocystine | diselenide | in cytotoxicity |
| Liver | Primary Human | 202 | 187 | 1.08 | 300 | 248 | 1.21 |
| | hepatocytes | | | | | | |
| Blood | Primary peripheral | 35 | 39.69 | 0.88 | 35.41 | 41.16 | 0.86 |
| | blood | | | | | | |
| | mononuclear cells | | | | | | |

Earlier clinical studies have shown that humans have very high tolerance to sodium selenite following high dose intravenous administration without any demonstrated long-term side effects. It is possible to reach a plasma concentration of >20 μM of sodium selenite equivalent while the cancer chemotherapeutic regimen is thought to be below 10 μM. Henceforth, the lack of efficiency of sodium selenite treatment in cancer patients can be attributed to limited selenium delivery to the target cells. Diphenyl diselenide (possibly similar classes of compounds belonging to category 3, 4 and category 5) dramatically increases selenium delivery to the cells. Such combined treatments have very limited cytotoxic effects on normal cells as demonstrated in Table 5 and FIGS. 2-3.

Diphenyl Diselenide Dramatically Increases Selenium Uptake from Sodium Selenite or Selenocystine.

H1299 cells were treated with the indicated concentrations of different selenium compounds either alone or in combinations for 6 h. At the end of the treatment, cells were washed 3-times with cold PBS and collected. Cell pellets were digested and the total selenium concentration was measured using ICP-MS. The results show that diphenyl diselenide dramatically increases selenium uptake from sodium selenite or selenocystine as shown in FIG. 4. Increased selenium delivery to the cells by diphenyl diselenide warrants increased cytotoxicity in cancer cells, as outlined before.

### Supply of Cystine is Important for Selenite Cytotoxicity.

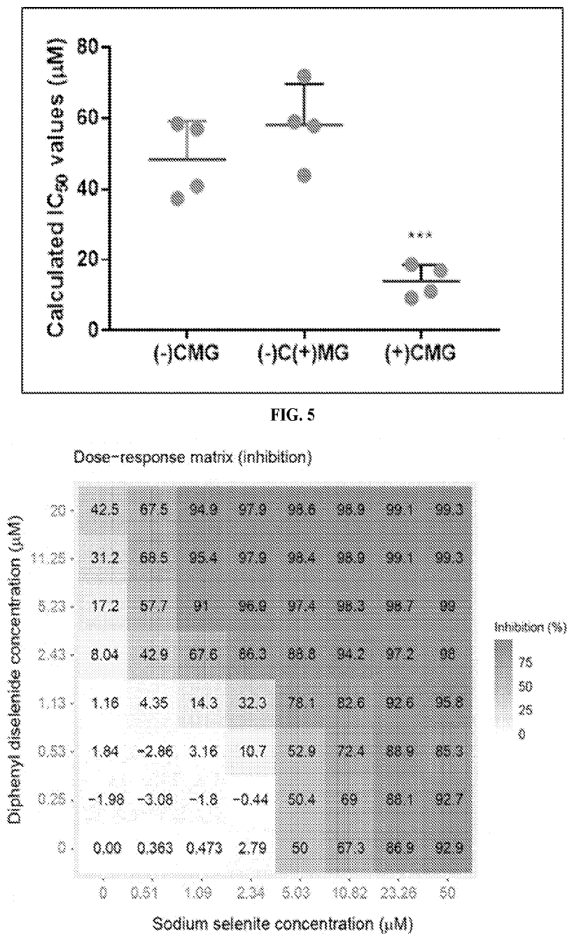

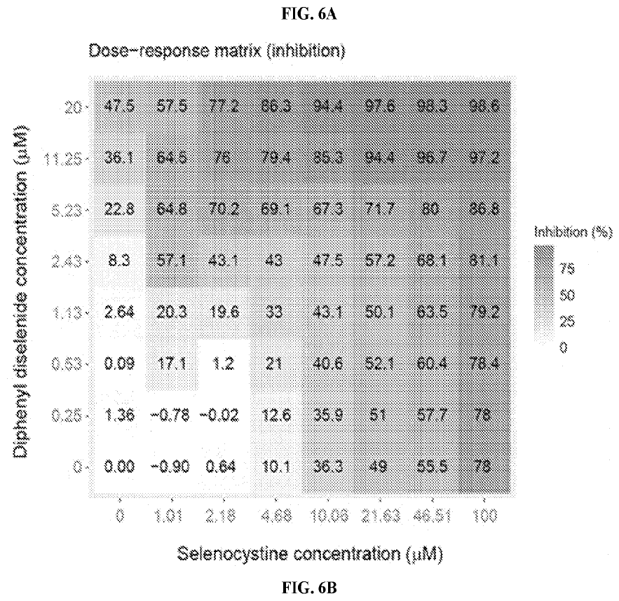

HL60 cells were cultured either in the presence or absence of cystine, methionine, and glutamine for 24 h. Subsequently, these cells were treated with different concentrations of selenite for another 24 h. Cytotoxicity was measured using WST-1 assay. The results show in FIG. 5 that the supply of cystine is important for selenite cytotoxicity in HL60. Additionally, FIGS. 6 and 7 show that combinations of either sodium selenite or selenocystine with diphenyl diselenide lead to synergistic cytotoxic effects in HL60 and H1299 cell lines which is unprecedented and not known before.

### In Vivo Studies

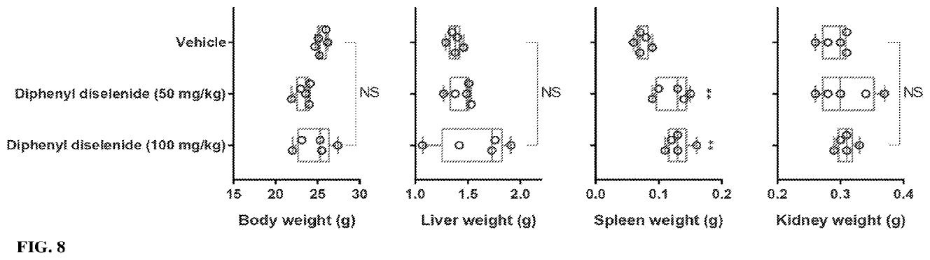

Black mice (C57BJ/6) mice were treated perorally every second day with either vehicle (canola oil) or diphenyl diselenide at two different dose levels (50 and 100 mg/kg body weight) for 3 weeks. Final body weight and weight of the major organs were measured at necropsy. No demonstrated toxic effects could be observed except that spleen weight were higher in the diphenyl diselenide treated groups. However, no abnormal histological features of spleen was noted in the diphenyl diselenide treated groups (see below), suggesting possible pleotropic effects (see FIG. 8).

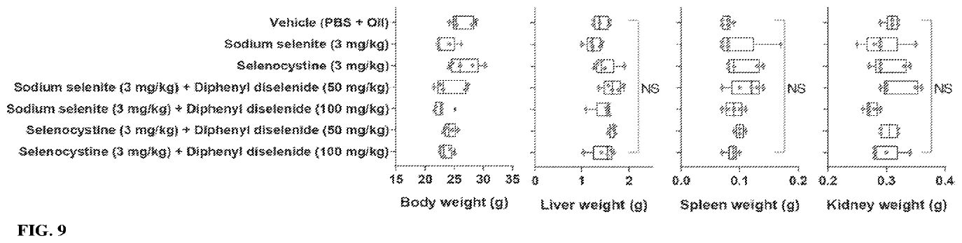

Black mice (C57BJ/6) mice were treated with vehicle (PBS i.p. and canola oil p.o.), sodium selenite (3 mg/kg body weight), selenocystine (3 mg/kg body weight), or in combinations with two different doses of diphenyl diselenide (50 and 100 mg/kg body weight) for 3 weeks. Final body weight and weight of the major organs were measured at necropsy. No demonstrated toxic effects could be observed except that lower body weight was observed in the sodium selenite+100 mg/kg diphenyl diselenide treated group. Findings from these studies demonstrate the mice can tolerate very high levels of diphenyl diselenide either alone or in combinations with either sodium selenite or selenocystine (see FIG. 9).

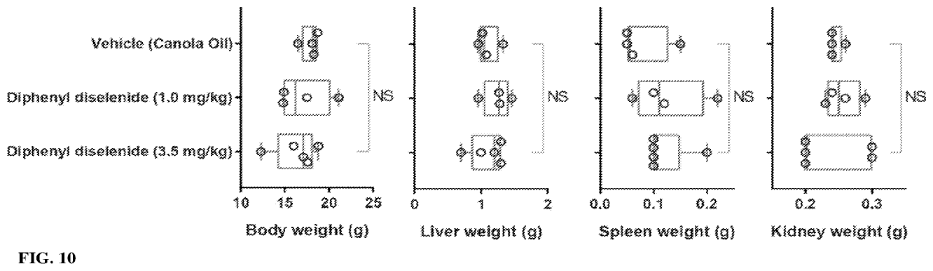

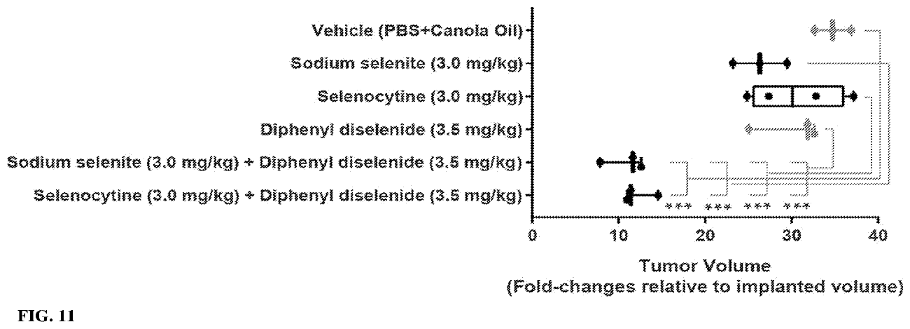

SCID mice were treated perorally every second day with either vehicle (canola oil) or diphenyl diselenide at two different dose levels (1.0 and 3.5 mg/kg body weight) for 3 weeks. Final body weight and weight of the major organs were measured at necropsy. No demonstrated toxic effects could be observed in all the treatment groups. Note that at low doses of diphenyl diselenide, splenomegaly effect is not observed (see FIG. 10)

SCID mice were primary transplanted with HL60 cells (human acute myeloid leukemia cells) in Matrigel. When palpable, secondary transplantation was carried out from the excised tumors from the primary transplanted mice. Seven days post transplantation, secondary transplanted mice were treated with vehicle (PBS i.p. and canola oil p.o.), sodium selenite (3 mg/kg body weight, i.p.), selenocystine (3 mg/kg body weight, i.p.), or in combinations with diphenyl diselenide (3.5 mg/kg body weight, p.o.) 3 times a week (Monday, Wednesday, and Friday) for 17 days. The results shown in FIG. 11 demonstrated that no toxic effects could be observed in any of the treatment groups. Note that the combined treatments of diphenyl diselenide with either sodium selenite or selenocystine leads to synergistic antitumor effects, as was shown in the in vitro studies. No significant changes in the body weight or organ weight were found at the termination of the experiment.

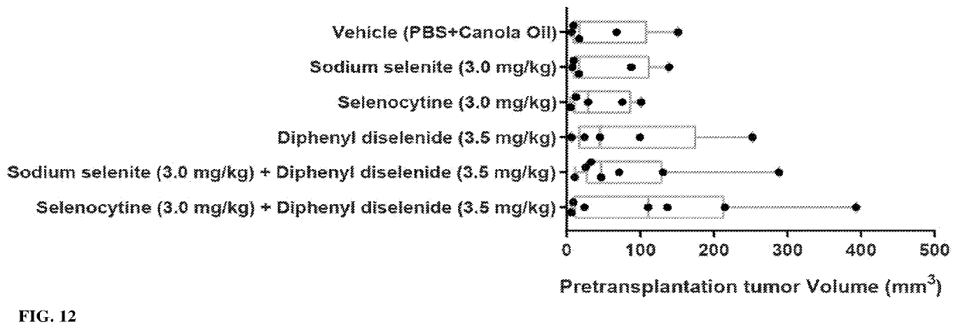

Secondary transplant volume differed among the treatment groups. Although not statistically different, when transplant volume exceeded >100 mm³, tumor growth was very fast in secondary transplanted mice and these mice had to be sacrificed before the stipulated endpoint (see FIG. 12).

SLC7A11 mRNA Expression Following Treatment with the Indicated Compounds.

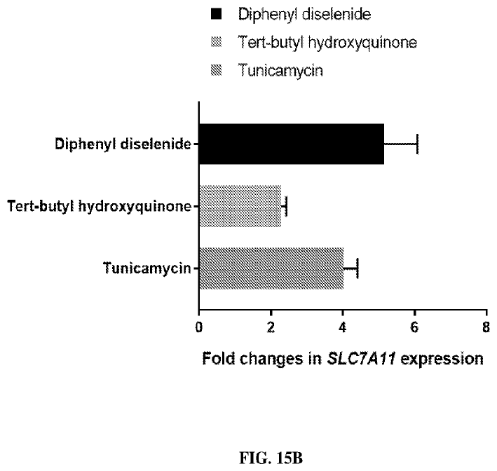

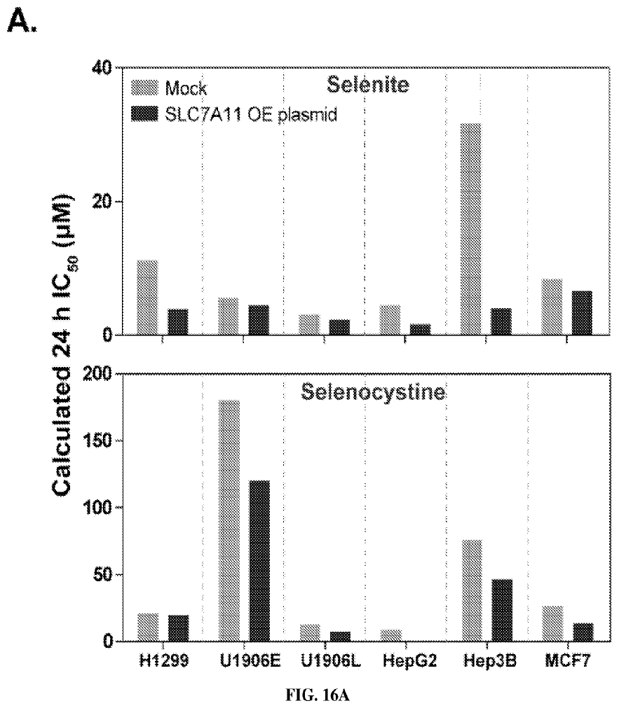

Cells were harvested at 6 h following treatment with the pharmacological compounds. Total RNA was isolated using RNeasy plus mini kit (Qiagen, Hilden, Germany) according to the manufacturer's instruction. Isolated RNAs were treated with Turbo-DNA free kit (Invitrogen, Grand Island, NY, USA) to eliminate gDNA contaminations. RNA quality was measured using NanoDrop spectrophotometer ND-1000 (Thermo Scientific). 2 μg of RNA was subjected to reverse transcription using the Ominscript RT kit (Qiagen, Hilden, Germany). Gene expression was analyzed by amplifying 1 ng of complementary DNA using the CFX96 Real-time System (Bio-Rad, Hercules, CA, USA) with iQ-SYBR Green supermix (Bio-Rad, Hercules, CA, USA) using specific primer for SLC7A11. Gene expression values for each sample were normalized to the HRPT gene. Triplicate experiments were performed and analyzed. The results for the studies are shown in FIG. 15A-15B. The data demonstrate increased expression of SLC7A11 following treatment with these compounds.

### SLC7A11 Expression is Important for Selenite and Selenocystine Cytotoxicity.

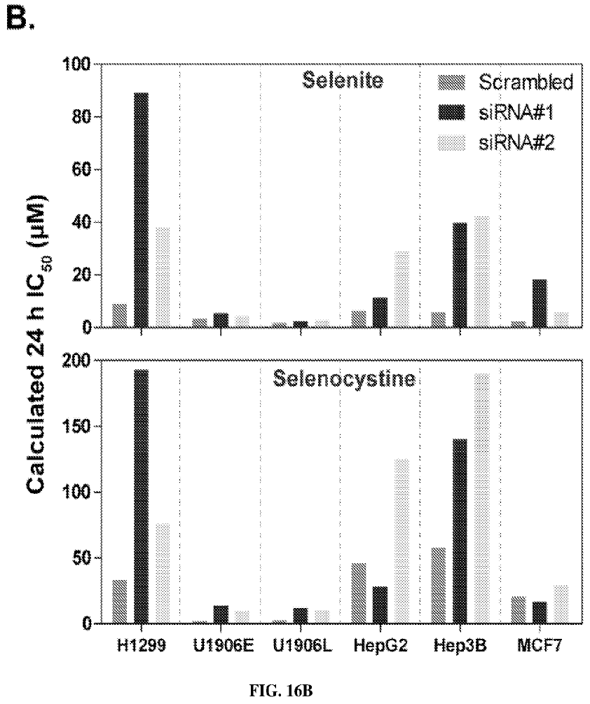

Cells were seeded into 6 well plates and grown for 24 h. Overexpression plasmid containing human full-length SLC7A11 gene was used to overexpress SLC7A11 along with empty vector. Lipofectamine 3000 (Invitrogen, Carlsbad, CA, USA) was used for transfection in the presence of 10% serum according to manufacturer's protocol. For knock-down experiments, two different SLC7A11 siRNAs (S24290 & s24289, 40 μmol each, Ambion, Life Technologies, Carlsbad, CA, USA) and control siRNA (40 μmol) (Ambion, Life Technologies, Carlsbad, CA, USA) were transfected as stated above. Twenty-four hours post-transfection, the cells were harvested and seeded in 96-well plate (Falcon, Durham, NC, USA) for subsequent treatments. Successful overexpression and knockdown of xCT (protein encoding SLC7A11 gene) in transfected cells were verified by RT-PCR. The results for the studies are shown in FIG. 16A-16B.

### NFE2L2-Inducing Activity of the Selected Compounds.

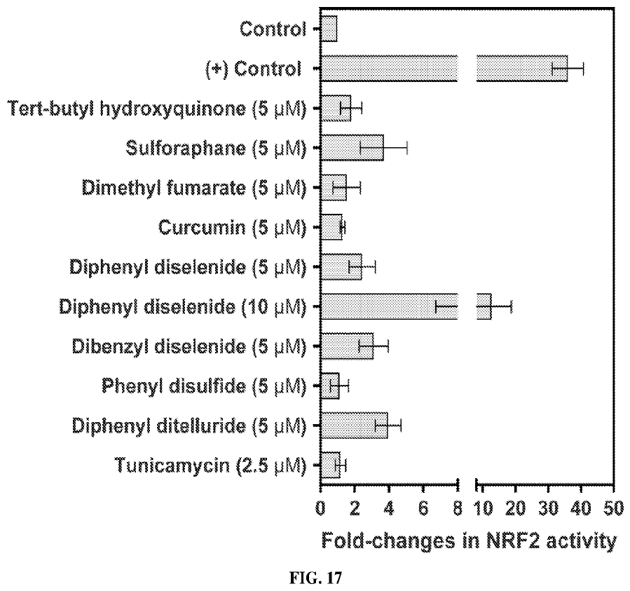

NFE2L2 reporter MCF7 cells (Signosis, Santa Clara, CA, USA) were seeded (seeding density 600 cells/mm2) in 96 well-Black plates (Thermo Scientific, Roskilde, Denmark) for overnight with DMEM (Gibco, Paisley, PA, UK) containing 10% heat-inactivated FBS (Gibco, Paisley, PA, UK). The next day, media was replaced with respective compounds as described. Twenty four hour post-treatment, media were aspirated and replaced with 1:1 ratio of PBS:Bright Glo luciferase reagent mix (Promega, Madison, WI, USA) according to manufacturer's protocol. Cells were incubated in the dark for 10 min at room temperature with gentle rocking and luciferase activity was measured using an FLx100 Luminometer (CLARIOSTAR®, Ortenbery, Germany). In parallel, the same setup was made, and the cells were used to monitor the proliferation/toxicity of individual exposed compounds. Luciferase signal is normalized to the proliferation/toxicity of the individual compounds. Tert-butyl hydroxyquinone (50 μM, Sigma-Aldrich, St. Louis, MO, USA), a known agonist for NFE2L2, was used as a positive control. The results for the studies are shown in FIG. 17. Data demonstrate transcriptional activity of NFE2L2 following treatment with these compounds.

### ATF4-Inducing Activity of the Selected Compounds.

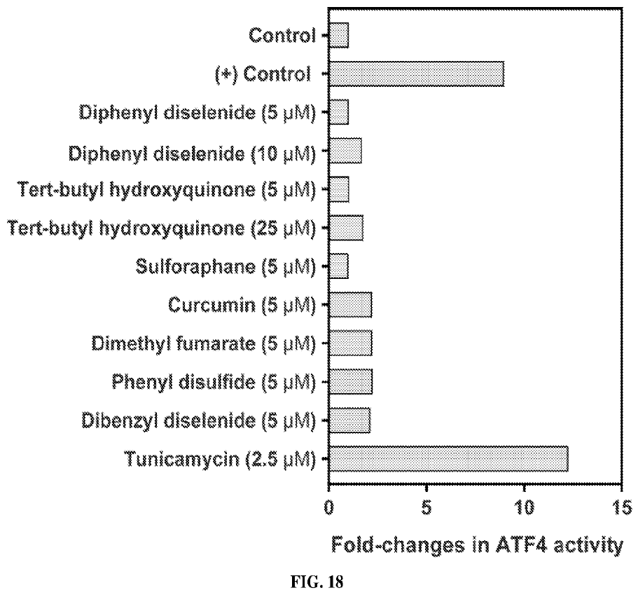

ATF4 reporter HEK 293T cells were seeded (seeding density 300 cells/mm2) in 96 well-Black plates (Thermo Scientific, Roskilde, Denmark) for overnight with DMEM (Gibco, Paisley, PA, UK) containing 10% heat-inactivated FBS (Gibco, Paisley, PA, UK). The next day, media was replaced with respective compounds as described. Twenty-four hours post-treatment, media were aspirated and replaced with 1:1 ratio of PBS:Bright Glo luciferase reagent mix (Promega, Madison, WI, USA) according to manufacturer's protocol. Cells were incubated in the dark for 10 min at room temperature with gentle rocking and luciferase activity was measured using an FLx100 Luminometer (CLARIOSTAR®, Ortenbery, Germany). In parallel, the same setup was made, and the cells were used to monitor the proliferation/toxicity of individual exposed compounds. Luciferase signal is normalized to the to the proliferation/toxicity of the individual compounds. Bortezomib (50 nM, Sigma-Aldrich, St. Louis, MO, USA), a known agonist for ATF4, was used as a positive control. The results for the studies are shown in FIG. 18. Data demonstrate transcriptional activity of ATF4 following treatment with these compounds.

### Sodium Selenite Increases the Cytotoxicity of Sorafenib, an Anticancer Drug.

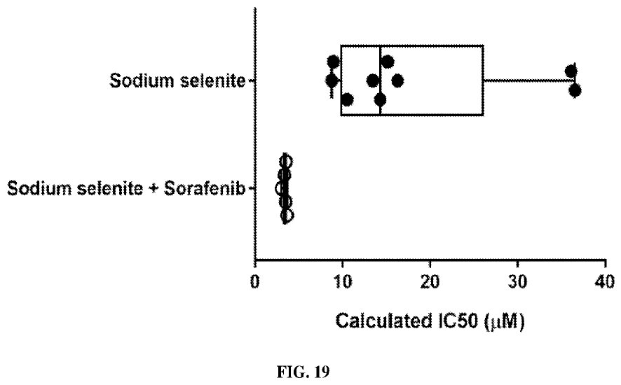

HepG2 (a human hepatocellular carcinoma cell line) cells were treated with either selenite or selenite in combination with sorafenib (5 μM) for 24 h and cytotoxicity was measured using WST-1 assay. The results for the studies are shown in FIG. 19. Data demonstrate strong cytotoxic effects of sodium selenite in a hepatocellular carcinoma cell line when used in combination with sorafenib, an anti-cancer drug.

### Results

Successful implementation of the above strategies in cancer treatment implies that such molecules are ideally non-toxic, do not have long-term side effects, and exhibit favorable pharmacokinetic properties. Described herein are classes of small molecules that have been identified such that when mammalian cells are treated with a combination of these molecules either bolster cellular antioxidant capacity or function as prooxidant molecules in a context-dependent and unforeseen manner. Such pro-oxidant capacity and subsequent cytotoxic effects on cancer cells can further be bolstered upon specific combinations of small molecules while normal cells are spared at equivalent concentrations of these experimental drugs. Such effects have been demonstrated in vitro using 23 different human cancer cell lines, ex vivo using human primary pancreatic cancer tissue slice culture, and in vivo in a mouse model of human acute myeloid leukemia. Findings from in vitro studies suggest that the anti-cancer properties of such combinations are independent of the origins of cancer cells, their mutational landscapes and P53 status. Importantly, drug-resistant cancer cells are specifically exceedingly sensitive to these combination treatments.

Described is the treatment of cancer cells with various combinations of two or more synthetic or natural compounds together that bring about catastrophic cell death. However, normal cells are spared under equivalent combination drug doses and conditions. This has been demonstrated in vitro, ex vivo, and in vivo studies. Such synergistic anti-cancer effects are unforeseen, highly efficient in killing cancer cells independent of their origins, mutational loads and P53 status, and have never been described before.

Category 3, 4, and category 5 compounds can increase the expression of human SLC7A11 among other cytoprotective genes, the overexpression of which potentiate the cytotoxicity of category 1, category 2 and category 3 compounds. It has been demonstrated that these changes are prerequisites to bolster the cytotoxicity of category 1 or category 2 molecules. Data shows that overexpression of SLC7A11 can increase the cytotoxicity of category 1 and category 2 compounds when excess of cystine and methionine are available to cancer cells. Similar biological activity can be achieved in which synthetic RNA encoding human SLC7A11 or the native SLC7A11 protein could be delivered in cancer patients to attain cancer therapeutic effects of category 1, category 2, category 3 compounds.

Expression of SLC7A11 can be increased by either independent or simultaneous activation of NFE2L2 or ATF4 by category 4 compounds. Similar properties of category 5 molecules can be found based on structure-activity analysis. Similarly, the expression of SLC7A11 can be increased by delivery of synthetic RNA encoding these proteins or by direct delivery of these proteins in cancer cells. Methods for delivery of the synthetic RNA are known in the art (e.g, Sedic, et al., Veterinary Pathology, 55:2, 341-354, (2018)). Following overexpression of NFE2L2 or ATF4, category 1, category 2, and category 3 compounds can be used either as single agent(s) or in combination to potentiate the cytotoxicity of the compounds in cancer cells.

The effect of radiation therapy can be enhanced upon administration of the described pharmaceutical compositions as cancer chemotherapeutics either before or after radiation therapy. The efficacy of such treatments can further be potentiated by prior or simultaneous strategic combinatorial use of pharmacologically accepted preparations of cystine, methionine, cysteine, N-acetyl cysteine, gamma-glutamyl cysteine, glutathione, cystathionine, vitamin C individually or in combinations. Alternatively, the side effects of radiation can be minimized with the administration of category 4 or 5 compounds when used in combination with pharmacologically accepted preparations of cystine, methionine, cysteine, N-acetyl cysteine, gamma-glutamyl cysteine, glutathione, cystathionine, vitamin C individually or in combinations.

The pharmaceutical composition can be used along with sorafenib (particularly in patients with hepatocellular carcinoma or cholangiocarcinoma or related cancer in which sorafenib is used for the treatment) among other FDA-approved cancer chemotherapeutics and/or therapeutics along with strategic administration of pharmacologically accepted preparations of cystine, methionine, cysteine, N-acetyl cysteine, gamma-glutamyl cysteine, glutathione, cystathionine, and vitamin C for treatment of cancer. The use of the pharmaceutical composition is not restricted to prior to, along with, or after the use of conventional chemotherapy.

Data shows that certain combinations of the compounds are effective against multiple cancer types independent of their tissue of origins, cumulative mutational load and P53 status. Hence, similar strategies can be applied to any malignancies in which cystine transport function of SLC7A11 is not compromised due to mutation. Data also shows that cancer cells that are deficient in methionine metabolism can be targeted with excess of cystine when used in combination with the above-mentioned pharmaceutical compositions comprising combinations of cytotoxic agents with cytotoxicity enhancing agents.

In view of the above, the category 4 or category 5 compounds can be used in combination with pharmacologically accepted preparations of cystine, cysteine, N-acetyl cysteine, gamma-glutamyl cysteine, glutathione, cystathionine, and vitamin C to mitigate cystathionine beta-synthase deficiency in a human subject.

The pharmaceutical composition can be used along with human natural or recombinant cystathionine gamma lyase [EC 4.4.1.1] for the treatment of human malignancies including melanoma. Certain category 4 compounds can dramatically increase the intracellular and extracellular cysteine and cystine levels making these enzymes to be much more effective. These enzymes can metabolize its surrogate substrate selenocystine or selenocystathionine (key compounds in category 2) to a highly cytotoxic compound compared to when they use cystine as their cognate substrate. RNA-sequencing data shows that expression of cystathionine gamma lyase is increased by a category 4 compound such as diphenyl diselenide and when used in combination, this compound increases the cytotoxicity of selenocystine in cancer cells.

### Ex Vivo Studies

### Experimental Details

Freshly resected sample of human pancreatic ductal adenocarcinoma (PDAC) was cut into thin (300 μm) slices (length: 0.5-0.7 cm, width 0.5-0.7 cm) using vibrating blade microtome. Tissue slices were cultured for 24 h for initial acclimation in the ex vivo culture condition. Subsequently, tissue slices were treated with sodium selenite (10 μM), selenocystine (20 μM), and diphenyl diselenide (5 μM) individually or with individual combination of diphenyl diselenide sodium selenite or selenocystine for 72 h. In addition, 5-FU and Gemcitabine (5 μM each, dose level is based on plasma equivalent or greater concentration of 1 week of treatment in pancreatic cancer patients) were used as standard chemotherapeutic regimen controls relevant in the clinics for pancreatic cancer patients.

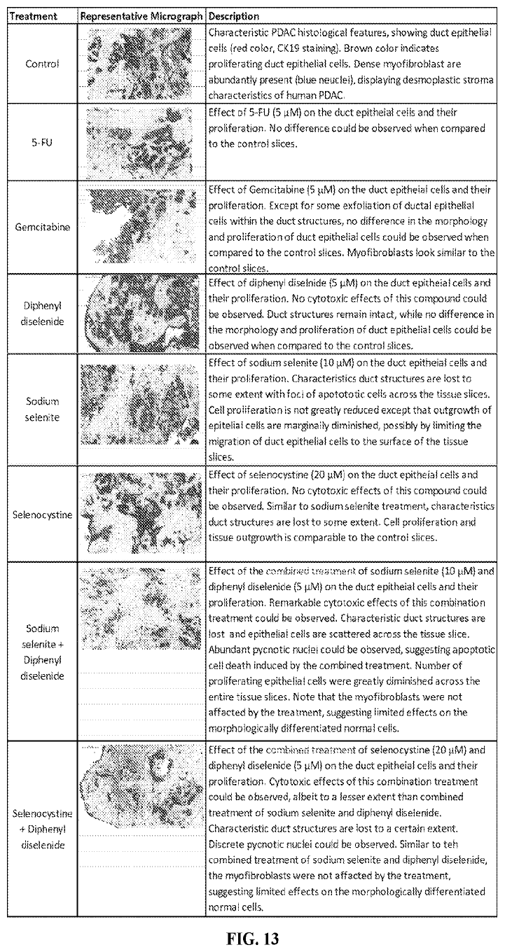

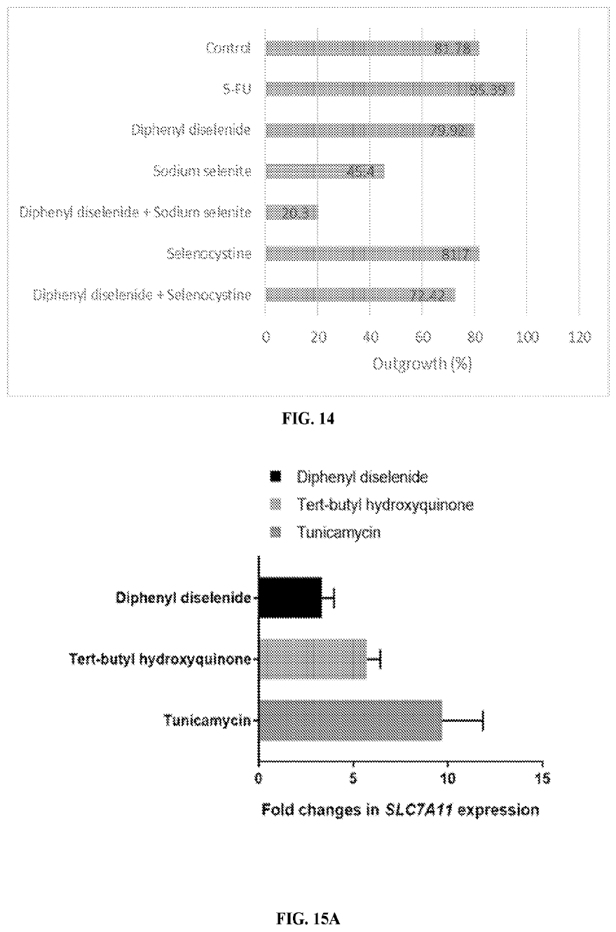

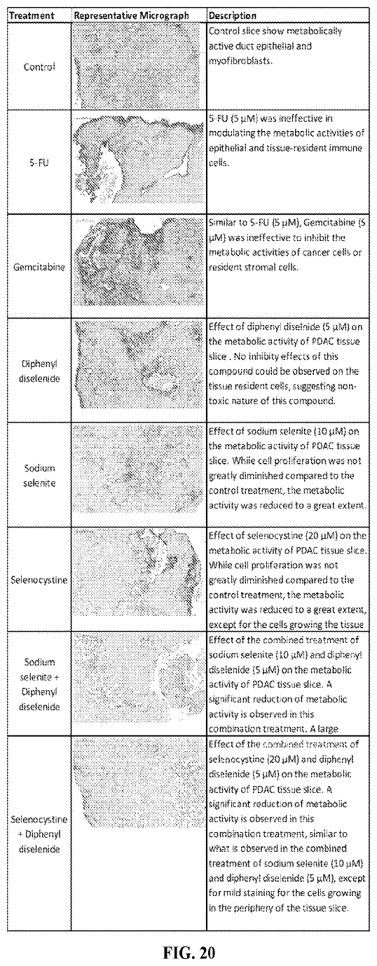

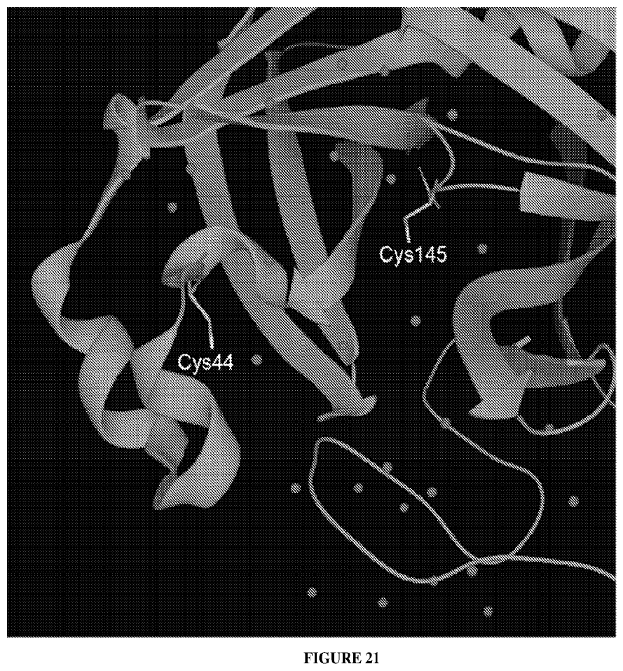

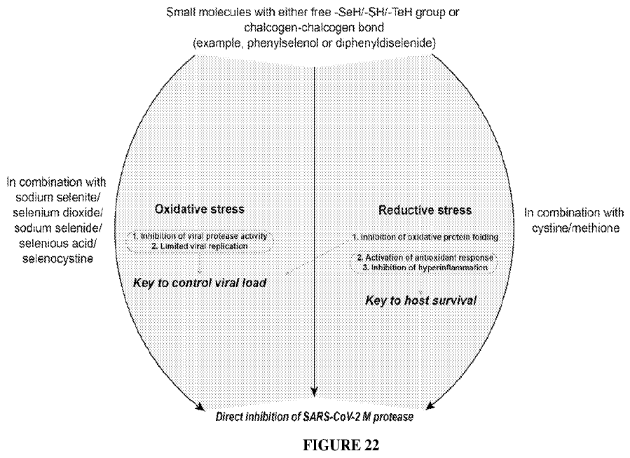

The effects of the single and combined treatments of sodium selenite, selenocystine, and diphenyl diselenide on the outgrowth and proliferation of cancer cells in human ex vivo organotypic slice culture of surgically resected primary pancreatic ductal adenocarcinoma obtained from a pancreatic cancer patient (Misra et al., Scientific Report, 2019) was tested using immunohistochemistry staining. Immunohistochemistry staining of CK19 (epithelial cell marker) and Ki67 (marker for cell proliferation) was performed following 72 hours treatments with the respective compounds either alone or in specified combinations (see FIG. 13). Outgrowth of cells in the periphery of tissue slices (a key parameter of tissue health in ex vivo culture) was measured in the different treatment groups. Outgrowth=Percentage of the tissue periphery covered by the migrating cells (see FIG. 14). Immunohistochemistry staining of pS6 (marker for metabolic activity) in these slices following 72 h treatments with the respective compounds either alone or in specified combinations (see FIG. 20).

Table 6. show suggested dose levels of sodium selenite, selenocystine, and their analogs and corresponding dose levels of diphenyl diselenide and functionally-related molecules (category 3, 4, and 5 compounds) for the treatment of human cancer based on the available data. All dose levels indicate mg/m² body surface area/day in human for a minimum of one-week treatment, without any upper limit on the duration of treatment or as recommended by physicians. Specific application indicates a loading dose range prior to the commencement of continuous infusion, as appropriate or determined by the physicians. The mentioned dose is the minimum loading dose given as a single administration in a preferred route of administration. Alternatively, multiple daily single dose of each compound can be administered using any of described method of delivery.

TABLE 6

| Representative | Minimum dose | Suggested dose | Maximum dose | Specific | | |
|---|---|---|---|---|---|---|
| Compound | levels | levels | levels | application | Specific Combinations | Ratio^{λ} |
| Sodium selenite ^{α} | 1.00 | 5.00 | 15.00 | ≥2.00 | Combined with Category 3, 4 | 0.1:250 |
| | | | | | and 5 molecules | |
| Selenocystine ^{β} | 1.00 | 9.00 | 25.00 | ≥2.00 | Combined with Category 3, 4 | 0.1:250 |
| | | | | | and 5 molecules | |
| Diphenyl diselenide ^{δ} | 1.00 | 22.00 | 100.00 | ≥2.00 | Combined with Category 1 and | 0.1:250 |
| | | | | | 2 molecules | |

^{α} Indicates Category 1 compounds and their analogs
^{β} Indicates Category 2 compounds and their analogs
^{δ} Indicates Category 3, 4 and 5 compounds and their analogs
^{λ}Range. The first number is for its respective category molecule/s and the second number is for other categories with which these molecules are combined individually)

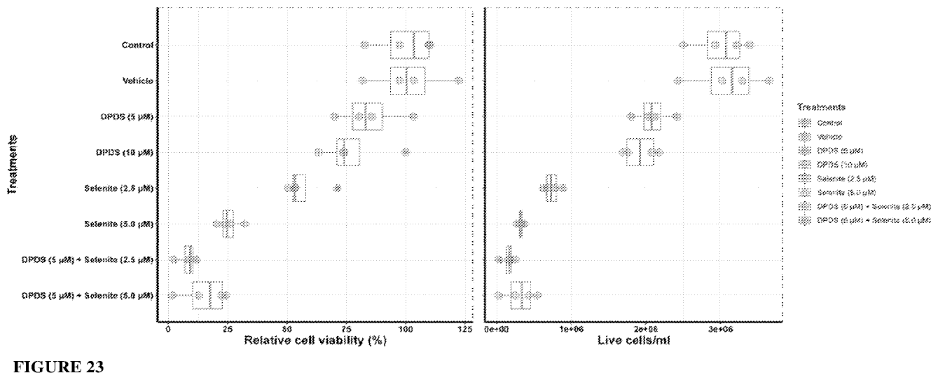

Viability of isolated splenocytes from secondary leukemic mice following single or combinatorial treatment with sodium selenite and diphenyl diselenide (DPDS) Primary mice leukemic cells bearing human MLL-AF9 oncogene were transplanted into healthy secondary mice. These transplanted mice developed aggressive leukemia within 3 weeks of transplantation and the whole spleens were collected following euthanasia. Splenocytes were isolated and cultured ex vivo in 24 well plates (1.5×10⁶ cells/0.5 ml/well) for 12 h prior to exposing with the indicated compounds for another 48 h. At the end of the exposure, cell viability was measured using an automated cell counter (Nexcelom Biosciences). As demonstrated earlier with human cancer cell lines, the combined treatment with sodium selenite and diphenyl diselenide resulted in significantly higher cell death when compared with equivalent concentrations of sodium selenite or diphenyl diselenide alone (FIG. 23 left panel). Similar results were found from live cell count data (FIG. 23 right panel). Further reduction of selenite concentration to either 1.0 μM or 0.5 μM in the combinatorial treatment yields comparable results on cell viability (<5% viable cells), suggesting it is possible to lower the concentration of sodium selenite to achieve similar effectiveness. The sample size indicates number of individual mice and the splenocytes count from each sample is averaged from 3 technical replicates.

The Combinatorial Treatment of Sodium Selenite and Diphenyl Diselenide (DPDS) at Pharmacological Concentration Selectively Kill Leukemic Cells while Normal Cells are Spared.

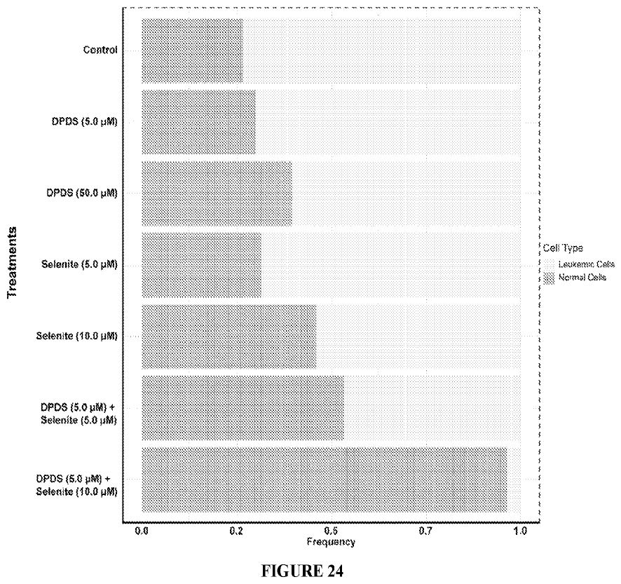

Primary mice leukemic cells (CD45.1 background) bearing human MLL-AF9 oncogene were transplanted into healthy secondary mice (CD45.2 background). Following leukemia development in these mice, spleens were collected following euthanasia. Splenocytes, comprising both normal and leukemic cells, were isolated and cultured ex vivo in 24 well plates (1.5×10⁶ cells/0.5 ml/well) for 12 h prior to exposing with the indicated compounds for another 67 h. At the end of the exposure, relative frequency of normal (CD45.1 negative) and leukemic (CD45.1 positive) cells were determined using flow cytometry. DPDS at a very high concentration (50.0 μM) had marginal inhibitory effect on the frequency of either normal or leukemic cells. Treatment with 10.0 μM sodium selenite resulted in diminished frequency of leukemic cells when compared with control cells. The combinatorial treatment of DPDS (5.0 μM) with sodium selenite (5.0 μM) elicited diminished leukemic cell frequency. Further elevated concentration of sodium selenite (10.0 μM) in combination with DPDS (5.0 μM) resulted in almost complete eradication of leukemic cell population (average frequency 3.67%) with no apparent toxic effects on the viability of normal splenocytes. These findings suggest higher sensitivity of leukemic cells towards the combinatorial treatment of sodium selenite and DPDS when compared to normal cells. See FIG. 24.

### The Combinatorial Treatment of Diphenyl Diselenide (DPDS) and Sodium Selenite is Selectively Cytotoxic to Leukemic Cells.

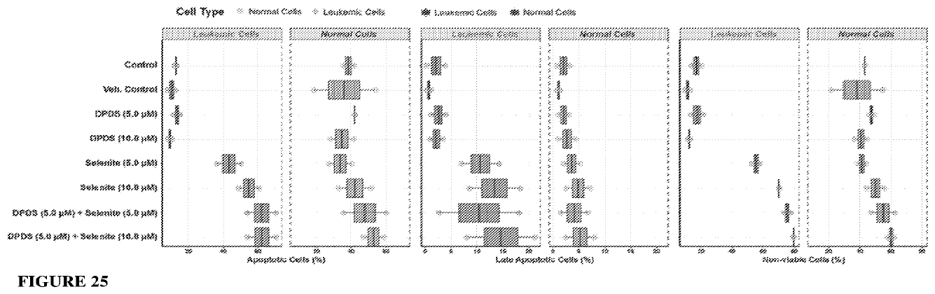

The nature of cell death at an earlier time point (48 h) is investigated to find out whether increased frequency of normal cells in the culture is due to increased leukemic cell death while normal cells are spared. Splenocytes from secondary leukemic mice were isolated and cultured ex vivo in 24 well plates for 12 h prior to the treatment with the indicated compounds either alone or in combinations for another 48 h. At the end of the exposure period, cell viability was measured using Annexin-V (apoptotic cell death) and 7-AAD (necrotic cell death). Late apoptotic cells are defined as positive for both Annexin-V and 7-AAD, while non-viable cells are designated as the total of apoptotic, necrotic, and late apoptotic cells. In relation to the control or vehicle control-treated cells, the relative increase in apoptotic, late apoptotic, and non-viable cells following the treatment with the combined treatments are higher in the leukemic cell populations when compared to normal cells. The baseline (control or vehicle control group) cell viability for normal cells was low (˜40%, related to culture methodology optimized for leukemic cells) and it did not exceed more than 60% (˜1.5-fold increase) following the combined treatment with sodium selenite and DPDS for 48 h. However, the overall leukemic cell death was ˜ 5.6-fold higher for the cells receiving combinatorial treatment of sodium selenite and DPDS when compared to the untreated cells. Notably, increasing the concentration of sodium selenite from 5.0 μM (well-tolerated pharmacological dose) to 10.0 μM didn't elicit further increase in cell death in the combined treatment. This finding suggests that DPDS is uniquely effective in killing leukemic cells when used in combination with low dose of sodium selenite that otherwise is less effective when used alone. Together, these data suggest that the combinatorial treatment of sodium selenite and DPDS is highly effective in killing murine primary leukemic cells harboring human oncogene MLL-AF9 ex vivo while normal cells are spared at equivalent concentrations. See FIG. 25.

The Combinatorial Treatment of Diphenyl Diselenide (DPDS) with Low Dose of Sodium Selenite Elicits Apoptosis in Primary Isolated Human Leukemia Cells Ex Vivo.

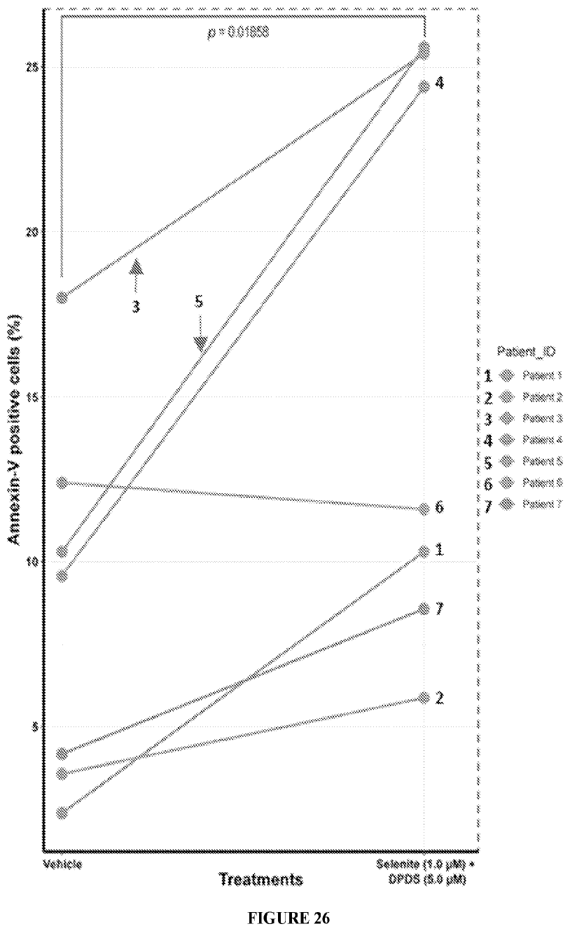

Leukemic cells were isolated using Ficoll gradient from bone marrow aspirates from seven patients (unidentified) with confirmed diagnosis of acute myeloid leukemia. These cells were cultured ex vivo in 24 well plates for 12 h prior to treatment with DPDS (5.0 μM) in combination with sodium selenite (1.0 μM). Vehicle treated cells served as control. The concentration of sodium selenite in this experiment was comparable to ‘physiological’ concentration of total selenium in the plasma of healthy humans in the US population to investigate the effectiveness of DPDS in killing patient-derived leukemic cells when combined with very low dose of sodium selenite. Findings suggest that the combined treatment with pharmacological concentration of DPDS with very low dose of sodium selenite results in increased (p-value=0.01858) apoptosis of patient-derived leukemic cells. See FIG. 26.

### Example 2

### Background

Immune response is the key mechanism of host defense against infectious agents. It has been reported now that the majority of COVID-19 related deaths are associated with older age as immune functions are negatively related with aging. Other comorbidity factors include lung and cardiovascular diseases, diabetes, hypertension, and obesity among others. It is well-known that many of these diseases are predisposing factors for dysfunction of immune system.

Three potential treatment strategies can be conceptualized to combat SARS-CoV-2 infection. The first is by neutralizing the invading virus particles either by blocking the entry of the virus using small molecules or by vaccination that mounts antiviral response by neutralizing antibodies. The second strategy involves limiting viral replication in infected patients by small molecule therapeutics. The third strategy involves minimizing the damage associated with infection specifically by limiting the hyperinflammation associated with cytokine storm leading to macrophage activation syndrome.

COVID-19 patients with severe symptoms suffer from life-threatening systemic hyperinflammation. Publicly available research data clearly demonstrate a significant correlation between infection severity and systemic oxidative stress. Recent studies have demonstrated that the viral replication and the activity of SARS-CoV-2 M protein can be diminished by redox reactive compounds.

Main protease (Mpro) of SARS-CoV-2 is a key enzyme of coronaviruses belonging to the class of cysteine proteases. It plays pivotal roles in mediating viral replication and transcription. SARS-CoV-2 Mpro has Cys-His catalytic dyad in which Cysteine 145 residue presents a ‘unique’ druggable target. This ‘critical’ cysteine residue is amenable to oxidation by redox-reactive small molecules, and thus the catalytic function of this protease can be abrogated by covalent modifications, eventually leading to inhibition of viral replication and transcription.

There is a need for redox-reactive small molecules that inhibit viral replication and transcription.

### Methods

Described are methods for the treatment of COVID-19 and similar viral diseases with fatal outcome in a human subject using different classes of small molecules either as a single agent or in all possible combinations, as outlined in the summary of the invention.

The composition can be used to both prevent and treat COVID-19 patients along with pertinent other infectious diseases characterized by cytokine storm. This invention involves limiting viral replication in infected patients by small molecule therapeutics and minimizing the damage associated with infection by limiting the hyperinflammation associated with cytokine storm leading to macrophage activation syndrome.

The concept involves use of certain small synthetic (redox-reactive organochalcogen compounds and selected selenium compounds) and endogenous small molecules either alone or in specific combinations such that systemic redox stress can be mitigated, while virus replication can be inhibited by the same combinations, albeit at higher dose. The same combinations also bolster immune system in parallel, specifically the functions of T-cells in mounting immune response, while excessive macrophage activation is blocked by these compounds at the same time. Together, this invention is poised to bring about a strategy on how COVID-19 infection can be prevented and treated by bolstering the immune system while mitigating severe oxidative damages a potential causal factor related to multiple organ failure.

Main protease (Mpro) of SARS-CoV-2 is a key enzyme of coronaviruses belonging to the class of cysteine proteases. It plays pivotal roles in mediating viral replication and transcription. SARS-CoV-2 Mpro has Cys-His catalytic dyad in which Cysteine 145 residue presents a ‘unique’ druggable target. This ‘critical’ cysteine residue is amenable to oxidation by redox-reactive small molecules, and thus the catalytic function of this protease can be abrogated by covalent modifications, eventually leading to inhibition of viral replication and transcription (see FIG. 21).

Small molecule chalcogens with functional —SeH, —TeH, or —SH group can oxidize this cysteine (C145). Among these chalcogen molecules, ones with the selenol (—SeH) functional group (e.g., diphenyl diselenide, phenylselenol, benzylselenol among pertinent others with similar chemical and biochemical properties) is predicted to have the highest inhibitory effects on SARS-CoV-2 Mpro enzyme activity. Similar inhibitory effects could be predicted on other viral cysteine-proteases. Such inhibitory activity could further be amplified by the addition of therapeutic doses of sodium selenite and selenocystine. Together, these classes of compounds can inhibit viral replication and transcription, and consequently the sequelae of complications associated with severe SARS-CoV-2 infection can be avoided.

The above-mentioned selenium molecules also exhibit certain characteristics that are unique in the context of mounting heightened immune response. These molecules change the intracellular and extracellular redox potential of cells and at systemic levels that is conducive for fighting infection by strengthening the functionalities of immune cells, specifically T-cells. This has important implications in the context of observed lymphopenia in severe COVID-19 patients. These unique properties of these compounds entail bolstering immune system to prevent SARS-CoV-2 infection.

Minimizing hyperinflammation associated with cytokine storm: Unrestricted viral replication leads to high viral titer, which, in turn, triggers hyperinflammation and associated complications such as acute respiratory distress syndrome. Immune cells of innate immune system (neutrophils, monocytes and macrophages) play critical roles in such uncontrolled inflammatory response by enhancing the production of reactive oxygen and nitrogen species. Key therapeutic strategy shall aim at interrupting the cell signaling cascades leading to hyperinflammation. The above-mentioned selenium molecules strongly augment the activity of NFE2L2, the activation of which is associated with mitigating inflammatory response and oxidative stress. Moreover, when cystine and methionine are supplied in combination, a strong antioxidant response is mounted that inhibits secondary damages associated with overactivation of innate immune cells. In fact, it has been shown that severe COVID-19 patients suffer from systemic oxidative damage with reduced levels of key antioxidant molecules in the systemic circulation. Thus, these selenium molecules and related chalcogens are uniquely poised to prevent hyperinflammation associated with severe SARS-CoV-2 infection.

One of the key elements in described is that the majority of these molecules are either small endogenous biomolecules or their structural backbone contains chalcogen molecules that are not foreign to the body. With certain exceptions, some of these molecules (specifically the compounds with —SeH functional groups) have been tested for their toxic effects in animal models. These studies have clearly demonstrated low toxicity of these compounds without any long-term side effects and the majority of drug-induced side effects were transient.

### Example 3

### Background

Acute myeloid leukemia (AML) is a group of hematological malignancies, characterized by excessive accumulation of blast cells in the bone marrows and peripheral blood^{1,2}. AML progresses rapidly and is fatal within weeks or months unless treated. Existing repertoire of anti-leukemic drugs have markedly improved the disease outcome. However, the rates of overall survival are far more sobering, highlighting the dismal outcome of relapsed leukemia following an initial response. In addition, primary and secondary drug resistance remains a pervasive issue. The complex and dynamic clonal architecture of AML are key drivers of variability in drug response and eventual emergence of secondary resistance in many patients.

Targeting oncogenic driver mutations and signaling in general have considerable therapeutic advantages, given interventions are effective and without major side-effects. However, the existence and emergence of genetically diverse clonal populations complicates therapeutic intervention3. In fact, intra- and inter-tumor clonal heterogeneity is a common signature of malignancies, including AML4. This is further compounded by drug resistance5, an ancillary cell survival mechanism designed to evade the cytopathic effects of chemotherapeutics.

Selective therapeutic targeting of adaptive metabolic vulnerabilities of leukemic blast cells and LSCs has been recently suggested as a promising approach to address clonal heterogeneity-associated therapy complexity, as is the case with AML. This approach does not solely rely on the genetic signature of leukemic cells that exhibit unparalleled divergence within and among patients. Instead, it aims at targeting unique metabolic requirements of leukemic cells and progenitor LSCs. An amenable therapeutic target in the context is metabolic pathways essential for survival of normal cells but uniquely adapted in leukemic cells such that therapeutic intervention is plausible. One such example is pathways regulating the turn-over of non-enzymatic antioxidant systems that collectively regulate the overall cellular redox homeostasis, a key physiological pathway important for survival of all living cells. Pathophysiological adaptations of some of these pathways favor selective advantages to leukemic cells for their proliferation and survival. At the same time, these adaptations render amenable therapeutic vulnerability as outlined below.

Rapidly proliferating leukemic cells generate high basal levels of reactive oxygen species (ROS). This is coupled with high levels of cystine/cysteine (CySS/CySH) and methionine uptake to establish altered homeostasis of cellular reducing equivalents that attenuate ROS-induced oxidative damage6. This survival-promoting adaptation is a signature of dysregulated redox homeostasis in leukemic cells that distinctly differs from the tightly regulated redox equilibrium of normal leukocytes and hematopoietic stem cells (HSCs)7. The disruption of cystine/cysteine and methionine acquisition pathways and their metabolic turnover in leukemic cells has been suggested as a palpable therapeutic target for intervention due to absolute essentiality of sulfur-containing amino acids in redox regulation. In association, the deprivation of these amino acids has been proposed 6. However, key limitations of such an approach are: (1) the levels of these amino acids in systemic circulation depends on dietary protein sources—all of which contain these amino acids, and (2) it is not a curative approach in the context of a fast-progressing disease.

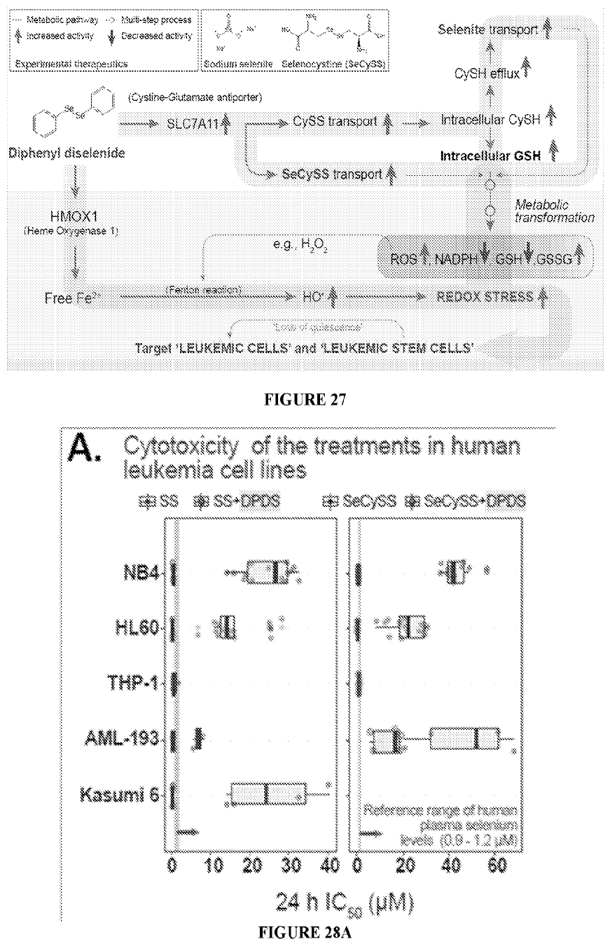

In addressing the above caveats, an alternate multi-pronged therapeutic strategy is presented (FIG. 27). It involves combinatorial treatments of diphenyl diselenide (DPDS) with either sodium selenite (will be termed as ‘selenite’ herein) or selenocystine (SeCySS). DPDS at pharmacological concentration is non-toxic and functions as an inducer of CySS transport involving increased translation of cystine-glutamate antiporter (SLC7A11). Increased SLC7A11 expression also facilitates cellular uptake of SeCySS due to structural similarity between CySS and SeCySS. DPDS-induced augmented CySS uptake enhances glutathione (GSH) biosynthesis and induces CySH efflux. Effluxed CySH reduces selenite to selenide intermediates. These processes collectively aid in efficient delivery of the intermediate pro-drug entities into the target cells. Mechanistically, these small molecule thiols (GSH and CySH) facilitate the metabolic transformation of selenite and SeCySS—pathways in which ROS are generated as byproducts in a process reminiscent of bioreductive activation. Persistent loss of intracellular GSH, nicotinamide adenine dinucleotide phosphate (NADPH) among other reducing equivalents in association further reinforces oxidative insults to cells.

These metabolic pathways cooperatively lead to exacerbation of oxidative stress and culminate into unprecedented cancer cell death in which GSH and CySH paradoxically function as cytotoxicity-inducing endogenous antioxidants. Given that leukemic cells are highly sensitive to redox stress, outlined combinatorial treatments offer unique therapeutic potential without overt reliance on specific genetic mutations, as is found in majority of AML cases. Such uniqueness also offers their potential therapeutic applications in other cancer types.

Contemporary studies provide multiple lines of experimental evidence in strong support of dysregulated redox homeostasis in leukemic blast cells and LSCs. Also, findings are presented with how such pathophysiological adaptations are amenable therapeutic targets of redox stress-inducing chemotherapeutics for the treatment of leukemia. Altered antioxidant status is a distinct feature of leukemic cells of AML origin. Intracellular ROS levels have been shown to be significantly higher in leukemic blast cells isolated from AML patients diagnosed with AML8,9, acute lymphoblastic leukemia10, and chronic myeloid leukemia11 when compared to normal leukocytes. As with intracellular ROS levels, extracellular ROS levels are also higher in majority of AML patients12. Earlier studies have demonstrated increased expression of oxidative stress responsive genes in AML patient samples, suggesting prevalence of chronic redox stress in leukemic cells13,14. A higher ROS level in leukemic blasts is a better prognostic marker of survival following anti-leukemic chemotherapy15. Exacerbation of ROS generation is a common feature of many of these cytotoxic anti-leukemic drugs (e.g., daunorubicin, idarubicin, cytarabine, and etoposide). This suggests plausible therapeutic benefits of amplifying redox stress in leukemic cells. Existing therapeutic modalities are reasonably effective in targeting leukemic blast cells, but not the LSCs that are implicated in the disease relapse. However, LSCs are specifically susceptible to redox stress compared to normal hematopoietic stem cells (HSCs)16. Unlike HSCs, dependence on oxidative phosphorylation for energy production by LSCs17 makes these further susceptible to redox-active agents, specifically those that target mitochondrial respiration chain. Together, redox stress-inducing therapeutics offer the unique possibilities to collaterally target both bulk leukemic blast cells and LSCs in which preexisting amplification of redox stress in these cells aids in therapeutic targeting.

### Material and Methods:

Slc7a11 knock-out mice: Given that Slc7a11 has been implicated in the observed therapeutic effects, it would be necessary to demonstrate (as a proof-of-concept) that leukemic cells lacking Slc7a11 gene do not respond to the outlined therapeutic intervention. These mice will also aid in our understanding of the role of this transporter in drug resistance.

### Results and Discussion

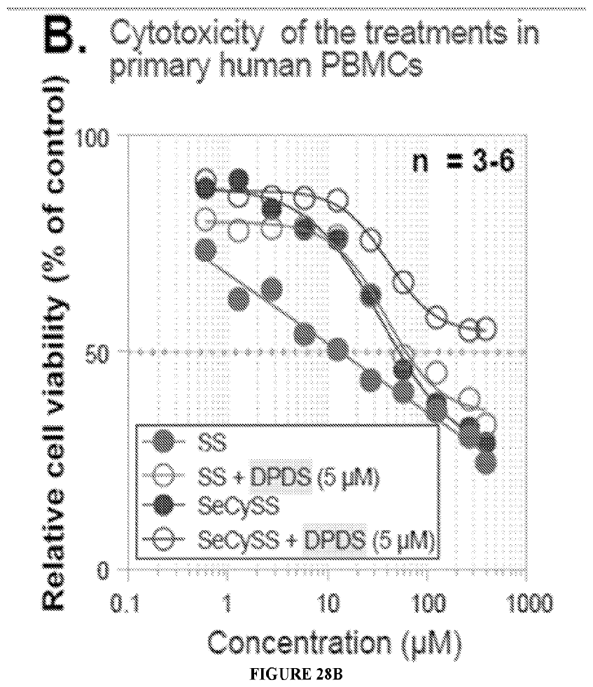

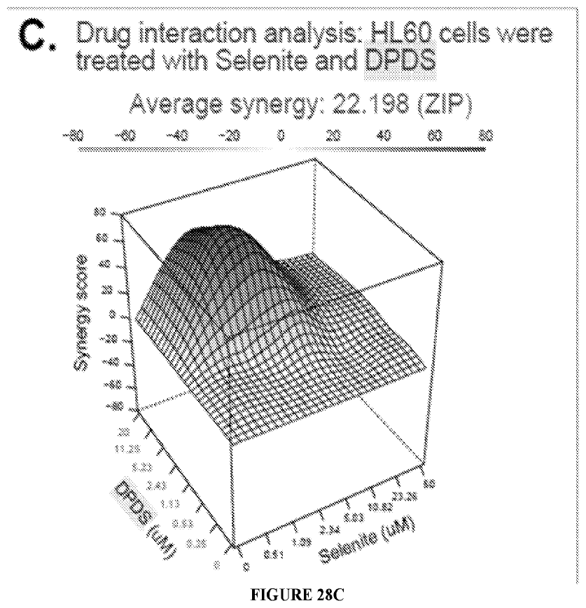

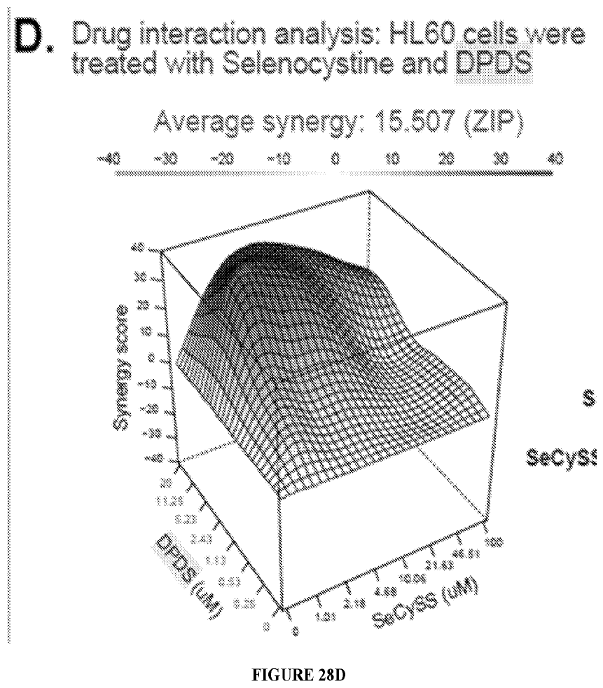

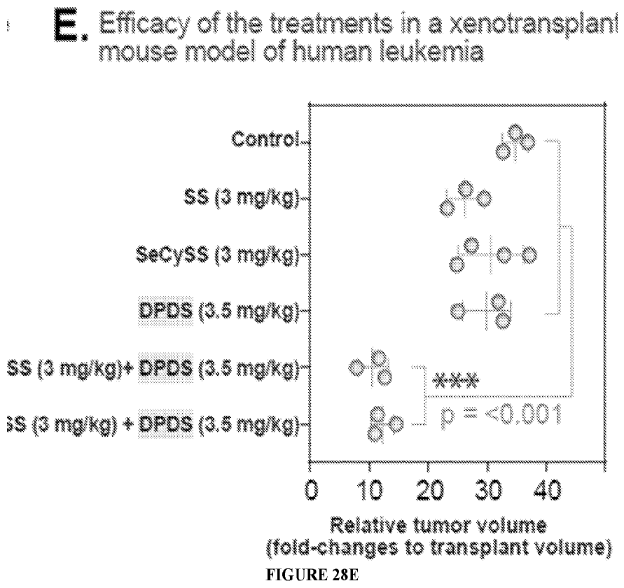

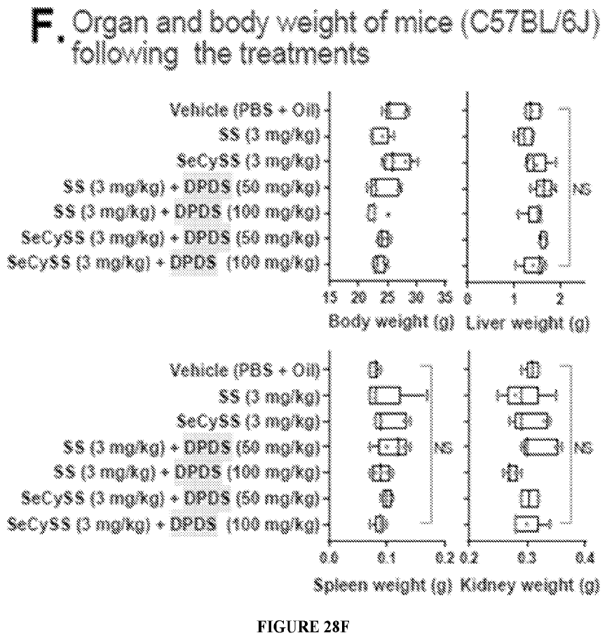

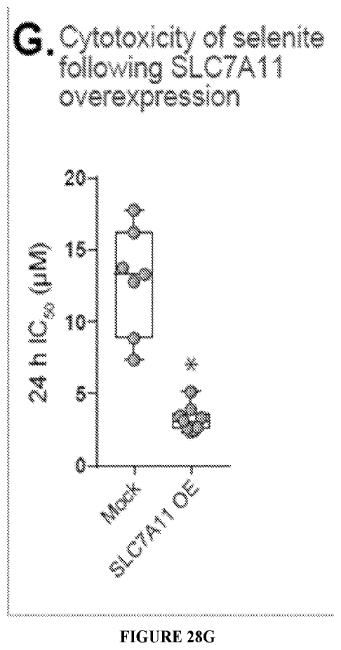

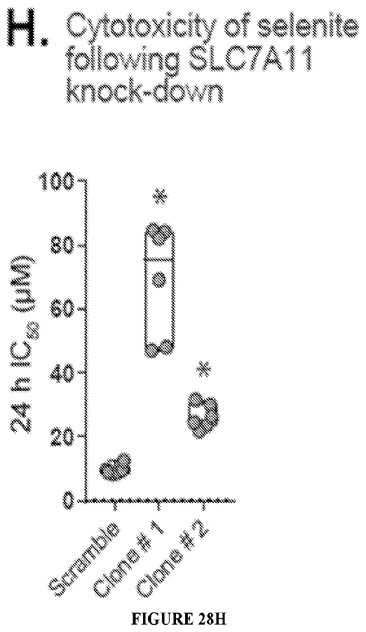

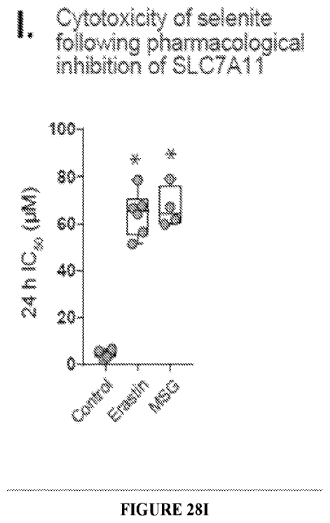

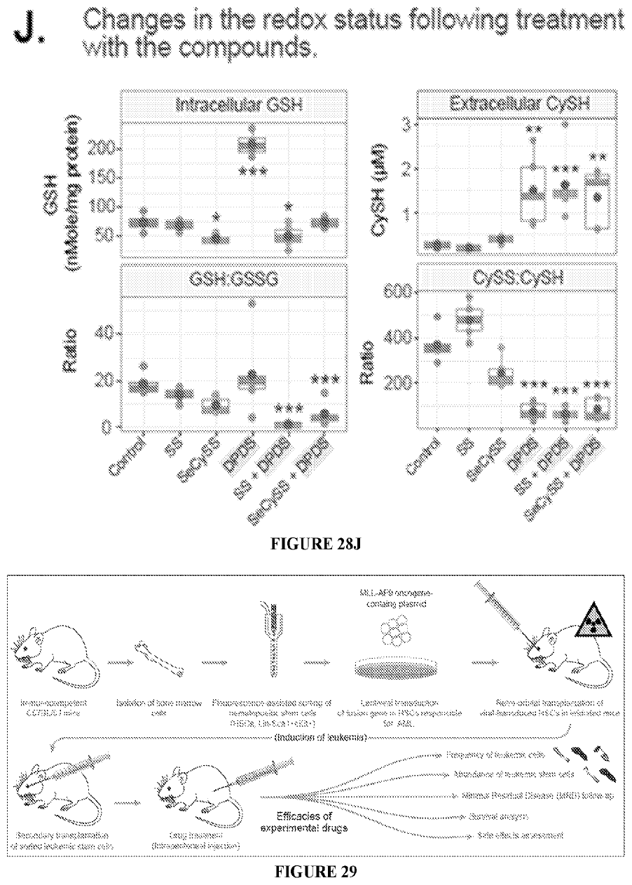

Results demonstrate that the redox anomaly of leukemic cells can be selectively targeted when DPDS is individually combined with selenite and selenocystine (FIG. 28). In conjunction, findings from relevant studies have been cited to highlight the safety of selenite and selenocystine in humans when administered at high dose levels. Aforementioned combinatorial treatments inflict unprecedented leukemic cells death (FIG. 28A), while normal human peripheral blood mononuclear cells (PBMCs) (FIG. 28B) and primary hepatocytes (data not shown) are not affected at the equivalent dose levels, suggesting limited collateral damage to normal cells. In vitro studies demonstrate that when combined with DPDS (cytotoxicity-modifying agent), the required dose levels of selenite and selenocystine (pro-drug entities) to kill 50% leukemic cells in 24 h (24 h IC50; range: nanomolar to low micromolar levels) are well within the physiological levels of plasma total selenium (0.9-1.2 μM). Among different cancer cell types tested, leukemic cells are the most sensitive to these combined treatments. Drug interaction analysis is suggestive of unparalleled synergistic effects of the combinatorial treatments. Combined treatment of DPDS with sodium selenite in an AML cell line yields an average synergy score of 22.2, suggesting exemplary efficacy of this combined treatment (FIG. 28C, FIG. 6A). The combinatorial treatment of DPDS and selenocystine (FIG. 28D) was similarly effective. In comparison, when venetoclax (currently considered to be one of the best next-generation anti-leukemic drug) is combined with eltanexor, the average synergy score has been reported to be 7.091 in another AML cell line18. Maximum-tolerated dose levels of intravenously administered selenite in human is 10.2 mg/m2 that gives rise to peak plasma total selenium concentrations ranging between 15.0-23.0 μM19. No dose-limiting toxicity was found at this dose level. Similarly, it has been reported that when selenocystine was orally administered in humans (dose levels varied between 50-200 mg/day for 49 days), no signs of drug-induced toxic effects were reported20. Plasma concentration of selenocystine was not measured in this study. A conservative estimation with the assumption that 15% of daily average ingested dose of selenocystine (set at 100 mg) was bioavailable, this will give rise to a maximum plasma concentration of selenocystine to 17.8 μM (average blood volume=5 L). These findings indicate that selenocystine and selenite are well-tolerated in human when administered at pharmacologically-relevant dose levels. Collectively, the above findings indicate the plausible existence of a therapeutic window for selenocystine and selenite for which suggested therapeutic plasma concentration lies in the range between 5-10 PM. Findings from my unpublished study demonstrate that the combined treatments are efficient in inhibiting the progression of leukemia in vivo in an aggressive secondary xenotransplant mouse model of human leukemia (FIG. 28E). The required dose levels of the experimental drugs to achieve this response did not elicit any side effects, both at organs level (organ weight and histological evaluation of major organs) (FIG. 28F) and as assessed by behavioral abnormalities. These results corroborate with the leukemic cell killing efficacy from in vitro studies. The administered dose levels of selenite and selenocystine in this study were set well within the therapeutic window in human, following the FDA guidelines on human-to-animal dose conversion factor. One of the mechanisms of action involves DPDS-induced overexpression of SLC7A11. This has been verified using plasmid-based overexpression of SLC7A11 that elicits augmented sensitivity of cancer cells towards selenite (FIG. 28G) and selenocystine. Short interfering-RNA-mediated (FIG. 28H) and pharmacological inhibition of SLC7A11 (FIG. 28I) reverses this effect. It has also been demonstrated that the combined treatments significantly alter the redox potential of intracellular GSH and GSSG couples to an oxidizing state, thereby further exacerbating oxidative insults to the leukemic cells (FIG. 28J).

In summary, the combined treatments of DPDS with either selenite or selenocystine at pharmacological dose levels demonstrate unique therapeutic prospects in targeting leukemia. The inventor has also identified several small molecules that exhibit similar biochemical properties as with DPDS, albeit with different potency in inducing cell death when combined with selenite and selenocystine. These are listed in the provisional patent application.

Outlined findings from in vitro and in vivo studies involving a highly aggressive mice model of human leukemia are highly reassuring. However, a major weakness is that the utilized xenotransplant murine leukemia model fails to recapitulate key biological aspects of leukemia in which significant numbers of leukemic cells originate and reside in the bone marrow and metastasize to multiple organs. Also, the use of immunocompromised mice eliminates the potential roles of immune cells, if any there. This structured proposal will exactly address this with a defined objective of gathering preclinical in vivo efficacy data prior to initiating large animal studies and a subsequent IND application.

Mouse model of AML: We will utilize an established and ongoing mouse model (C57BL/6J background) of AML21 Leukemia, being a stem cell disease, can be easily propagated by serial transplantation of leukemia stem cells. As reported earlier, these mice are generated by transplanting bone marrow-derived hematopoietic stem cells (HSCs, Lin-Sca1+cKit+) that are transduced with a lentivirus harboring the human fusion oncogene MLL-AF9 responsible for leukemia development (FIG. 29). Stable genomic integration of the oncogene in HSCs converts these to LSCs with loss of Sca1 expression. When engrafted (6-8 weeks), LSCs repopulate in the bone marrow, proliferate, and are used for secondary transplantation following cell sorting. The secondary recipient mice develop aggressive disease and will be utilized for in vivo efficacy assessment. To assess the treatment efficacy and bone marrow toxicity at the same time, LSCs generated from B6.SJL congenic mice with CD45.1 allele background will be retro-orbitally transplanted into healthy CD45.2+ recipient mice, allowing us to distinguish donor LSCs (Lin-Sca1-cKit+CD45.1+) from recipient HSCs (Lin-Sca1+cKit+CD45.2+). Secondary transplanted AML mice will be treated with the experimental drugs either as single agents or in combinations.

Experimental design: Like any other cytotoxic drugs, over-dosing of selenite and selenocystine can be highly toxic. Moreover, in vitro studies demonstrate strong synergy when these pro-drugs are combined with DPDS. Performed cytotoxicity studies employing selected normal cells do not warrant unintended side-effects in vivo. Hence, finding the right therapeutic window will be the single most important factor that may dictate the outcome of the therapeutic intervention—whether it is a failure or success. The proposed study will be divided into two phases to derisk the above uncertainty. Dose escalation phase-I study involving 15 different treatments (see below) will aim at finding the toxicity, maximum tolerated dose, and preliminary efficacy in secondary transplanted murine model of AML in vivo (n=6 per treatment group; statistical power=0.84 with an effect size of 0.5 and a predefined Q error probability of 0.05). Based on the findings from an earlier in vivo study, the dose of DPDS will be set at 7.0 mg/kg BW (non-toxic) and 3 dose levels of selenite and selenocystine (0.1, 1.5, and 3.0 mg/kg BW) are selected for single and combined treatments. Findings from the phase-I study will be evaluated and a single dose of each of the experimental drugs will be selected for the phase-II study. This study will have 7 treatment groups comprising 11 animals/per treatment (yielding a statistical power of 0.82 with an effect size of 0.45 and a predefined Q error probability of 0.05). Standard therapeutic regimen as a treatment arm has also been considered. However, literature is replete with such studies and findings on efficacy will be compared with, as applicable. An ethical application to IACUC for the study is under review.

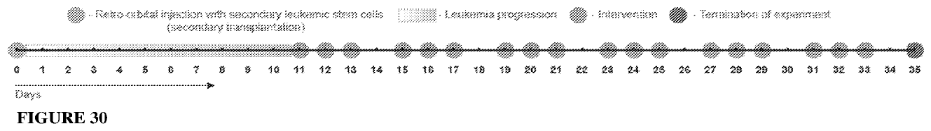

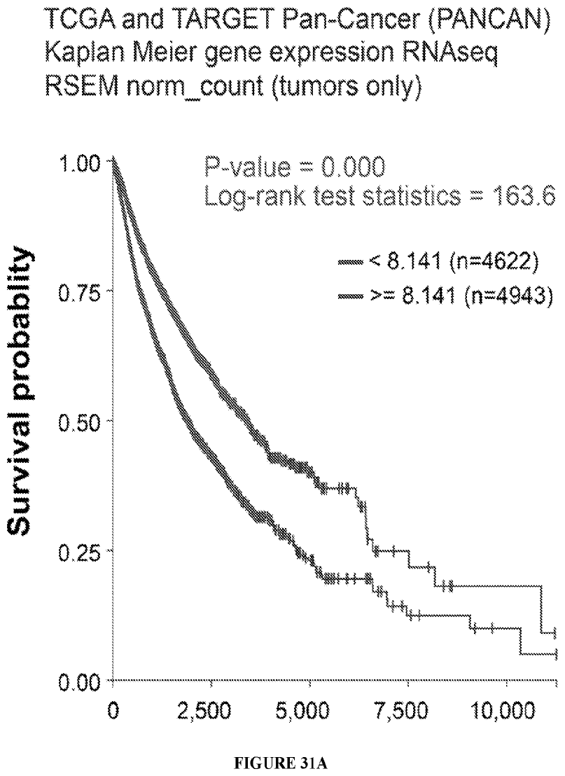

Treatment schedule: Mimicking the aggressive treatment regimen in the clinical settings and comprehensive dose-response analysis of the earlier animal study, secondary transplanted leukemic mice (10 days post transplantation) will be treated with the pro-drugs either alone or in the specified combinations for 6 cycles (FIG. 30). Details of the treatments in phase-I study. Single dose of sodium selenite and selenocystine in phase-II study will be selected from phase I study: 1. Vehicles (PBS and 2-Hydroxypropyl)-o-cyclodextrin dissolved in PBS); 2. Sodium selenite (3 doses; 0.1, 1.5, and 3 mg/kg BW dissolved in PBS); 3. Selenocystine (3 doses; 0.1, 1.5, and 3 mg/kg BW dissolved in PBS); 4. DPDS (7 mg/kg BW, prepared in 2-Hydroxypropyl)-o-cyclodextrin solution); 5. DPDS+sodium selenite (3 combinations, 7 mg DPDS/kg BW+0.1/1.5/3.0 mg sodium selenite/kg BW); 6. DPDS+selenocystine (3 combinations, 7 mg DPDS/kg BW+0.1/1.5/3.0 mg selenocystine/kg BW). Total: 15 treatments. For phase-II study, there will be a total of 7 treatments. Each treatment cycle refers to single bolus dose (mg/kg body weight) daily for 3 consecutive days followed by no treatment on day 4. Treatment gap is implemented as washout period to avoid toxicity. In case of unintended toxicity appearing at the highest dose level in the phase-I trial, additional 6 mice will be treated with an intermediate dose level residing between the dose level inflicting toxicity and nearest non-toxic doses. All the treated animals will be closely monitored and any signs of distress (lethargy, loss of grip, diarrhea among others) in the treated animals will be used as indicators of toxic effects. The study will be terminated one day post the last treatment.

End-point analyses: In the phase-I study, the endpoint analyses will comprise: (1) treatment-induced side effects during treatment (behavioral distress) and at necropsy (complete blood count, gross organ morphology, organ weight, abundance and frequency of HSCs in the bone marrow as measure of bone marrow suppression, histological assessment of liver, kidney, and spleen, and Slc7a11 expression in these tissues); (2) the abundance and frequency of bulk leukemic cells in the peripheral blood, bone marrow, and spleen; (3) survival; (4) plasma total selenium levels and redox status (measurement of small molecule thiols by LC-MS/MS); and (5) cytosolic and mitochondrial ROS levels in leukemic cells. In phase-II study, all the above end-points will be assessed along with assays for stemness and clonal expansion capacity of LSCs in ex vivo studies and minimal residual disease (serial transplantation) in a sub-set of surviving animals, if found so. Majority of the outlined analytical methods are routinely performed in our laboratory. Analyses requiring specialized instrumentation will be performed as paid research services (see details in the budget section).

Additional Experiments: Promising findings from above studies would lead to further experiments on a patient-derived xenograft (PDX) mouse model of leukemia. Patient-derived AML cells are xenografted into the femur of NOD-scid IL2Rgammanull (NSG) mice that develop full blown disease. To test the efficacy of the treatments in these leukemic mice, we will adopt a similar approach as outlined above in the phase-II study. In addition, Dr. Hong's lab has developed a unique humanized mouse model of leukemia in which a human immune system is reconstituted upon transplantation of G-CSF mobilized peripheral blood mononuclear cells. Primary transplanted leukemic mice with humanized immune system will be utilized for efficacy testing, if the findings from the first PDX mice model are satisfactory i.e., the combination treatments exert discernible anti-leukemic effects.

### REFERENCES

1. Estey E. Acute Myeloid Leukemia—Many Diseases, Many Treatments. N Engl J Med. 2016; 375(21):2094-2095.
2. De Kouchkovsky I, Abdul-Hay M. ‘Acute myeloid leukemia: a comprehensive review and 2016 update’. Blood Cancer J. 2016; 6(7):e441.
3. Vosberg S, Greif P A. Clonal evolution of acute myeloid leukemia from diagnosis to relapse. Genes Chromosomes Cancer. 2019; 58(12):839-849.
4. Grove C S, Vassiliou G S. Acute myeloid leukaemia: a paradigm for the clonal evolution of cancer? Dis Model Mech. 2014; 7(8):941-951.
5. Filipits M, Stranzl T, Pohl G, et al. Drug resistance factors in acute myeloid leukemia: a comparative analysis. Leukemia. 2000; 14(1):68-76.
6. Jones C L, Stevens B M, D'Alessandro A, et al. Cysteine depletion targets leukemia stem cells through inhibition of electron transport complex II. Blood. 2019; 134(4):389-394.
7. Testa U, Labbaye C, Castelli G, Pelosi E. Oxidative stress and hypoxia in normal and leukemic stem cells. Exp Hematol. 2016; 44(7):540-560.
8. Sallmyr A, Fan J, Rassool F V. Genomic instability in myeloid malignancies: increased reactive oxygen species (ROS), DNA double strand breaks (DSBs) and error-prone repair. Cancer Lett. 2008; 270(1):1-9.
9. Hole P S, Pearn L, Tonks A J, et al. Ras-induced reactive oxygen species promote growth factor-independent proliferation in human CD34+ hematopoietic progenitor cells. Blood. 2010; 115(6):1238-1246.
10. Battisti V, Maders L D, Bagatini M D, et al. Measurement of oxidative stress and antioxidant status in acute lymphoblastic leukemia patients. Clin Biochem. 2008; 41(7-8):511-518.
11. Ciarcia R, d'Angelo D, Pacilio C, et al. Dysregulated calcium homeostasis and oxidative stress in chronic myeloid leukemia (CML) cells. J Cell Physiol. 2010; 224(2):443-453.
12. Hole P S, Zabkiewicz J, Munje C, et al. Overproduction of NOX-derived ROS in AML promotes proliferation and is associated with defective oxidative stress signaling. Blood. 2013; 122(19):3322-3330.
13. Hole P S, Darley R L, Tonks A. Do reactive oxygen species play a role in myeloid leukemias? Blood. 2011; 117(22):5816-5826.
14. Bowen D, Wang L, Frew M, Kerr R, Groves M. Antioxidant enzyme expression in myelodysplastic and acute myeloid leukemia bone marrow: further evidence of a pathogenetic role for oxidative stress? Haematologica. 2003; 88(9):1070-1072.
15. Mondet J, Presti C L, Garrel C, et al. Adult patients with de novo acute myeloid leukemia show a functional deregulation of redox balance at diagnosis which is correlated with molecular subtypes and overall survival. Haematologica. 2019; 104(9):e393-e397.
16. Zhang H, Fang H, Wang K. Reactive oxygen species in eradicating acute myeloid leukemic stem cells. Stem cell investigation. 2014; 1:13-13.
17. Lagadinou E D, Sach A, Callahan K, et al. BCL-2 inhibition targets oxidative phosphorylation and selectively eradicates quiescent human leukemia stem cells. Cell stem cell. 2013; 12(3):329-341.
18. Fischer M A, Friedlander S Y, Arrate M P, et al. Venetoclax response is enhanced by selective inhibitor of nuclear export compounds in hematologic malignancies. Blood advances. 2020; 4(3):586-598.
19. Brodin O, Eksborg S, Wallenberg M, et al. Pharmacokinetics and Toxicity of Sodium Selenite in the Treatment of Patients with Carcinoma in a Phase I Clinical Trial: The SECAR Study. Nutrients. 2015; 7(6):4978-4994.
20. Finch E R, Kudva A K, Quickel M D, et al. Chemopreventive Effects of Dietary Eicosapentaenoic Acid Supplementation in Experimental Myeloid Leukemia. Cancer Prev Res (Phila). 2015; 8(10):989-999.

### Example 4: Microenvironment Redox Control by SLC7A11 is a Druggable Target in Cancer

### Abstract

Redox anomaly is a common metabolic signature of malignant cells.

Hyperproliferation-associated increased nutrient turnover and oncogenic signaling converge to establish a condition favorable for survival of cancer cells under adaptive redox control. Cystine-glutamate antiporter (SLC7A11) plays a key role in this process by supplying cystine for glutathione biosynthesis. In this study, we explored the possibility of targeting SLC7A11-mediated microenvironment redox control of cancer cells. It is long known that intermolecular interaction between certain redox-active selenium compounds with thiols generate reactive oxygen species (ROS) which are highly cytotoxic beyond threshold levels. We tested the hypothesis of whether the induction of SLC7A11 expression would result in augmented GSH biosynthesis, cysteine efflux and enhance the cytotoxicity of two redox-active selenium compounds—sodium selenite and selenocystine. Using molecular biology and pharmacological tools, we demonstrate that the induction of SLC7A11 expression is associated with modulation of intracellular and extracellular thiols redox potential and increased cytotoxicity to sodium selenite and selenocystine. Together, these results demonstrate that the strategic induction of SLC7A11 by small molecules or nucleic acid delivery technology has unique therapeutic potential in human malignancies when used in combination with therapeutic dosage of sodium selenite and selenocystine.

### Introduction

Redox-active selenium compounds are promising candidates for redox-directed cancer chemotherapy¹. Sodium selenite and selenocystine are two such candidate small molecules that have been demonstrated to exert notable anti-cancer efficacy in preclinical studies^{2,3}. These molecules either directly or upon metabolic transformation react with endogenous thiols⁴ and generate ROSs. Resulting imbalance in thiols homeostasis and oxidative damages along with perturbation of other key metabolic pathways are implicated in cell death².

High proliferative activity in cancer cells is associated with increased nutrient turnover and metabolic activity⁶. Maintenance of redox homeostasis is essential to support the metabolic requirements of proliferating cells, as majority of these cellular processes are intricately entwined with redox equilibrium⁷. This is mediated by elevated influx of nutrients that support redox equilibrium⁸. Sulfur-containing amino acids, cystine and methionine, play major roles in this process by supporting glutathione biosynthesis and methylation process among pertinent others⁹. In association, it has been demonstrated that a cystine antiporter, encoded by SLC7A11, is overexpressed in majority of cancer tissues when compared to their normal counterparts. While increased cystine import by this antiporter system is well known, there is little known about whether cystine acquisition-induced modulation of the redox potential of the local microenvironment of cancer cells is therapeutically tractable.

Regulation of thiol homeostasis by SLC7A11 has direct implications on the cytotoxic effects of the above-mentioned redox-active selenium compounds. The reactivity of these small molecule selenium compounds with thiols presents a unique opportunity to target cancer cells, the majority of which exhibit an adaptive state of redox homeostasis to counteract oxidative metabolism¹⁰. In accordance, it has long been known that experimental modulation of extracellular thiols redox potential increases selenium delivery¹¹ and augment the cytotoxicity of sodium selenite. The potential involvement of SLC7A11 in this process has been speculated in cancer cells¹². This antiporter is also of specific interest from the perspective of using selenocystine as cancer chemotherapeutic application due to close structural similarities between its cognate substrate cystine with selenocystine. Although cancer cells in general overexpress SLC7A11, it is not known whether this uniqueness prevails in the context of intra- and inter-tumor clonal heterogeneity. This suggests that the induction of SLC7A11 expression would be a plausible way to overcome such complexity that may have pan-cancer therapeutic implications. Chemical biology or nucleic acid-based approach presents an amenable solution for this by targeting the SLC7A11-regulatory transcription factors NFE2L2 and ATF4¹³—the induction of which results in increased expression of this transport protein. Such targeted approach may have superior cancer chemotherapeutic applications compared to using sodium selenite and selenocystine as single agents.

In addressing the above, this forward drug discovery study was designed to elucidate whether chemical biology- and nucleic acid-based approach of induction of SLC7A11 and its regulatory transcription factors would synergize the cytotoxicity of these small molecule selenium compounds to cancer cells upon modulating the redox potential of intracellular and extracellular milieu of cancer cells using in vitro models of human cancer.

### Results

### SLC7A11 is Important for Maintaining the Redox Buffer of Extracellular Milieu in Cancer Cells

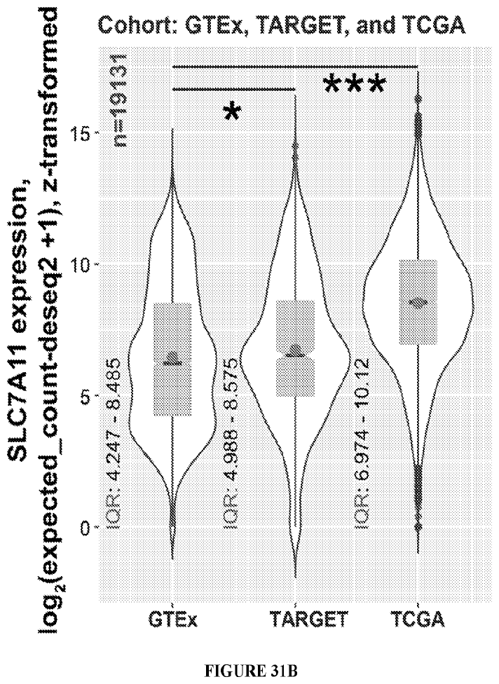

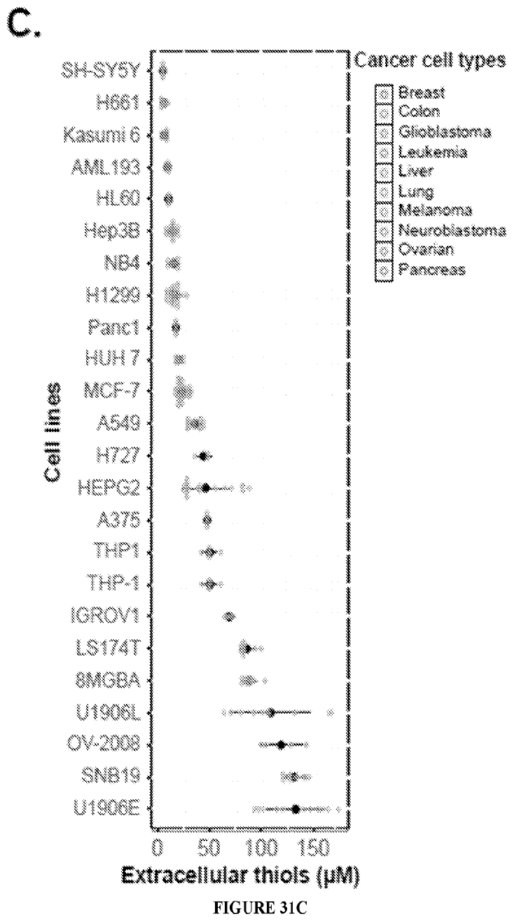

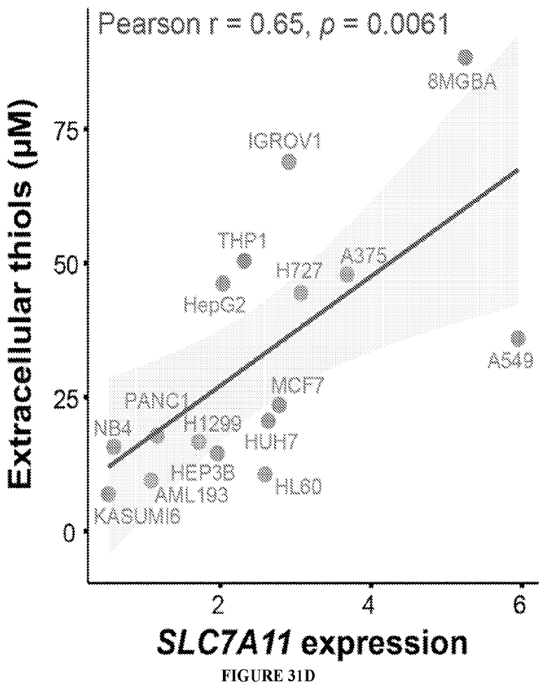

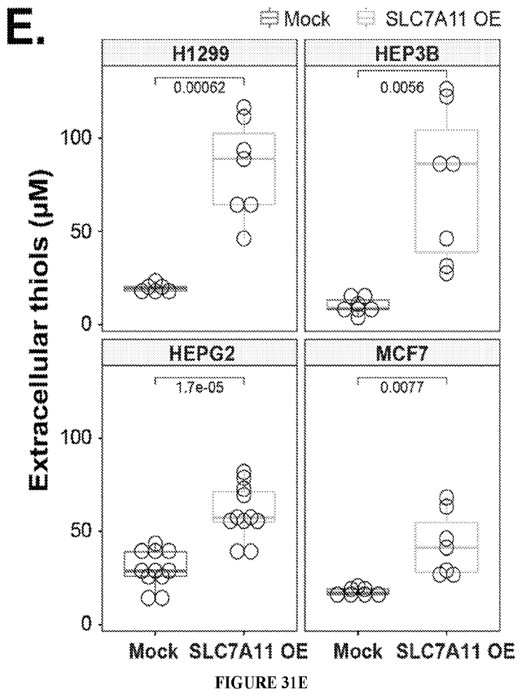

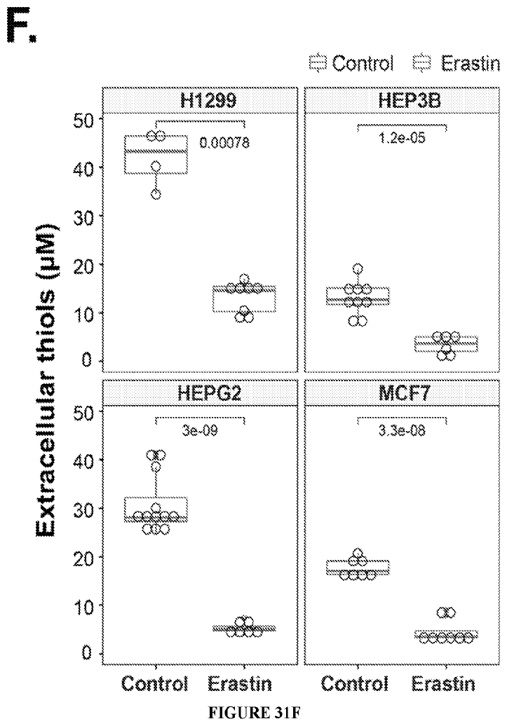

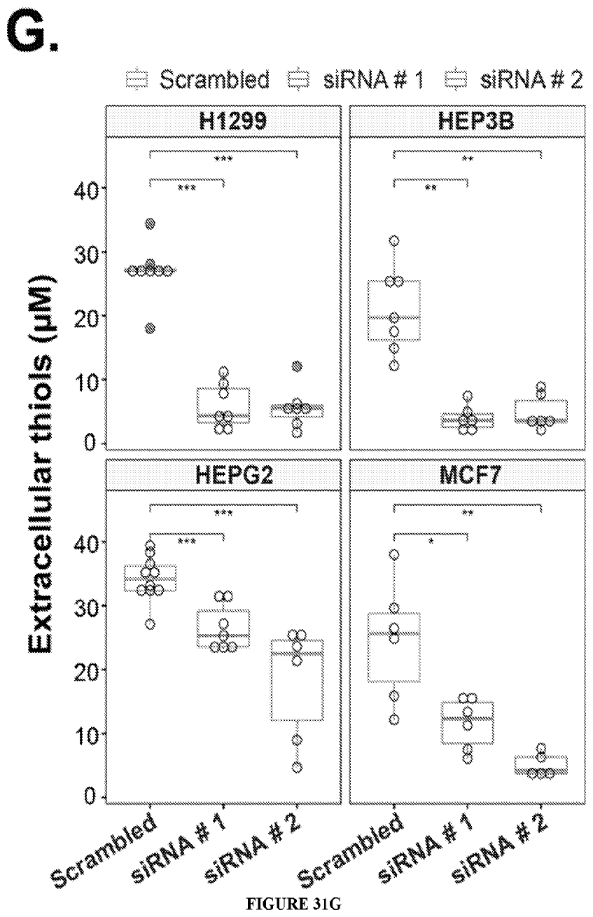

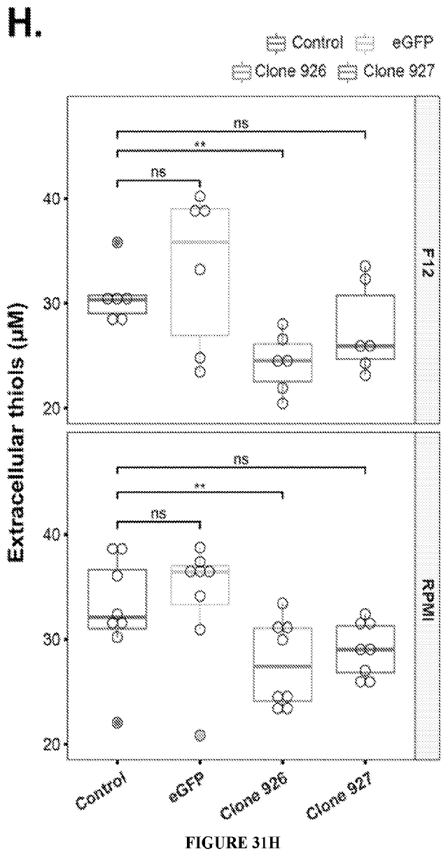

High cystine-glutamate exchanger (SLC7A11) mRNA expression is a poor prognostic marker in cancer patients (FIG. 31A). Pan-cancer gene expression analysis demonstrates SLC7A11 is overexpressed in malignancies when compared to normal tissues (FIG. 31B). SLC7A11 plays a key role in cellular cystine acquisition (7358676) which is critical for cancer cells to maintain their redox balance. Cystine import, its subsequent reduction to cysteine, and the efflux of the later are key determinants for maintaining the redox potential of the interstitial fluid and systemic circulation¹⁴. We initially measured the extracellular reduced thiols levels in 23 different cancer cell lines (FIG. 31C). High variations in extracellular total thiols levels among the cell lines was found to be associated with the levels of SLC7A11 mRNA expression (FIG. 31D). Next, we performed a series of experiments to test the factual relationship between SLC7A11 expression and the thiols redox potential of extracellular milieu. Plasmid-based overexpression of SLC7A11 resulted in increased levels of extracellular reduced thiols in all the tested cell lines (FIG. 31E). Opposite effect was observed when these cells were treated with Erastin, a specific inhibitor SLC7A11 activity (FIG. 31F). Corroborating to this observation, siRNA-based transient knock-down of SLC7A11 resulted in diminished levels of reduced thiols in the local milieu (FIG. 31G). We further validated the findings using two different clones of A549 cells that are stably transfected with SLC7A11 shRNAs. A significant reduction of extracellular reduced thiols levels was found only in the clone #926 that exhibited efficient knock-down of SLC7A11 (FIG. 31H), as reported in an earlier study¹⁵. Together, these findings suggest that SLC7A11 plays a key role in cellular cystine import that determines the redox potential of extracellular milieu.

### NFE2L2 Inducers Modulate Extracellular Redox Potential

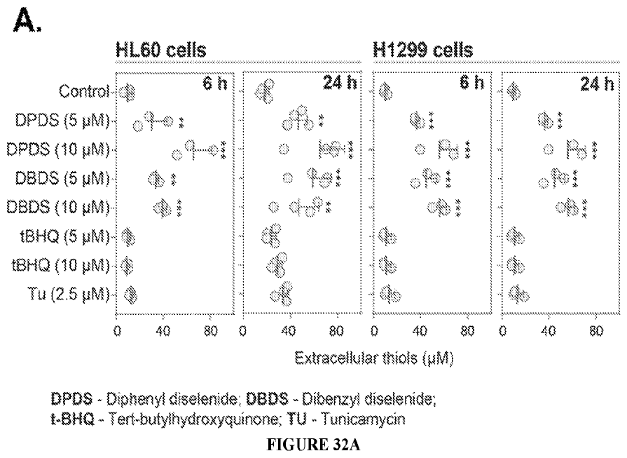

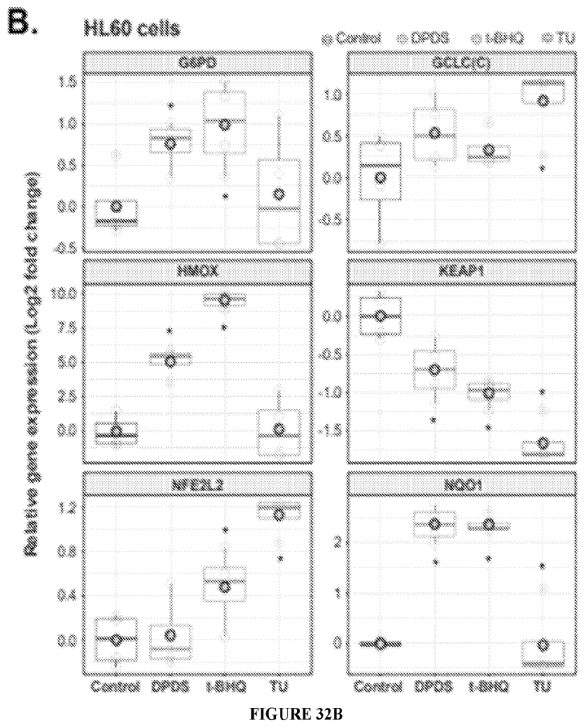

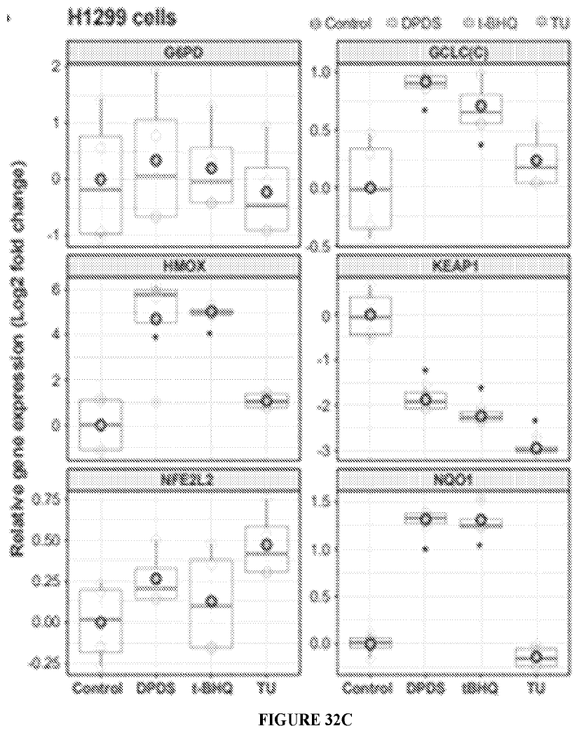

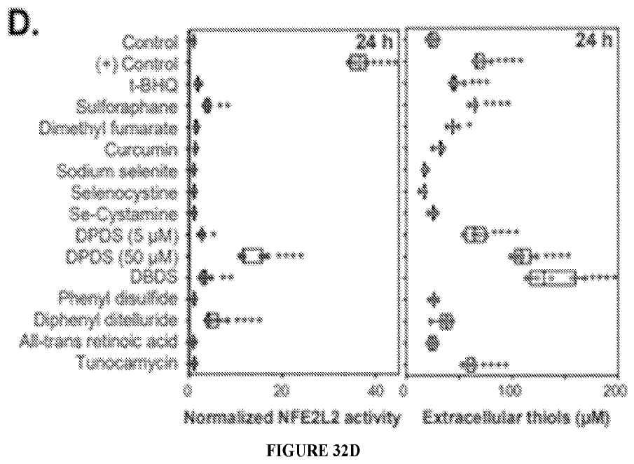

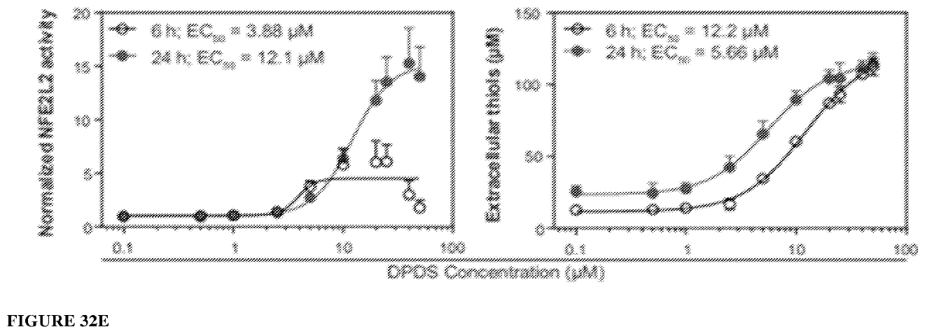

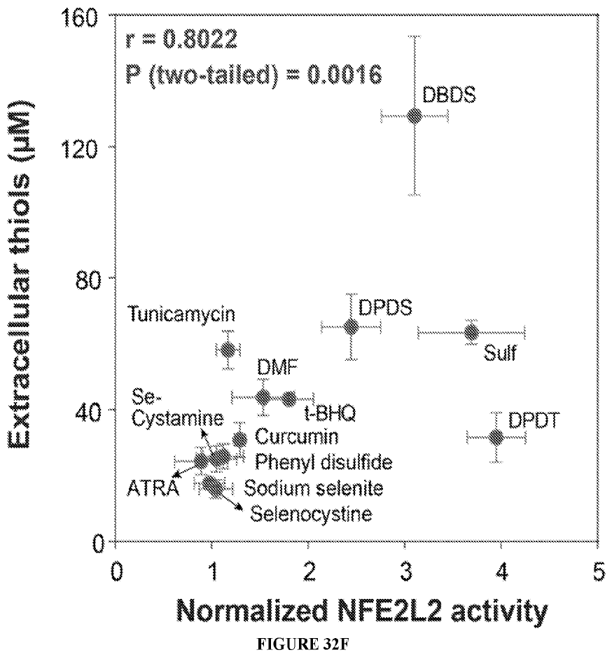

Transcription factor NFE2L2 regulates the expression of SLC7A11 upon binding to its promoter region¹³. Using a chemical biology approach, we interrogated whether cysteine efflux can be pharmacologically modulated by activation of NFE2L2 to modulate the extracellular redox potential. We used tert-butyl hydroquinone (tBHQ), a known NFE2L2 inducer, along with few other selected compounds in our studies. Among all the tested compounds, diphenyl diselenide (DPDS) and dibenzyl diselenide (DBDS) were found to be highly effective in increasing extracellular reduced thiols in all the tested cell lines comprising both epithelial (H1299) and myeloid lineages (HL60) (FIG. 32A). Such changes were associated with increased mRNA expression of NFE2L2 target genes by the selected compounds (FIG. 32B, 32C). The expression profile of the target genes differed depending on the test compounds and the cell lines, except that DPDS significantly induced the expression of HMOX and NQO1 at early time point. The involvement of NFE2L2 in the observed effects on the elevation of extracellular thiols by these small molecules was further investigated using a reporter cell line. We screened additional small molecules along with the ones outlined above. We found that treatment with these molecules had varying effects on the extracellular reduced thiols levels and NFE2L2 activity (FIG. 32D). Among the tested molecules, DPDS was unique in that it exhibited a concomitant dose-dependent increase in both the endpoints with a saturable kinetics (FIG. 32E). Correlation analysis suggested a strong association (Spearman r=0.80; p=0.0016, two-tailed) between the extracellular thiols and NFE2L2 activity (FIG. 32F). Such statistical inference could not explain certain discordance as follows. DPDT- and tBHQ-induced increase in extracellular reduced thiols levels was not incremental to their corresponding NFE2L2 activity. The opposite was true for tunicamycin treatment. These findings demonstrate that NFE2L2 activity is an important mediator of maintaining the redox potential of extracellular milieu, but indicate the involvement of additional pathways in regulating this key biological pathway.

### Roles of ATF4 in SLC7A11 Expression and Redox Control of Extracellular Milieu

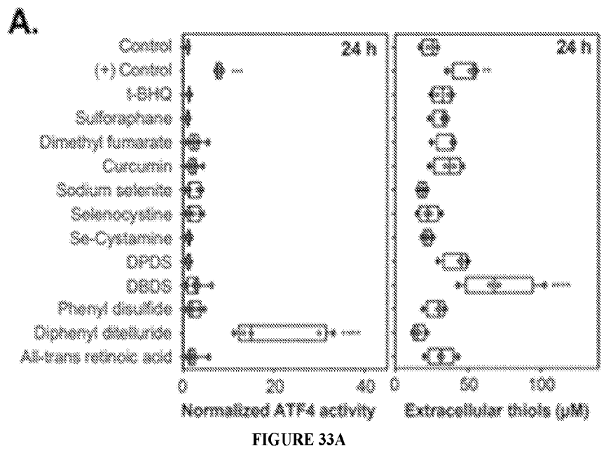

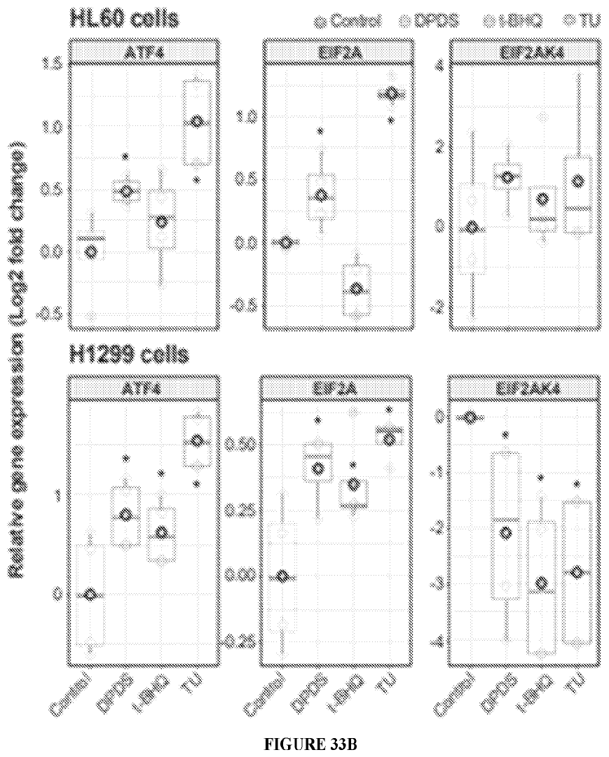

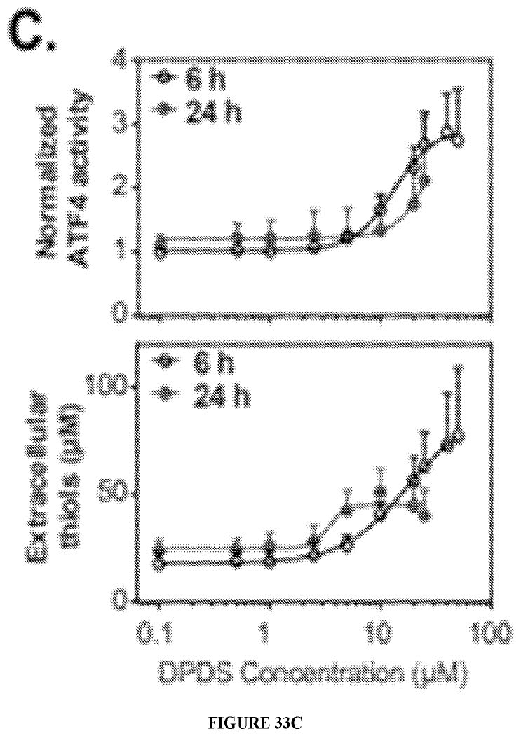

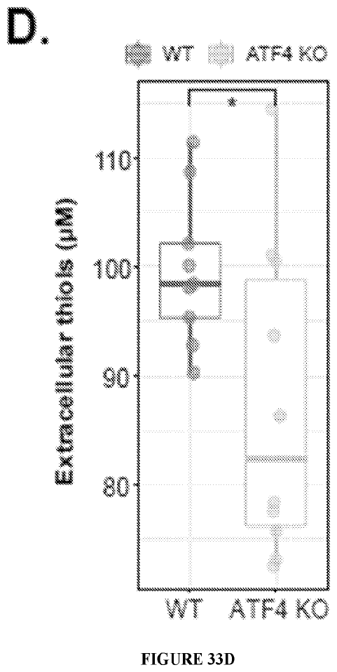

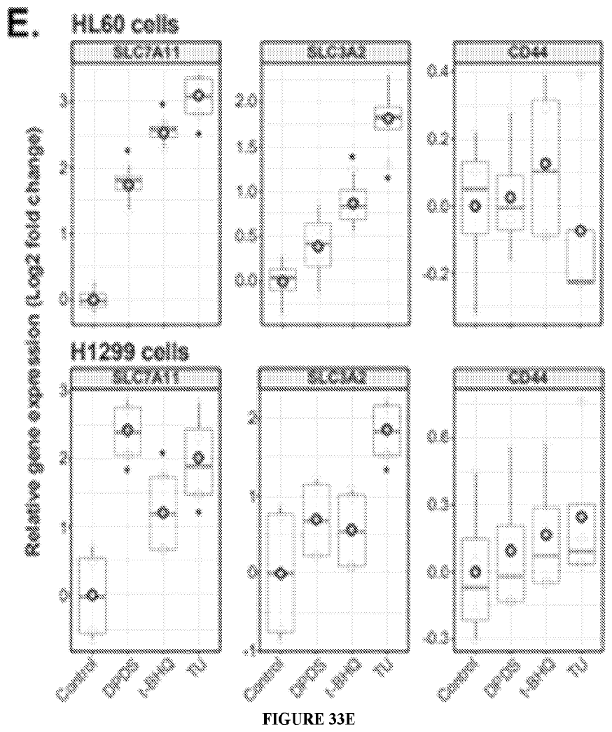

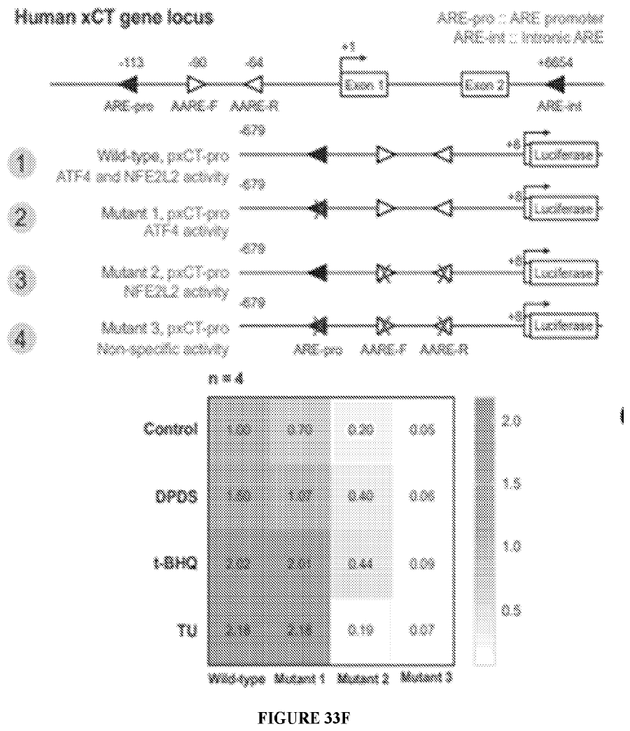

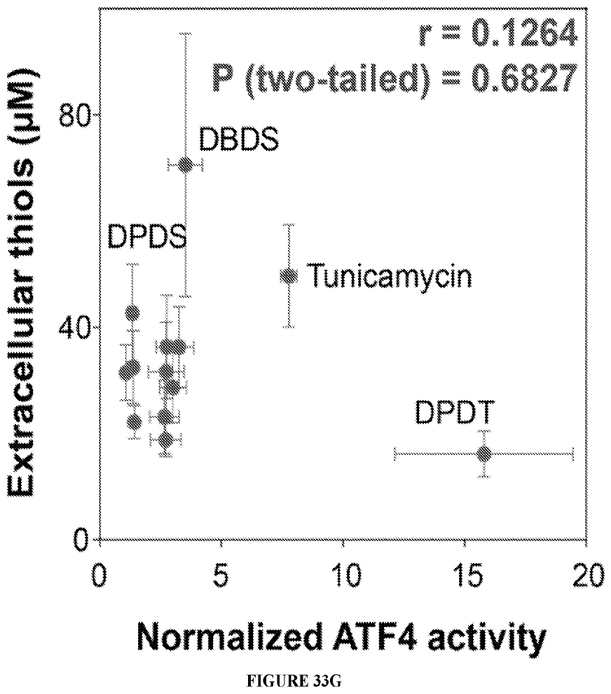

Three independent lines of evidence encouraged us to investigate the potential roles of ATF4 in the regulation of extracellular thiols by these small molecules. First of all, ATF4 is also a known regulator of SLC7A11 expression beyond the established roles of NFE2L213. Secondly, increased activity of ATF4 is implicated in amino acid stress response¹⁶. We hypothesized that the excess cystine influx by SLC7A11 could indirectly activate ATF4 by amplifying amino acid stress response. Finally, treatment with tunicamycin, a known agonist of ATF4, resulted in increased levels of extracellular reduced thiols. To determine whether ATF4 is functionally involved in modulating extracellular redox potential, HEK293 cells stably expressing ATF4-inducible luciferase construct were treated with these small molecules, as outlined in FIG. 32. Increased ATF4 activity was only recorded in the positive control (tunicamycin, 2.5 μM) and diphenyl ditelluride (5.0 μM) treated samples (FIG. 33A). This finding is supported by increased ATF4 expression in tunicamycin treated HL60 and H1299 cells (FIG. 33B). Except for the positive control, increased levels of extracellular reduced thiols were only found in the DBDS (5.0 μM) treated cells (FIG. 33A, right panel). In view of the earlier findings with NFE2L2 reporter MCF7 cells, no changes in the redox potential of the extracellular milieu by DPDS in HEK293 cells was unanticipated. However, further dose-response study revealed that the activation of ATF4 and concomitant increase in extracellular thiols necessitated higher doses of DPDS in these cells (FIG. 33C). In addition, we also demonstrate that extracellular reduced thiols levels were low in ATF4 knock-out HeLa cells (FIG. 33D), suggesting a direct role of ATF4 in regulation of extracellular reduced thiols via SLC7A11 expression.

Sulforaphane or t-BHQ that induced extracellular reduced thiols levels in MCF7 cells, were not effective in increasing ATF4 transcriptional activity in HEK293 reporter cells. In addressing this, we pursued further experiments to study the activity of both the transcription factors using a single cell type to eliminate cell-autonomous differences in the response to the treatment with the test compounds. Increased SLC7A11 expression in HL60 and H1299 cells following treatment with DPDS, t-BHQ and tunicamycin (FIG. 33E) support this hypothesis. We studied the functional involvement of the selected test compounds on augmenting NFE2L2- and ATF4-mediated regulation SLC7A11 expression. To do this, we transfected H1299 cells with human xCT gene promoter luciferase reporter plasmids and treated these cells with DPDS, t-BHQ, and TU. The latter two served as positive regulators of NFE2L2 and ATF4 activity, respectively. The wild-type plasmid contained both of NFE2L2 and ATF4 binding sites, while mutants were defective in either NFE2L2, ATF4 or binding sites of both of these transcription factors (FIG. 33F, top panel). Based on the relative luciferase activity data, ATF4 appeared to exert higher control over SLC7A11 expression in our experimental setting independent of the chemical properties of the test compounds (FIG. 33F, bottom panel). Additionally, no factual association between ATF4 activity and extracellular thiols (FIG. 33D) could be established (Spearman r=0.13; p=0.6827, two-tailed) in contrast to the earlier findings with NFE2L2. Overall, these data demonstrate that small molecules augmentation of NFE2L2 and ATF4 transcriptional activity increases SLC7A11 expression that is associated with a reducing extracellular milieu.

### Cystine Import Regulates Cysteine Efflux in the Local Microenvironment

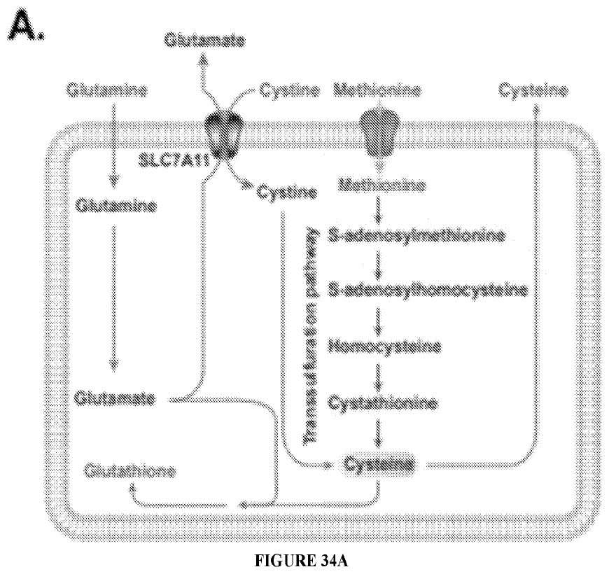

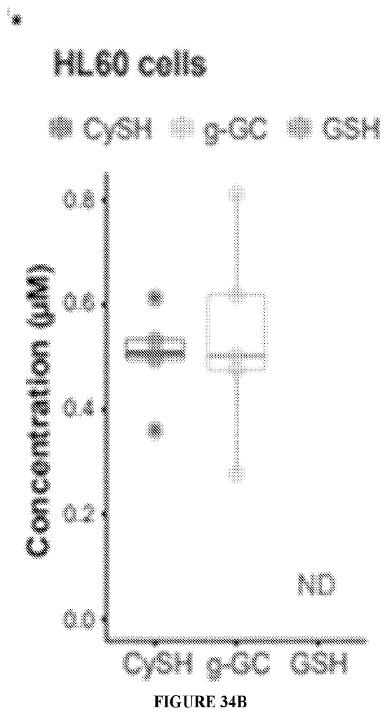

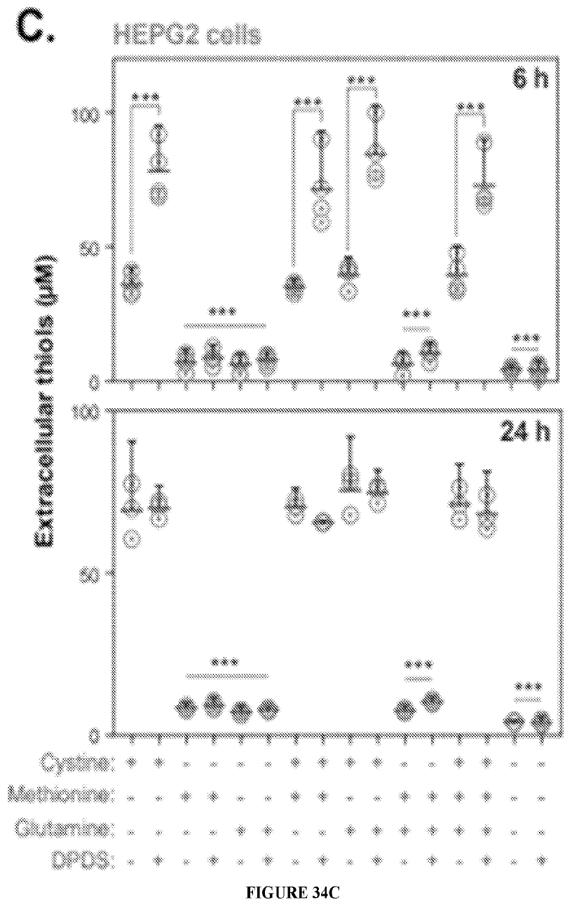

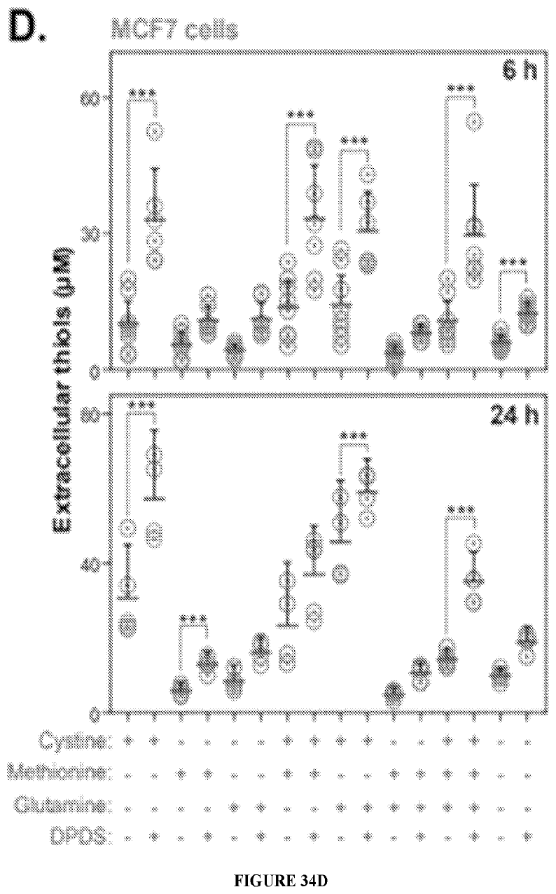

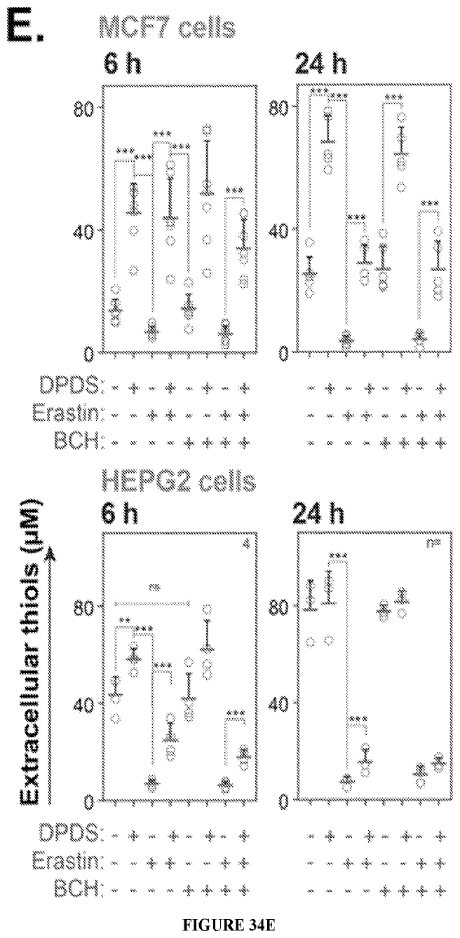

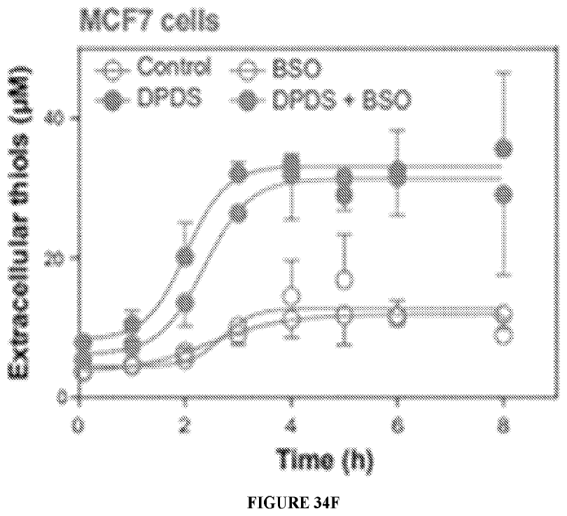

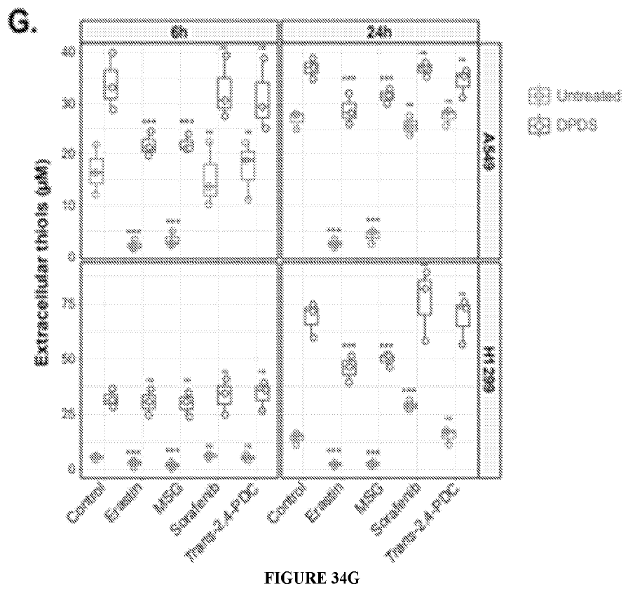

Cysteine is one of the most abundant non-protein thiols in the plasma. We hypothesized that SLC7A11-mediated cystine transport plays a key role in regulating the redox potential of extracellular milieu in cancer cells by effluxing cysteine in a cyclic process. A single step intracellular conversion of cystine to cysteine is an energy-efficient process compared to biosynthesis of cysteine via the transsulfuration pathway (FIG. 34A). HPLC-based analysis of the major non-protein thiols revealed cysteine and gamma-glutamyl cysteine as the major extracellular thiols in HL60 cells (FIG. 34B). Further mechanistic studies were focused on DPDS, as this compound was effective in increasing extracellular thiols in association with NFE2L2- and ATF4-mediated increase in SLC7A11 expression. To study the roles of cystine, glutamine and methionine in maintaining extracellular cysteine/cystine redox potential, we used culture medium free of these amino acids that were additionally supplemented at specified combinations. We initially employed HepG2 cells that are defective in transsulfuration pathway to address the importance of cystine import in maintaining extracellular reduced thiols levels. When these cells were cultured in medium depleted with cystine, extracellular reduced thiols levels were very low (FIG. 34C). Whenever cystine was available for uptake, extracellular reduced thiols levels returned to the baseline with no additional augmentation of thiols levels by DPDS at a later time point, suggesting SLC7A11-mediated cystine transport operates at its maximum capacity in this cell line. Similar results were found in MCF7 cells, albeit with a major difference in that methionine partially compensated in maintaining extracellular thiols levels, likely by cysteine biosynthesis via transsulfuration pathway (FIG. 34D). Importantly, the availability of cystine alone was sufficient to maintain extracellular thiols levels that was comparable when all three amino acids were available for transport.

We further interrogated whether the degradation of intracellular GSH plays any roles in elevating extracellular thiols. In co-exposure experiment, we demonstrate that inhibition of GSH biosynthesis by BSO had little or no impact on the DPDS-induced increase in thiols efflux in a time-course study (FIG. 34F). In subsequent pharmacological studies, we found that Erastin and mono-sodium glutamate inhibited thiols efflux, while sorafenib or glutamate transport inhibitor trans-2,4-PDC had no effect (FIG. 34F). Results from these studies indicate that the cystine is the major contributor of maintaining extracellular redox potential, while methionine plays a minor role only when cystine availability is limited.

### SLC7A11 Expression and Cystine Availability are Key Mediators of Sodium Selenite and Selenocystine Cytotoxicity in Cancer Cells

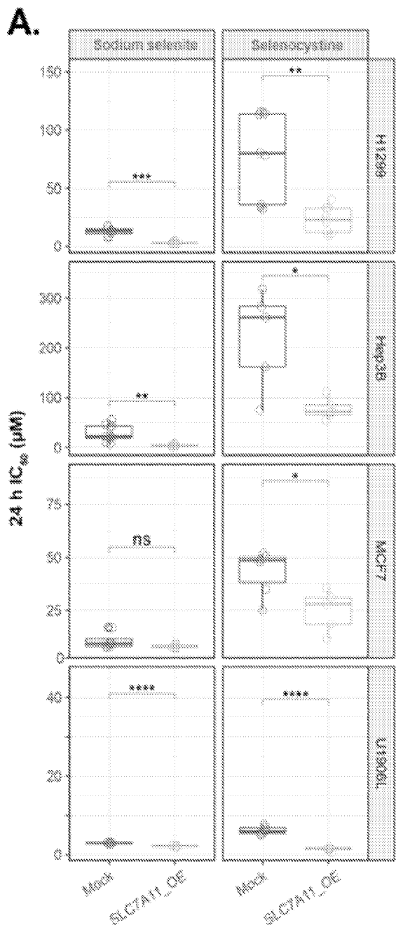

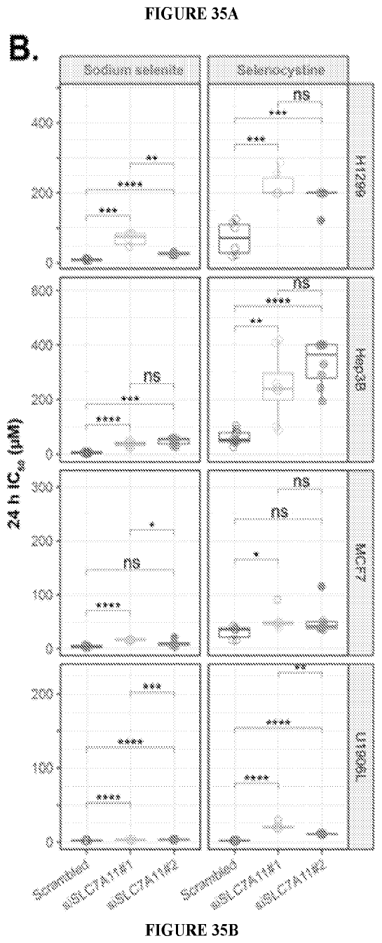

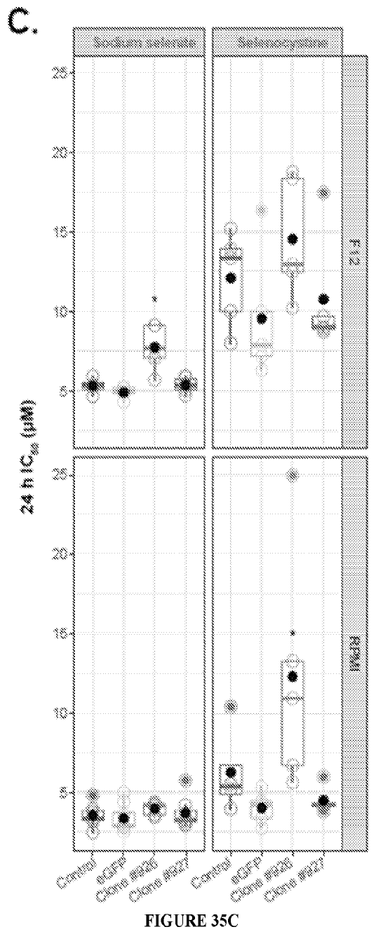

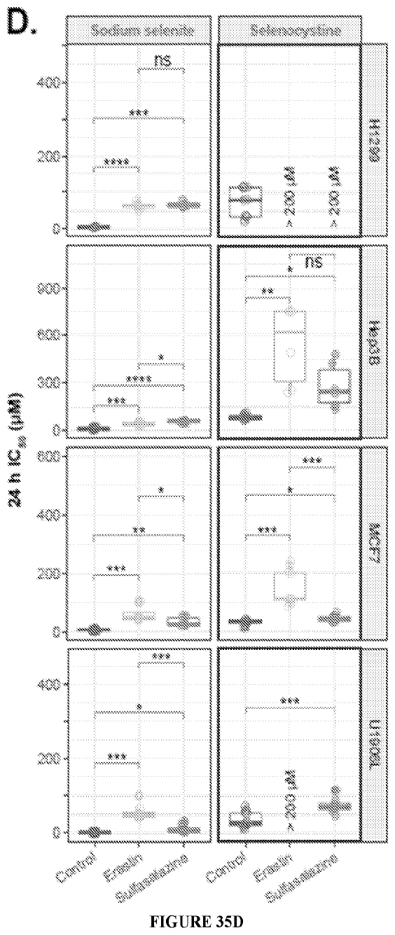

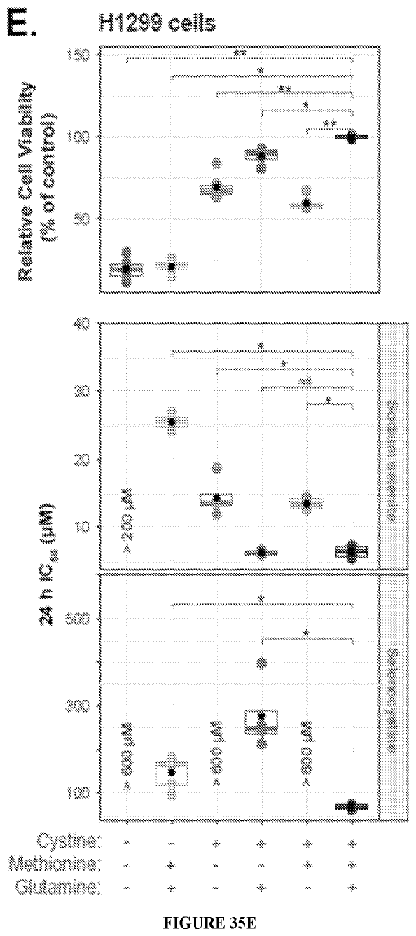

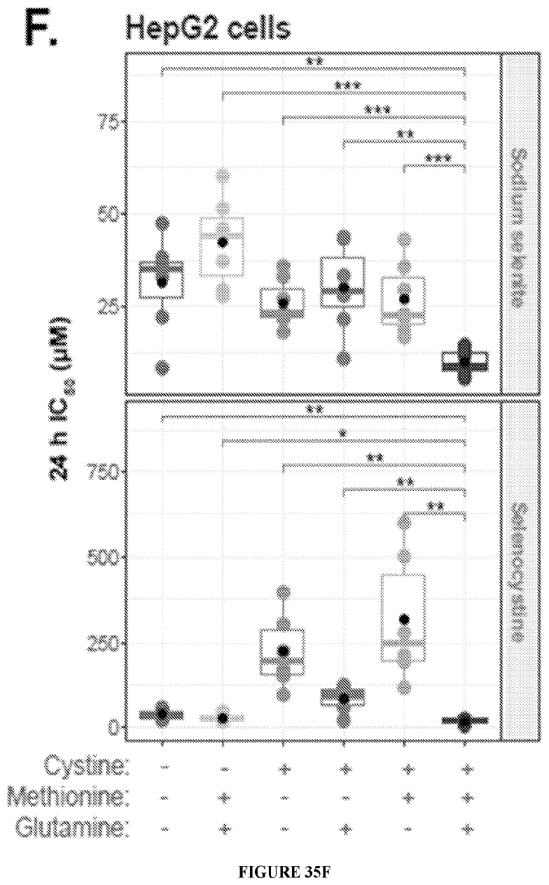

Findings outlined in FIGS. 31-34 suggest that small molecules-induced augmentation of cystine uptake is important for modulating the redox potential of local milieu. Such phenomenon presents a unique therapeutic opportunity in cancer using sodium selenite and selenocystine as experimental chemotherapeutics. Using radiotracer technique, a previous study has demonstrated that selenium uptake from selenite is significantly increased upon modulation of extracellular redox potential by exogenous addition of cysteine or GSH11. Similarly, selenocystine can possibly be transported via SLC7A11 due to structural similarity between its cognate substrate cystine with selenocystine. In connection, we hypothesized that of the induction of SLC7A11 activity may increase the cytotoxicity of sodium selenite and selenocystine in cancer cells and vice versa. We used 6 different cell lines to test this hypothesis. We demonstrate that plasmid-based overexpression of SLC7A11 resulted in increased cytotoxicity of sodium selenite and selenocystine to cancer cell (FIG. 35A). An opposite result was found following siRNA-based transient knock-down of SLC7A11 expression (FIG. 35B). Similarly, knock-down of SLC7A11 abrogated sodium selenite cytotoxicity in A549 cells stably transfected with shRNA clone #926 (FIG. 35C, top left panel) when cultured in F12 medium, mimicking to the extracellular thiols data (FIG. 31H). When these cells were cultured in high cystine- and glucose-containing RPMI medium, no such effects were observed except that control cells were highly sensitive to sodium selenite treatment (FIG. 35C, bottom left panel). For selenocystine, we found inhibition of cytotoxicity in clone #926 when cultured in RPMI medium but only a non-significant decrease in cytotoxicity in these cells cultured in F12 medium. We further wanted to find out whether chemical inhibition of cystine transport would abrogate toxicities of these selenium compounds mimicking the extracellular thiols data as outlined in FIG. 31F. Co-treatment with Erastin and Sulfasalazine resulted in inhibition of cytotoxicity of sodium selenite and selenocystine (FIG. 35D). Next, we investigated the roles of cystine, methionine, and glutamine availability on the cytotoxicity of sodium selenite and selenocystine, as previously shown for extracellular reduced thiols experiments (FIG. 34C-34E) except that we didn't use DPDS in these experiments. Serum-starved synchronous cell populations were used for this series of experiments. Short-term removal of cystine or methionine had significant impact on the viability of H1299 cells, specifically when cystine was unavailable (FIG. 35E, top panel). However, lack of cystine in the culture medium greatly reduced sodium selenite cytotoxicity (FIG. 35E, bottom panel). Methionine availability partially restored the cytotoxic effects. Glutamine was also found to be important for the cytotoxic effects of sodium selenite in the presence of cystine. For selenocystine, lack of all three amino acids or the absence of glutamine abrogated the cytotoxic effects of selenocystine (FIG. 35E, bottom panel). Similar to sodium selenite, methionine availability partly recuperated the cytotoxic effects of selenocystine in the absence of cystine. However, mean IC50 value was significantly higher when compared to the sensitivity to selenocystine in the presence of all three amino acids. We also used hepatocellular carcinoma-originated HepG2 cells to study these effects. We hypothesized that lack of cysteine biosynthesis via transsulfuration pathways would partially protect these cells from sodium selenite cytotoxicity when cystine is unavailable. Results from cytotoxicity study corroborates our hypothesis (FIG. 35F, top panel). Additionally, we found that these cells were less reliant on exogenous glutamine to exert toxic effects unlike H1299 cells. In the absence of glutamine, selenocystine was less toxic in both cell lines whenever cystine was present, possibly suggesting selenocystine transport via SLC7A11 or another cystine transporters. A significantly higher cytotoxicity in the absence of all three amino acids or even in the presence of methionine and glutamine supports the above in transsulfuration pathway-deficient HepG2 cells. When we interrogated the cytotoxic effects of these compounds in ATF4 KO HeLa cell line, no differences in toxicity of these compounds could be found when compared to its wild-type counterpart (data not shown). These results together demonstrate that SLC7A11 expression and cystine availability are very important for sodium selenite and selenocystine cytotoxicity in cancer cells.

### Modulation of Cysteine Redox Buffer of the Local Milieu in Cancer Cells is Therapeutically Tractable

We have earlier demonstrated that increased NFE2L2 and ATF4 activity results in increased expression of SLC7A11 with concomitant increase in extracellular reduced thiols. In subsequent experiments, we have also shown that these conditions are highly favorable to augment the cytotoxic effects of sodium selenite and selenocystine to cancer cells. We hypothesized that the combinatorial treatment of NFE2L2 and ATF4 inducers with sodium selenite and selenocystine would further exacerbate the cytotoxicity and would result in increased cell death. To test this, we used a set of small molecules, including DPDS, as mentioned earlier in our study. We initially measured the cytotoxicity of these small molecules in different cell lines. Non-toxic doses of these compounds were used for combinatorial cytotoxicity studies with sodium selenite and selenocystine.

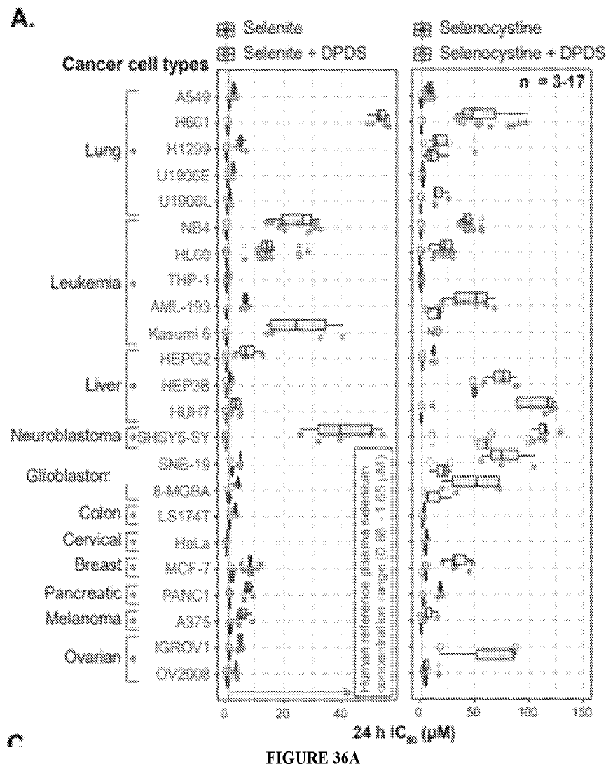

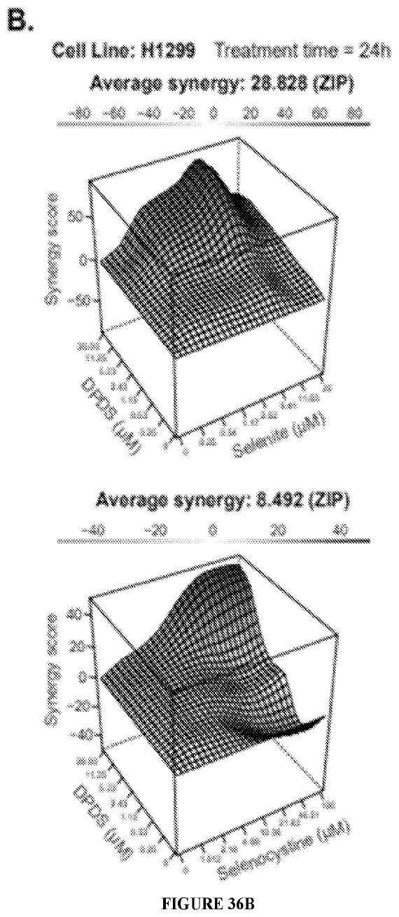

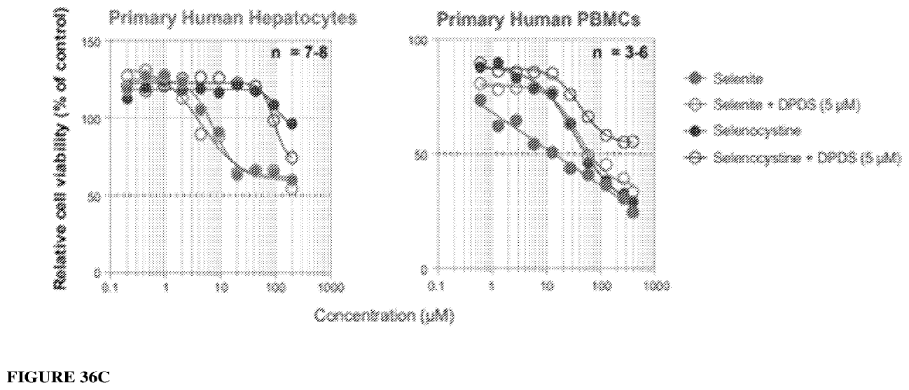

Findings from the initial cytotoxicity screening studies indicated that the combinations of DPDS with either sodium selenite or selenocystine resulted in superior cytotoxicity in all the tested cell lines. In following studies, we comprehensively measured the cytotoxicity of sodium selenite and selenocystine alone or in combination with DPDS at non-toxic concentration in 23 different human cell lines of multiple cancer origins. Corroborating to our hypothesis, the presence of DPDS further exacerbated the cytotoxicity of sodium selenite and selenocystine in all the cell lines without an exception (FIG. 36A). In certain cell lines, IC50 values for sodium selenite and selenocystine were below 1.0 μM in the combinatorial treatments. To understand the nature of the interactive effects, we measured cytotoxicity in HL60 and H1299 cell lines using different concentrations of sodium selenite, selenocystine, and DPDS. DPDS synergistically augmented the cytotoxicity of sodium selenite and selenocystine in both the cell lines (FIG. 36B). Synergy score suggested that the combination of DPDS with sodium selenite had greater impact on cytotoxic effects compared to its combination with selenocystine (FIG. 36B). When we tested the same combinations in primary human hepatocytes and peripheral blood mononuclear cells (PBMCs), we didn't observe any differences between the cytotoxic effects of the aforementioned single or combination treatments in these cells, indicating a plausible therapeutic window exists (FIG. 36C). Together, these data demonstrate that the combined treatment of DPDS with either sodium selenite or selenocystine harbor reasonable potential in therapeutic targeting of cancer cells.

### Discussion

Dysregulation of redox regulatory pathway is a common adaptive cell survival mechanism of cancer cells wherein it is under tight physiological control in normal cells7. Oxidative metabolism of cancer cells renders these susceptible to further exacerbation of oxidative stress that forms the basis for redox-directed cancer chemotherapy17. The central theme of this approach entails mounting oxidative damage to cancer cells by therapeutic interventions. Several approaches have been undertaken so far to achieve the same goal, but yet without any demonstrated success. One of the classical examples is the lack of responsiveness to SLC7A11 inhibitor sulfasalazine in cancer treatment¹⁸. At the inception of our study, we weighed on the rationale that the vulnerability to any redox-reactive agents could be further amplified upon concomitant augmented metabolic transformation of these agents by induction of redox-regulatory metabolic pathways. This strategy was predicted to be an efficient way compared to inhibition of specific redox-regulatory protein/s. In accordance, we herein demonstrate that augmented GSH biosynthesis and cysteine efflux upon induction of SLC7A11 is a plausible way to achieve this in human cancer cells of multiple tissue origins using sodium selenite and selenocystine as experimental redox-active agents. Similar paradigm possibly exists across any types of cancer cells that rely on SLC7A11 for cystine import in supporting adaptive redox equilibrium.

Our study revealed an important role of the redox potential of cancer cell microenvironment in determining the cytotoxic effects of sodium selenite and selenocystine in cancer cells. We experimentally demonstrated that SLC7A11 plays a key role in this process. Independent of cancer cell origins and subtypes, such observation was consistent across all the cell lines without an exception. Availability of sulfur-containing amino acids, mainly cystine, for uptake was central to the cytotoxic effects. This has further been experimentally validated using a transsulfuration pathway-defective cell line, HepG2. Given than cystine is present at relatively high concentration in plasma14 and available for oral supplementation, our findings offer how an endogenous metabolite can be harnessed for therapeutic gain when these selenium compounds are used as experimental cancer chemotherapeutics.

A number of small molecules have been identified that are activators of transcription factors NFE2L2 and ATF4 involved in the regulation of SLC7A11 expression upon binding with its promoter region. Treatment with these small molecules or nucleic acid-based induction of SLC7A11 was associated with increased influx of cystine, GSH biosynthesis, and cysteine efflux. Resulting changes in the redox potential of intracellular and extracellular microenvironment was found out to be highly favorable for sodium selenite and selenocystine cytotoxicity in cancer cells. DPDS, an activator of transcription factors NFE2L2 and ATF4, synergistically augmented the cytotoxicity of sodium selenite and selenocystine in an unprecedented manner. In certain cancer cell types, 24 h IC50 values for these small molecule selenium compounds were below 1.0 μM, comparable to that of normal plasma total selenium concentration in human. Notably, we didn't observe augmented cytotoxic effects of the combinatorial treatments in normal human hepatocytes and peripheral blood mononuclear cells. These preclinical studies suggest existence of a plausible therapeutic window that remained to be tested in the clinical settings.

Together, our study demonstrates how microenvironment redox control in cancer cells can be therapeutically targeted using a novel polypharmacology approach involving well studied redox-active small selenium molecules.

### Materials and Methods

### Cell Lines, Chemicals and Other Supplies

Information on cell lines, their seeding density and respective culture conditions are provided in the Supplementary Tables 1 and 2. All the cell lines were maintained in a humidified incubator at 37° C. with 5% C02 and tested regularly for mycoplasma contamination. All the cell lines were cultured in antibiotic-free condition unless mentioned otherwise. The information on all chemical compounds used in this study is provided in the Supplementary Table 3.

### Culture of Primary Human Hepatocytes

Primary human hepatocytes were cultured as reported elsewhere (PMID: 27598296). Briefly, isolated hepatocytes were seeded in collagen-coated (0.3 mg/ml) 96-well plates in Williams E medium containing 2 mM glutamine, 12 mM HEPES, 100 nM dexamethasone, 12 nM insulin, 0.01 M Gentamycin and 50 nM Amphotericin at 37° C. in a humidified incubator with 5% C02. Following 24 h initial acclimation period, the culture medium was changed and cells were treated with the test compounds for another 24 h. Cell viability was measured with WST-1 assay.

### Culture of Peripheral Blood Mononuclear Cells (PBMCs)

Human PBMCs were isolated from buffy coats obtained from Karolinska University Hospital, Huddinge from consented anonymous donors using density gradient centrifugation on Ficoll Paque Plus (GE Health Care, Sweden). Cells were cultured in complete RPMI 1640 media containing 1M HEPES, 5% FCS, 2 mM glutamine, 100 U/ml penicillin and 100 μg/ml streptomycin. Following 24 h initial acclimation period, half of the culture medium was carefully aspirated and cells were treated with the test compounds for another 24 h. Cell viability was measured with WST-1 assay.

### Proliferation/Cytotoxicity Assays

Two different cytotoxicity/proliferation kit was used in this study i.e., WST-1 proliferation assay kit (Roche, Indianapolis, IN, USA; Cat #28620000) and Cell-titer Glo-luminescent cell viability assay (Promega, WI, USA; Cat #0000348721). Majority of the cell proliferation/cytotoxicity experiments were performed with WST-1 assay. Briefly, cells were seeded at the indicated density for 24 h in 96 well plate, media was replaced, and subsequently treated with the test compounds at different doses. At termination of exposure, 90% of old culture media was replaced with fresh media and cells were incubated at 37° C. for 30-60 min depending on the cell types. Absorbance was measured at 430/630 nm using a spectrophotometer (PowerWave HT, BioTek, VT, USA). Cell titer Glo-assay was additionally performed in certain cases to validate the results from WST-1 assay as outlined above following manufacturer's instructions and luminescence was measured using a FLx100 Luminometer (CLARIOSTAR®, Ortenbery, Germany). In this case, cells were seeded in 96 well-Black plates (Thermo Scientific, Roskilde, Denmark; Cat #149692). Other proliferation/cytotoxicity assays were also carried out i.e., Quant™-iT PicoGreen ds DNA assay kit (Thermo Scientific, Rockwood, TN, USA, P7589) following manufacturer's instructions, Trypan blue (Sigma, Ayrshire, UK, T8154) and acid phosphatase assay for validation of cytotoxicity/proliferation data from other assays.

### Determination of IC50 and Synergy Analysis

For cytotoxicity assessment, test compounds were serially diluted in the respective culture media and cell proliferation/cytotoxicity was measured as outlined above. Dose-response curve (Variable slope, four parameters) was generated and the respective IC50 values were calculated using GraphPad Prism 8.3.0. For synergy analysis, an 8×8 dose matrices were used for this purpose with incremental doses of sodium selenite/selenocystine in combination with diphenyl diselenide. Synergistic effects were calculated using the ‘synergyfinder’ package in R.

### Extracellular Thiols Measurement

Cell culture media was collected at specified time points after incubation with the respective compounds as described in the respective figure legends and extracellular total thiols were measured using the DTDP method 19. The protocol was adjusted to 96-well plate format. Briefly, the assay mixture containing 1:3:5 ratio of DTDP:cell culture media:assay buffer (100 mM NaH2PO4 with 0.2 mM EDTA, pH5.8) was incubated in a 96-well UV transparent plate (Corning, ME, USA; Cat #27617048) for 5 min in room temperature. Absorbance was measured at 324 nm in a spectrophotometer (PowerWave HT, BioTek, VT, USA). The assay mixture with fresh media was used as a blank. Under the test condition, the molar extinction coefficient for 4-thiopyridone was 21,400 M⁻¹ cm⁻¹ and this value was used for the calculation of total thiols.

### NRF2, ATF4 and NF-kB Reporter Assays

NRF2 reporter MCF7 cells (Signosis, Santa Clara, CA, USA; Cat #SL-0010) were seeded (seeding density—600 cells/mm2 surface area) on to 96-well black plates (Thermo Scientific, Roskilde, Denmark; Cat #149692) for overnight in complete DMEM medium (Gibco, Paisley, PA, UK; Cat #2026757) containing 10% heat-inactivated FBS (Gibco, Paisley, PA, UK; Cat #08Q1095K). The next day, old media was replaced with media containing the respective compounds as described in the figure legends. Six and 24 h post-treatment, media was aspirated and replaced with 1:1 ratio of PBS:Bright-Glo luciferase reagent mix (Promega, Madison, WI, USA; Cat #0000418566) according to manufacturer's protocol. Cells were incubated in the dark for 10 min at room temperature with gentle rocking and luciferase activity was measured using an FLx100 Luminometer (CLARIOSTAR®, Ortenbery, Germany). The aspirated media was subjected to extracellular thiols measurement. In parallel, the same setup was made, and the cells were used to monitor the proliferation/toxicity of individual compounds. Luciferase signal was normalized to correct for the proliferation/toxicity of the individual compounds. The cells treated with 50 μM TBHQ (Sigma-Aldrich, St. Louis, MO, USA; Cat #112941) was used a positive control. For ATF4 reporter assay, Hek293T reporter cells 20 were cultured similarly as indicated for MCF7 cells, except for seeding density was set at 300 cells/mm2. All other experimental set up was similar. Cells treated with 2.5 μM tunicamycin (ApexBio, Houston, TX, USA, Cat #B7417) was used a positive control. NF-kB reporter Hek293T cells (BPS Bioscience, San Diego, CA, USA; Cat #60650) were seeded (seeding density—600 cells/mm2) on to 96-well black plates (Thermo Scientific, Roskilde, Denmark, 149692) for overnight with complete EMEM (ATCC, Middlesex, UK; Cat #30-2003) media and the transcriptional activity of NF-kB was measured as indicated above. Cells treated with 1 ng/ml of human recombinant IL-1β (BPS Bioscience, San Diego, CA, USA; Cat #90168) was used a positive control.

### xCT Reporter Assay

H1299 cells were seeded into 6-well plates in complete RPMI 1640 medium at a density of 400 cells/mm² for overnight. Next day cells were transfected with wild-type and mutant xCT promoter-luciferase reporter plasmid (Generously gifted by Dr. Junsei Mimura, Hirosaki University Graduate School of Medicine, Japan) using Lipofectamine 3000 (Invitrogen, Carlsbad, CA, USA, 2189652) according to the manufacturer's protocol. The plasmids were identified as pxCT-pro WT-Luc, pxCT-pro mtl-Luc (mutation in ARE promoter region), pxCT-pro mt2-Luc (mutation in AARE promoter region) and pxCT-pro mt3-Luc (mutation in ARE and AARE promoter region)¹³. Twenty four hour post-transfection, these cells were harvested and seeded at a density of 400 cells/mm2 on to 96-well black plates (Thermo Scientific, Roskilde, Denmark; Cat #149692). After 24 h, the media was replaced and replenished with media containing respective compounds as described in the figure legend. Six & 24 h post-treatment, luciferase activity was measured as outlined above. In parallel, the same setup was made, and the cells were used to monitor the proliferation/toxicity and extracellular thiols. Results were normalized to the proliferation/toxicity data. Cells treated with 10 & 20 nM of Bortezomib (Cell Signaling Technologies, Danvers, MA, USA; Cat #2204) was used a positive control.

### Overexpression and Knockdown Experiments

Cells were seeded into 6-well plates at a seeding density described in the table 7 and grown for 24 h. Two different SLC7A11 siRNAs (40 μmol each; Cat #s24290 & s24289) and control siRNA (40 μmol; Cat #AM4611) (all from Life Technologies, Carlsbad, CA, USA) were transfected using Lipofectamine 3000 (Invitrogen, Carlsbad, CA, USA, 2189652) in the presence of 10% serum according to manufacturer's protocol. For overexpression experiments, 1 μg of human SLC7A11 cloned plasmids; hxCT-IRES-Pur-141pCAG-3SIP and control vector 141pCAG-3SIP (kindly gifted by Dr. Marcus Conrad from Helmholtz Zentrum Munchen, Germany) was used to overexpress SLC7A11. Twenty four post-transfection, the cells were harvested and seeded on to 96-well plate (Falcon, Durham, NC, USA, 100003662) for subsequent treatments. Efficacy of the overexpression and knockdown of SLC7A11 in transfected cells was verified by RT-PCR and Western Blot.

TABLE 7
List of cell lines used in this study, their culture conditions, sources and seeding density.

| | | | | Seeding density |
|---|---|---|---|---|
| Cancer | | | | (cells/mm² |
| type | Cell line | Culture condition | Source | surface area) |
| Acute | THP-1 | RPMI + 10% | ATCC (American Type | 1200 |
| monocytic | | FBS + 50 μM beta | Culture Collection) | |
| leukemia | | mercaptoethanol | | |
| Acute | AML-193 | IMDM + 5% | ATCC (American Type | 1200 |
| monocytic | | FBS + 5 ng/ml | Culture Collection) | |
| leukemia | | GM-CSF + 5 μg/ml | | |
| | | Insulin + 5 μg/ml | | |
| | | transferrin | | |
| Acute | NB 4 | RPMI + 10% FBS | DSMZ (Braunschweig, | 600 |
| promyelocytic | | | Germany) | |
| leukemia | | | | |
| Acute | HL 60 | RPMI + 10% FBS | DSMZ (Braunschweig, | 600 |
| myeloid | | | Germany) | |
| leukemia | | | | |
| Myeloid | Kasumi-6 | RPMI + 20% fbs + | ATCC (American Type | 1500 |
| leukemia | | 2 ng/ml GM-CSF | Culture Collection) | |
| Non-small | A549 | F12 + 10% FBS | ATCC (American Type | 200 |
| cell lung | | | Culture Collection) | |
| carcinoma | | | | |
| Lung | H661 | RPMI + 10% FBS | https://www.sciencedirect.com/ | 200 |
| | | | science/article/pii/S0006295208001391 | |
| Lung | H1299 | RPMI + 10% FBS | ATCC (American Type | 200 |
| | | | Culture Collection) | |
| Lung | H727 | RPMI + 10% FBS | | 600 |
| Small cell | U1906-E | RPMI + 10% FBS | https://www.sciencedirect.com/ | 1500 |
| lung | | | science/article/pii/S0006295208001391 | |
| carcinoma | | | | |
| Small cell | U1906-L | RPMI + 10% FBS | https://www.sciencedirect.com/ | 1200 |
| lung | | | science/article/pii/S0006295208001391 | |
| carcinoma | | | | |
| Embryonic | HEK 293 | DMEM + 10% FBS | | 200 |
| kidney | | | | |
| Cervix | Hela | RPMI + 10% FBS | | 200 |
| Bone | SH-SY5Y | RPMI + 10% FBS | | 600 |
| marrow | | | | |
| Ovary | OV2008 | RPMI + 5% FBS, | | 200 |
| | | 10 mM HEPES, 1 mM | | |
| | | sodium pyruvate, | | |
| | | 1x essential AA | | |
| Ovary | IGROV 1 | RPMI + 10% FBS | Charles River | 300 |
| | | | laboratories (Virginia, | |
| | | | USA) | |
| Skin, | A375 | DMEM + 10% FBS | ATCC (American Type | 400 |
| malignant | | | Culture Collection) | |
| melanoma | | | | |
| Glioblastoma, | 8 MG-BA | EMEM + 20% FBS | Gifted by Dr. Lalit | 600 |
| brain | | | Rane, KI | |
| Brain, | SNB-19 | DMEM + 10% FBS | Gifted by Dr. Lalit | 600 |
| astrocytoma | | | Rane, KI | |
| Colon | LS174T | EMEM + 10% FBS | ATCC (American Type | 600 |
| | | | Culture Collection) | |
| Pancreas/duct | Panc1 | F12:DMEM + 10% | ATCC (American Type | 400 |
| | | FBS | Culture Collection) | |
| HCC_Liver | Hep3B | EMEM + 10% FBS | ATCC (American Type | 600 |
| | | | Culture Collection) | |
| HCC_Liver | HUH7 | EMEM + 10% FBS | Gifted (Camilla | 400 |
| | | | Pramfalk, KI) | |
| HCC_Liver | HepG2 | EMEM + 10% FBS | ATCC (American Type | 600 |
| | | | Culture Collection) | |

TABLE 8
Additional cell lines used in this study.

| Cell line | Culture condition | Source |
|---|---|---|
| MCF 7_Nrf2 | DMEM + 10% FBS | https://www.signosisinc.com/product/nrf2-are- |
| | | luciferase-reporter-mcf7-stable-cell-line-non- |
| | | profit-sl-0010-np |
| HEK293_ATF4 | DMEM + 10% FBS | http://elifesciences.org/content/elife/2/e00498.full.pdf |
| A549_C | F12 or | https://elifesciences.org/articles/27713 |
| | RPMI + 10% FBS | |
| A549_eGFP | F12 or | https://elifesciences.org/articles/27713 |
| | RPMI + 10% FBS | |
| A549_#926 | F12 or | https://elifesciences.org/articles/27713 |
| | RPMI + 10% FBS | |
| A549_#927 | F12 or | https://elifesciences.org/articles/27713 |
| | RPMI + 10% FBS | |
| HEK293_NF-κB | DMEM + 10% FBS | https://bpsbioscience.com/nf-kb-reporter-luc-hek293- |
| | | recombinant-cell-line |
| ATF4 KO | RPMI + 10% FBS | https://doi.org/10.1083/jcb.201702058 |

### Determination of Intracellular and Extracellular Thiols by HPLC

HPLC was performed as described by Luo et al. 21. Samples and standards were derivatized using monobromobimane (Calbiochem, La Jolla, CA) in sodium N-ethylmorpholine. Aliquots of the derivatized samples were filtered using a 0.22 μm Millipore filter. Glutathione (GSH), cysteine (CySH) and g-glutamylcysteine (7-GC) concentrations were then analyzed using high-performance liquid chromatography (HPLC). Both the total and reduced forms were measured. The HPLC separation, of the thiol-bimane adducts, was achieved on a column (4.5×150 mm) packed with 3-μm octadecyl-silica reversed-phase resin (Supelco, Inc., Bellefonte, PA) followed by fluorescent detection at excitation 394 nm and emission 480 nm (Millipore Co., Milford, MA). The system consisted of a Waters 625 LC pump system, a Waters 470 scanning fluorescence detector, and a Waters 717 Ultra WISP sample processor (Millipore, Milford, MA). Elution solvent A was 7.0% aqueous acetonitrile containing 0.25% acetic acid and perchloric acid; the final pH was adjusted to 3.76 with sodium hydroxide. Elution solvent B was 75% acetonitrile. The flow rate was 1.0 ml/min. The derivatives of GSH, CySH and 7-GC were then quantified based on peak areas and compared with standards. The CV for this method is 2.6%.

RNA Isolation and qRT-PCR

Total RNA was isolated using RNeasy plus mini kit (Qiagen, Hilden, Germany; Cat #157014660) according to the manufacturer's instruction. Isolated RNAs were treated with Turbo-DNA free kit (Invitrogen, Grand Island, NY, USA; Cat #00450957) to eliminate genomic DNA contaminations. RNA quantity and quality were measured using NanoDrop spectrophotometer ND-1000 (Saveen & Werner A B, Limhamn, Sweden). Two μg of total RNA was subjected to reverse transcription using the Ominscript RT kit (Qiagen, Hilden, Germany; Cat #205113). Gene expression was analyzed by amplifying 1 ng of cDNA using the CFX96 Real-time System (Bio-Rad, Hercules, CA, USA) with iQ-SYBR Green supermix (Bio-Rad, Hercules, CA, USA; Cat #170-88823). The amplification parameters were set at 95° C. for 4 min (1 cycle), 95° C. for 15 s, and 65.6° C. for 30 s (39 cycles). Gene expression values for each sample were normalized to the average of four housekeeping genes. Details of the qPCR primers used in this study is summarized in Table 9.

TABLE 9
Sequence of the qRT-PCR primers used
in this study.

| Oligo Name | Sequence (5′ to 3′) | SEQ ID No. |
|---|---|---|
| SLC7A11 FWD | AAATGTTAACGGGAGGCTGCC | SEQ ID NO: 1 |
| SLC7A11 REV | TTCCTGCTCCAATGATGGTGC | SEQ ID NO: 2 |
| ATF4 FWD | TTCGACTTGGATGCCCTGTTG | SEQ ID NO: 3 |
| ATF4 REV | CTTTCTGGGAGATGGCCAATTG | SEQ ID NO: 4 |
| G6PD FWD | CATCATCGTGGAGAAGCCCTT | SEQ ID NO: 5 |
| G6PD REV | GATCCTGTTGGCAAATCTCAGC | SEQ ID NO: 6 |
| EIF2AK4 FWD | AGAGCAGGAGCAACGTGAAATC | SEQ ID NO: 7 |
| EIF2AK4 REV | AGAGCCTCCATGTAGAATGGCA | SEQ ID NO: 8 |
| NFE2L2 FWD | CGACGGAAAGAGTATGAGCTGG | SEQ ID NO: 9 |
| NFE2L2 REV | CTGGGAGTAGTTGGCAGATCCA | SEQ ID NO: 10 |
| SLC3A2 FWD | GTGCTGGGTCCAATTCACAAG | SEQ ID NO: 11 |
| SLC3A2 REV | CACCCCGGTAGTTGGGAGTA | SEQ ID NO: 12 |
| GCLC[C] FWD | GCTGTCTCCAGGTGACATTCCA | SEQ ID NO: 13 |
| GCLC[C] REV | CGCTCCTCCCGAGTTCTATCAT | SEQ ID NO: 14 |
| REEP5 FWD | ATGCTGAAGTGTGGCTTCCTGT | SEQ ID NO: 15 |
| REEP5 REV | TCTGCAGTCTCTTTGGCCTTGT | SEQ ID NO: 16 |
| CD44 FWD | CCGGACACCATGGACAAGTTT | SEQ ID NO: 17 |
| CD44 REV | GTGGGCAAGGTGCTATTGAAAG | SEQ ID NO: 18 |
| EIF2A FWD | TTATGCCTGCCAAAGCGACA | SEQ ID NO: 19 |
| EIF2A REV | ATCCCACACTTCCATTTGTCCC | SEQ ID NO: 20 |
| G6PD FWD | CATCATCGTGGAGAAGCCCTT | SEQ ID NO: 21 |
| G6PD REV | GATCCTGTTGGCAAATCTCAGC | SEQ ID NO: 22 |
| NQO1 FWD | GGGTATCTTTCCAGGCTTCCCT | SEQ ID NO: 23 |
| NQO1 REV | TTCCCTAAGTGGCCTCTTGAGC | SEQ ID NO: 24 |
| HPRT1 FWD | TGGAGTCCTATTGACATCGCCA | SEQ ID NO: 25 |
| HPRT1 REV | CCGCCCAAAGGGAACTGATA | SEQ ID NO: 26 |
| HMOX1 FWD | TTCCTTACCGTGGGCACTGAAG | SEQ ID NO: 27 |
| HMOX1 REV | GAACTGAGGATGCTGAAGGGCA | SEQ ID NO: 28 |
| KEAP1 FWD | GCATCCACCACAACAGTGTGGA | SEQ ID NO: 29 |
| KEAP1 REV | CATTCGCCACTCGTTCCTCTCT | SEQ ID NO: 30 |
| GPI FWD | CAAATCTGGAACCCGTGTGGA | SEQ ID NO: 31 |
| GPI REV | TTGTGATGCAGACCCTTCCGT | SEQ ID NO: 32 |
| C1orf43 FWD | TGGAAGGCGTCGTTCTCCTTT | SEQ ID NO: 33 |
| C1orf43 REV | CCTACCTCGCGCAGAATTGTTC | SEQ ID NO: 34 |

### Determination of Protein Concentration

Harvested cells were washed twice with ice-cold PBS and were lysed in RIPA buffer (Sigma-Aldrich, St. Louis, MO, USA; Cat #R0278) in ice for 30 min in the presence of 1 mM phenylmethanesulfonyl fluoride (PMSF) (Sigma-Aldrich, St. Louis, MO, USA, P7626) and 1% Protease inhibitor cocktail mix (Sigma-Aldrich, St. Louis, MO, USA, P8340). Further, lysed cells were sonicated at 4° C. for 30 s with 1- to 2-s pulses (Chemical Instruments AB, Lidindö, Sweden). The lysed cells were centrifuged at 13,000 rpm for 10 min at 4° C., the supernatant was collected and protein concentration was determined by the Pierce™ Bicinchoninic acid (BCA) Protein Assay Kit (Thermo Fischer Scientific, Rockford, IL, USA; Cat #23228) according to the manufacturer's instruction using a spectrophotometer (PowerWave HT, BioTek, Winooski, VT, USA).

### Amino Acid Starvation

Cells were serum-starved for 24 h for cell cycle synchronization and supplied with complete media for another 2 passages. These cells were seeded on to 96-well plate (Falcon, Durham, NC, USA; Cat #100003662) (HL60—1000 cells/mm2, HEPG2 and MCF7 cells—600 cells/mm2) with complete media either RPMI or DMEM for overnight. Next day, complete media was replaced with media that differed in cystine, methionine or glutamine levels, totaling 8 different compositions. The final concentration of amino acids added to the reconstituted media is listed in the Table 10. The aspirated media at specified time points was subjected for extracellular thiols measurement. In parallel, the same setup was made, and the cells were used to monitor the proliferation/toxicity. Similar set up was used to study the selenite and selenocysteine cytotoxicity in HL60, HEPG2, and H1299 cells.

TABLE 10
Final concentration of cystine, methionine, and glutamine
in the culture media for amino acid starvation experiments.

| | RPMI 1640 | DMEM |
|---|---|---|
| Amino acid | (Concentration, mM) | (Concentration, mM) |
| L-Cystine (C) | 0.21 | 0.15 |
| L-Methionine (M) | 0.10 | 0.2 |
| L-Glutamine (G) | 2.0 | 2.0 |

### REFERENCES

1. Misra S, Boylan M, Selvam A, Spallholz J E, Björnstedt M. Redox-active selenium compounds—from toxicity and cell death to cancer treatment. Nutrients. 2015; 7(5):3536-3556.
2. Misra S, Wallenberg, M., Brodin, O., Bjornstedt, M. ed Selenite in Cancer Therapy In: Brigelius-Flohe R, Sies, Helmut ed. Diversity of Selenium Functions in Health and Disease. Vol. 38. Boca Raton: CRC Press; 2015.
3. Selvam A K, Björnstedt, Mikael., Misra, Sougat. Therapeutic potential of selenium compounds in the treatment of cancer. In: Michalke B, ed. Molecular and Integrative Toxicology: Springer; 2018.
4. Painter E P. The Chemistry and Toxicity of Selenium Compounds, with Special Reference to the Selenium Problem. Chemical Reviews. 1941; 28(2):179-213.
5. Seko Y, Imura N. Active oxygen generation as a possible mechanism of selenium toxicity. Biomedical and Environmental Sciences. 1997; 10(2-3):333-339.
6. Cairns R A, Harris I S, Mak T W. Regulation of cancer cell metabolism. Nat Rev Cancer. 2011; 11(2):85-95.
7. Gorrini C, Harris I S, Mak T W. Modulation of oxidative stress as an anticancer strategy. Nature reviews Drug discovery. 2013; 12(12):931-947.
8. Keibler M A, Wasylenko T M, Kelleher J K, Iliopoulos O, Vander Heiden M G, Stephanopoulos G. Metabolic requirements for cancer cell proliferation. Cancer & metabolism. 2016; 4:16-16.
9. Combs J A, DeNicola G M. The Non-Essential Amino Acid Cysteine Becomes Essential for Tumor Proliferation and Survival. Cancers. 2019; 11(5):678.
10. Wondrak G T. Redox-directed cancer therapeutics: molecular mechanisms and opportunities. Antioxidants &amp; redox signaling. 2009; 11(12):3013-3069.
11. Misra S, Kwong R W M, Niyogi S. Transport of selenium across the plasma membrane of primary hepatocytes and enterocytes of rainbow trout. Journal of Experimental Biology. 2012; 215(9):1491-1501.
12. Olm E, Fernandes A P, Hebert C, et al. Extracellular thiol-assisted selenium uptake dependent on the x(c)-cystine transporter explains the cancer-specific cytotoxicity of selenite. Proceedings of the National Academy of Sciences of the United States of America. 2009; 106(27):11400-11405.
13. Ye P, Mimura J, Okada T, et al. Nrf2- and ATF4-dependent upregulation of xCT modulates the sensitivity of T24 bladder carcinoma cells to proteasome inhibition. Mol Cell Biol. 2014; 34(18):3421-3434.
14. Turell L, Radi R, Alvarez B. The thiol pool in human plasma: the central contribution of albumin to redox processes. Free Radical Biology & Medicine. 2013; 65:244-253.
15. Muir A, Danai L V, Gui D Y, Waingarten C Y, Lewis C A, Vander Heiden M G. Environmental cystine drives glutamine anaplerosis and sensitizes cancer cells to glutaminase inhibition. eLife. 2017; 6:e27713.
16. Harding H P, Zhang Y, Zeng H, et al. An integrated stress response regulates amino acid metabolism and resistance to oxidative stress. Mol Cell. 2003; 11(3):619-633.
17. Wondrak G T. Redox-directed cancer therapeutics: molecular mechanisms and opportunities. Antioxidants & Redox Signaling. 2009; 11(12):3013-3069.
18. Shitara K, Doi T, Nagano O, et al. Phase 1 study of sulfasalazine and cisplatin for patients with CD44v-positive gastric cancer refractory to cisplatin (EPOC1407). Gastric Cancer. 2017; 20(6):1004-1009.
19. Riener C K, Kada G, Gruber H J. Quick measurement of protein sulfhydryls with Ellman's reagent and with 4,4′-dithiodipyridine. Anal Bioanal Chem. 2002; 373(4-5):266-276.
20. Sidrauski C, Acosta-Alvear D, Khoutorsky A, et al. Pharmacological brake-release of mRNA translation enhances cognitive memory. Elife. 2013; 2:e00498.
21. Luo J L, Hammarqvist F, Andersson K, Wernerman J. Surgical trauma decreases glutathione synthetic capacity in human skeletal muscle tissue. Am J Physiol. 1998; 275(2):E359-365.

### Example 5

### Abstract

Dysregulated redox homeostasis is a hallmark of leukemia¹. Hyperproliferation and increased metabolic turnover of nutrients in leukemic cells is associated with elevated reactive oxygen species (ROS) production². While pro-tumorigenic functions of ROS are well-established, high ROS levels are cytotoxic beyond a certain threshold³. Elevated oxidative metabolism of leukemic cells switches the overall cellular redox milieu to an adaptive condition favorable for cell survival. However, such adaptation renders these cells highly vulnerable to cell death upon further exacerbation of oxidative stress accompanied by loss of equilibrium between cellular oxidation and reduction processes. Strategic elevation of redox stress presents a unique paradigm-challenging opportunity to target leukemic cells by redox-active small molecules³. The prognoses of AML are very poor, specifically in the relapsed or refractory disease. Development of new therapeutics for AML is an unmet need.

### Background

Leukemia is one of the leading causes of death among childhood cancer. In the United States, the age-adjusted incidence (per 100,000 resident population) of childhood leukemia (age <15 y) is 5.4 cases (2000-2017), while the mortality rate is 0.52 cases during the same period (Data Source: National Program of Cancer Registries). Thus, about one out of ten children die upon diagnosed with childhood leukemia, while many suffer from secondary complications arising out the treatments, including, but not limited to the recurrent disease and secondary malignancies. It still remains to be a major challenge to treat patients with either refractory or relapsed leukemia. AML has one of the worst prognoses among different subtypes of leukemia. Thus, the development of new therapeutics for the treatment of AML is a significant unmet need in the field. A comprehensive understanding of the pathophysiology of AML provides distinct opportunity to devise novel therapeutic strategies to treat this deadly disease. Phenotypically and genetically, AML is a group of highly heterogenous diseases^{10,11}, yet they share some common pathophysiological adaptations. A key observation is the relatively high ROS levels in mature leukemic cells with concomitant dysregulation of thiols redox homeostasis when compared to normal leukocytes¹². Similarly, LSCs are highly sensitive to redox stress when compared to normal hematopoietic stem cells (HSCs)^{13,14}. Such redox dysregulation is accompanied by high cysteine/cystine requirement⁵. Increased cysteine uptake fuels GSH biosynthesis that in turn protects leukemic cells from high basal levels of ROS and related oxidative damage-induced cell death. These common metabolic adaptations suggest that adaptive redox dysregulation is a vulnerability of leukemic blasts and LSCs and targeting the redox axis of cell survival could bring about effective therapeutic intervention.

AML is a group of hematological malignancies, characterized by abnormal accumulation of blast cells in the bone marrows and peripheral blood. AML progresses rapidly and is fatal within weeks or monthsunless treated. Existing repertoire of anti-leukemic drugs have markedly improved the disease outcome in these groups of patients. However, the rates of overall survival are far more sobering, highlighting the dismal outcome of relapsed leukemia following an initial response. In addition, primary and secondary drug resistance remains a pervasive issue. The complex and dynamic clonal architecture of AML are key drivers of variability in drug response and eventual emergence of secondary resistance in many patients.

Although targeting driver mutations and oncogenic signaling in cancer have considerable therapeutic advantages, the existence and emergence of genetically diverse clonal populations complicates treatment¹⁶. In fact, intra- and inter-tumor clonal heterogeneity is a common signature of malignancies, including AML¹⁷. This is further compounded by drug resistance¹⁸, an ancillary cell survival mechanism designed to evade the cytopathic effects of exogenous chemicals. A promising therapeutic approach to address such pathobiological complexity, as is the case with AML, is to identify and target critical metabolic vulnerabilities of mature blast cells and LSCs. The underling therapeutic strategies involves targeting the unique metabolic requirements of AML and its progenitor cells that confer selective survival advantages.

Such a unique target is the redox regulatory axis of endogenous thiols. Coupled with high ROS levels in mature leukemic cells, high levels of cystine/cysteine and methionine acquisition is an absolute requirement of fast proliferating leukemic cells¹⁹ to maintain homeostasis of intracellular thiols and allow for protein biosynthesis. This essential survival adaptation forms the basis for dysregulated redox homeostasis in leukemic cells that distinctly differs from the redox regulation of normal leukocytes and HSCs²⁰. The disruption of cystine/cysteine and methionine acquisition and their metabolic pathways in leukemic cells presents a target for therapeutic intervention due to absolute essentiality of sulfur-containing amino acids inredox regulation. Deprivation of these amino acids has been demonstrated as a useful strategy in targeting leukemia cells¹⁹. Limitations of such an approach are that 1) the levels of these amino acid in systemic circulation depends on dietary sources, and 2) malignant cells principally share all metabolic pathways as in normal cells, albeit with pathway-specific distinct pathophysiological adaptations. In addressing such caveats, a novel strategy is presented in this proposal that involves treatment-induced further elevation of basal ROS levels in leukemic cells accompanied by concomitant loss of cellular reducing equivalents. These changes collectively result in exacerbation of oxidative stress culminating into cell death.

The outlined two-pronged strategy is a development in the field of redox-directed experimental therapeutics. Aspects of specific interests are as follows. 1) Coupling of an amino acid transport with perturbation of redox homeostasis present plausible target specificity towards highly proliferating and nutrient addict leukemic cells. Although elevated amino acid requirement is common to highly proliferating normal cells, their redox-regulatory mechanisms are under tight physiological control. 2) The expression of SLC7A11 is a critical confounding factor for targeting leukemic cells using the adopted strategy. The identification of a class of agonists of SLC7A11 warrants overcoming the heterogeneity of SLC7A11 expression in leukemic cells. 3) LSCs are very sensitive to redox stress. At low-to-moderate oxidative stress, they emerge from quiescence and differentiate into mature leukemic cells that are highly sensitive to redox stress. The outlined strategy exactly addresses this premise. 4) Perturbation of redox homeostasis is a common feature across different malignancies beyond AML. Thus, this strategy has broader appeals and implications in the emerging field of redox-directed cancer therapeutics.

### Experimental

The studies described are designed to target a redox metabolism vulnerability, which is a common pathophysiological feature of leukemic cells. Previous studies demonstrate the effectiveness of this strategy in targeting redox metabolism of cancer cells. This therapeutic approach can be implemented in the context of a murine model of AML as a proof of principle to provide pre-clinical data for this therapy. Endogenous thiols, glutathione (GSH) and cysteine, are key regulators of the overall redox potential of intracellular and extracellular milieu, respectively. Their increased turnover is important for maintaining the pathophysiological redox homeostasis in leukemic cells. The antioxidant roles of GSH and cysteine were tested to determine if it can be strategically reversed by redox-active small molecules to attain therapeutic gain in a murine model of leukemia. Sodium selenite and selenocystine are two such molecules that exacerbate oxidative stress upon oxidizing GSH and cysteine with simultaneous ROS generation. In relation, a small molecule (diphenyl diselenide) was identified that concomitantly increase the intracellular GSH and extracellular cysteine levels by augmenting cystine import factors that are highly favorable for delivery and reinforcing the cytotoxic effects of sodium selenite and selenocystine⁴⁻⁹.

Tests can be performed to determine if diphenyl diselenide-induced turnover of cellular thiols can synergize with the cytotoxic effects of sodium selenite and selenocystine in mature leukemic cells and leukemic stem cells (LSCs) for therapeutic gain using a murine model of acute myeloid leukemia (AML) in vivo and in vitro. Also, the anti-leukemic effects and bone marrow toxicity of the individual and combined treatments of sodium selenite or selenocystine, with diphenyl diselenide in a murine model of AML can be examined. AML mice are generated by retroviral transduction of murine HSCs and subsequent transplantation of syngeneic LSCs into secondary recipients. For in vivo efficacy and bone marrow toxicity studies, experimental therapeutics can be delivered intraperitoneally at prespecified doses in secondary transplanted mice of either gender following IACUC protocol. Each treatment group can have at least 10 mice, yielding a statistical power of 0.824 with an effect size of 0.4 and a predefined R to a ratio of 1.

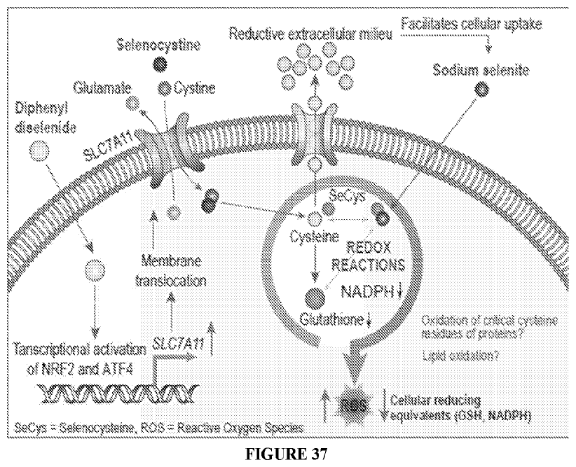

A strategy in which cellular thiols-based antioxidant systems are switched into prooxidative cues that induce robust redox imbalance, leading to cell death was developed (FIG. 37). This is achieved by simultaneous increase in intracellular GSH and extracellular cysteine levels by diphenyl diselenide (DPDS), a small-molecule that induces the expression of the cystine-glutamate exchanger (SLC7A11), implicated in inward cystine transport. Cystine is intracellularly reduced to cysteine to support GSH biosynthesis. Excess cysteine is effluxed out of the cells rendering a reductive extracellular milieu. Simultaneous induction of transcription factors (TFs), NFE2L2 and ATF4, by DPDS plays a key role in this process by positively regulating the expression of SLC7A11. However, individual combination of sodium selenite or selenocystine with DPDS at pharmacological concentrations results in unprecedented cancer cell death due to oxidations of thiols by these redox-reactive small molecules. Among all the cancer cell types tested, leukemic cells are found to be the most sensitive ones to the combination treatments. The anti-leukemic efficacies of these treatments were also demonstrated in a secondary subcutaneous xenotransplant mouse model of human leukemia. Mice receiving the combined treatments had significantly lower tumor volume when compared to the single treatments. Although these findings are highly reassuring in a highly aggressive mouse model of human leukemia, this model did not recapitulate key biological aspects of leukemia in which bulk of leukemic cells originate and reside in bone marrow and metastasize to multiple organs. New data can be generated aiding to understanding the efficacy of the outlined strategy of redox modulation in targeting AML cells. DPDS-induced modulation of cystine/cysteine transport in mature leukemic cells and LSCs can sensitize these cells to redox-active small molecules (sodium selenite or selenocystine) when used in combinations elicit severe redox imbalances. To study the anti-leukemic effects and bone marrow toxicity of single or combinatorial treatments of sodium selenite or selenocystine, in combination with DPDS in a syngeneic mouse model of AML.

The anti-leukemic effects of the single and combination treatments can be assessed in a mice model of AML expressing human fusion oncoprotein, MLL-AF9, following a specified dosing regimen. Mouse LSCs, harboring viral-transduced MLL-AF9 gene, can be engrafted in immunocompetent mice and secondary transplanted mice can be used to evaluate treatment efficacy and bone marrow toxicity (impacts on normal HSCs). The effects of these treatments on the distribution and frequency of LSCs can be evaluated in vivo. Self-renewal potential of the LSCs can be studied employing colony forming assays.

We can utilize an established and ongoing mice model of AML²³. Leukemia, being a stem cell disease, can be easily propagated by serial transplantation of leukemia stem cells. As reported earlier, these mice are generated by transplanting bone marrow-derived hematopoietic stem cells (HSCs, Lin-Sca1⁺cKit⁺) that are transduced with a lentivirus expressing the human fusion oncoprotein MLL-AF9. Stable genomic integration of the oncoprotein in HSCs converts these to LSCs with loss of Sca1 expression. When engrafted (6-8 weeks), LSCs repopulate in the bone marrow, proliferate, and can be used for secondary transplantation following cell sorting. The secondary recipient mice develop aggressive disease and can be subjected to in vivo efficacy assessment. To assess the treatment efficacy and bone marrow toxicity at the same time, LSCs generated from B6.SJL congenic mice that express the CD45.1 allele background can be transplanted into healthy CD45.2+ recipient mice, allowing us to distinguish donor LSCs from recipient HSCs (Lin-Sca1⁺cKit⁺CD45.2⁺).

Mimicking the aggressive treatment regimen in the clinical settings, secondary transplanted AML mice can be treated with the test molecules either as single agents or in combinations at specified dose levels every day for 14 days. In accordance, all test molecules can be injected (i.p.) daily in mice at non-toxic doses(sodium selenite and selenocystine: 1.5-3 mg/kg BW, DPDS: 4-10 mg/kg BW, range covers safety margin, refer FIG. 29 for exact dosing regimen) based on our preliminary dose escalation study in C57BL/6 mice. Besides, such high doses of sodium selenite is chosen based on allometric dose conversion from an earlier phase I clinical trial (NCT01959438) that has demonstrated a human tolerance limit of 10 mg sodium selenite/m² surface area/day when administered intravenously for two weeks. Disease progression can be monitored by enumerating complete blood counts (CBC), circulating mature leukemic cells by flow cytometry, and hematocrits weekly using peripheral blood. The experiment can be concluded 2 days after the final treatment.

A key focus of this study is to investigate whether these treatments affect quiescent LSCs, which contribute to post-treatment relapse. Earlier studies have reported that the LSCs are much more sensitive to oxidative damage than normal HSCs24. As the combined treatments induce severe redox stress in cancer cells, it can be tested whether the treatment-induced redox stress eliminates quiescent LSCs. Flow cytometry-based quantification of the LSCs (Lin-Sca1-cKit+ CD45.1+) abundance in the bone marrow, spleen, and peripheral blood in treated AML mice can be done. To measure the stemness and clonal expansion capacity of the residual LSCs, we can perform colony forming assay. Isolated Lin-cell populations from the treated mice can be used for this purpose. We already have an established protocol for this assay in which progenitor cells are grown in semi-solid medium under serum-deprived conditions containing a set of cytokines. In addition, if no circulatory leukemic cells can be found in a subset of animals following the treatments, their disease-free progression and survival can be monitored for another 3 months to assess the efficacy of the treatments in eradicating minimal residual disease that is a key contributor for disease relapse.

Normal hematopoiesis requires persistent turnover of bone marrow progenitor cells into lineage-specific blood cells. It is possible that high proliferation-associated increased metabolic turnover of cysteine/cystine may reinforce cytotoxic effects of the treatments to normal HSCs residing the leukemic niche. To assess this, the relative abundance and frequency of recipient CD45.2+ HSCs in bone marrow, spleen, and peripheral blood can be evaluated at treatment termination. In addition, redox-related plasma metabolites can be quantified to address the central hypothesis on systemic redox modulation by DPDS. The outlined experiments can determine whether a therapeutic window exists in which normal HSCs are spared from the toxic effects of the test molecules in the disease setting.

Our earlier animal study provides key information on the maximum tolerated dose levels of the test molecules in mice when used alone or in combinations. In drug combinations studies, finding the right therapeutic window is often challenging. To address this, in vitro (LSCs and HSCs) synergy analyses (6×6 dose matrix) can be performed to find the relative ratio of the test molecules resulting in the safest outcome on cytotoxicity in normal HSCs. Based on these findings, toxicity of the combined treatments on HSCs can further be tested in vivo in normal mice in a dose-escalation study to find a safe non-toxic dose regimen in vivo. Besides, the effectiveness of DPDS in inducing Slc7a11 is not known in mice mature leukemic cells and LSCs. In such a scenario, a fallback option can be to test already identified other small molecule inducers of SLC7A11. Alternatively, leukemia-bearing mice can be fed with cystine-rich diet prior and during the treatment with sodium selenite or selenocystine to address this paradox.

### Results:

SLC7A11-mediated cystine import is a key requirement for reinforcing the cytotoxic effects of selenite and selenocystine in cancer cells. Following findings from my unpublished studies provide several lines of experimental evidence in strong support of the proposed studies. Vector-based overexpression of SLC7A11 increases the cytotoxic effects of these redox-active molecules, while siRNA-mediated disruption of SLC7A11 expression or its pharmacological inhibition reverses their cytotoxic effects(FIG. 28G-28I). These effects were reproducible in all six tested cell lines, suggesting a common mechanism in which SLC7A11 plays a key role. Further studies demonstrated that DPDS, a small molecule with very low toxicity, was a potent inducer (out of several small molecules tested) of SLC7A11 expression via increased transcriptional activation of NRF2 and ATF4—transcription factors that control its expression (data not shown). DPDS-induced SLC7A11 overexpression was associated with increased levels of intracellular GSH, along with elevated levels of extracellular total thiols, mainly cysteine (FIG. 28J). Such global changes in the DPDS-induced intracellular and extracellular thiols pools are highly conducive for synergizing the cytotoxic effects of selenite and selenocystine²¹, as outlined above. This unique strategy was tested in 23 different cancers cell lines of multiple origins and overall mutational loads (data not shown). A synergistic increase in the cytotoxicity of selenite and selenocystine was observed in all the cells lines upon individual combinations with DPDS, without an exception. Of note, leukemic cells were highly sensitive to the combined treatments (FIG. 28A). For example, compared to selenite alone, a 107-fold increase in cytotoxicity(comparative 24 h IC50 values) was observed in Kasumi-6 cells following combined treatment of selenite and DPDS. Similarly, combined treatment of DPDS with selenocystine resulted in 102-fold increase in the cytotoxicity in NB cells when compared to selenocystine alone. Normal human hepatocytes and peripheral blood mononuclear cells (FIG. 28B) were highly resistant to these combination treatments at equivalent test concentrations, demonstrating a plausible therapeutic window in the preclinical settings. Toxicity studies in mice demonstrated a good safety profile of the combination treatments (data not shown). In vivo studies in a xenotransplant murine model of human leukemia demonstrated that the mice receiving the combined treatments had a significantly lower tumor volume when compared to the single treatments with sodium selenite, selenocystine, or DPDS alone (FIG. 28E). Notably, the combined treatments were highly effective in killing and inhibiting the proliferation of cancer cells ex vivo (unpublished data) in pancreatic cancer patients-derived tissue culture²², demonstrating their efficacies in targeting cognate human cancer cells beyond AML. In this backdrop, the following experiments are outlined to complement the set-out aim.

### LITERATURE CITED

1. Prieto-Bermejo R, Romo-Gonzalez M, Perez-Fernandez A, Ijurko C, Hernandez-Hernandez A. Reactive oxygen species in haematopoiesis: leukemic cells take a walk on the wild side. J Exp Clin Cancer Res. 2018; 37(1):125.
2. Pei S, Minhajuddin M, Callahan K P, et al. Targeting aberrant glutathione metabolism to eradicate humanacute myelogenous leukemia cells. J Biol Chem. 2013; 288(47):33542-33558.
3. Gorrini C, Harris I S, Mak T W. Modulation of oxidative stress as an anticancer strategy. Nat Rev Drug Discov. 2013; 12(12):931-947.
4. Misra S, Wallenberg, M., Brodin, O., Bjornstedt, M. ed Selenite in Cancer Therapy In: Brigelius-Flohe R, Sies, Helmut ed. Diversity of Selenium Functions in Health and Disease. Vol. 38. Boca Raton: CRC Press; 2015.
5. Misra S, Boylan M, Selvam A, Spallholz JE, Björnstedt M. Redox-active selenium compounds—from toxicity and cell death to cancer treatment. Nutrients. 2015; 7(5):3536-3556.
6. Selvam A K, Björnstedt, Mikael., Misra, Sougat. Therapeutic potential of selenium compounds in the treatment of cancer. In: Michalke B, ed. Molecular and Integrative Toxicology: Springer; 2018.
7. Wallenberg M, Misra S, Björnstedt M. Selenium cytotoxicity in cancer. Basic and Clinical Pharmacology and Toxicology. 2014; 114(5):377-386.
8. Wallenberg M, Misra S, Wasik A M, et al. Selenium induces a multi-targeted cell death process in addition to ROS formation. J Cell Mol Med. 2014; 18(4):671-684.
9. Misra S, Selvam A K, Wallenberg M, et al. Selenite promotes all-trans retinoic acid-induced maturation of acutepromyelocytic leukemia cells. Oncotarget. 2016; 7(46):74686-74700.
10. Estey E. Acute Myeloid Leukemia—Many Diseases, Many Treatments. N Engl J Med. 2016; 375(21):2094-2095.
11. De Kouchkovsky I, Abdul-Hay M. ‘Acute myeloid leukemia: a comprehensive review and 2016 update’. Blood Cancer J. 2016; 6(7):e441.
12. Lagadinou E D, Sach A, Callahan K, et al. BCL-2 inhibition targets oxidative phosphorylation and selectively eradicates quiescent human leukemia stem cells. Cell Stem Cell. 2013; 12(3):329-341.
13. Mattes K, Vellenga E, Schepers H. Differential redox-regulation and mitochondrial dynamics in normal and leukemic hematopoietic stem cells: A potential window for leukemia therapy. Crit Rev Oncol Hematol. 2019; 144:102814.
14. Yamashita M, Dellorusso P V, Olson O C, Passegue E. Dysregulated haematopoietic stem cell behaviour in myeloid leukaemogenesis. Nat Rev Cancer. 2020; 20(7):365-382.
15. Jones C L, Stevens B M, D'Alessandro A, et al. Inhibition of Amino Acid Metabolism Selectively Targets Human Leukemia Stem Cells. Cancer Cell. 2018; 34(5):724-740.
16. Vosberg S, Greif P A. Clonal evolution of acute myeloid leukemia from diagnosis to relapse. Genes Chromosomes Cancer. 2019; 58(12):839-849.
17. Grove C S, Vassiliou G S. Acute myeloid leukaemia: a paradigm for the clonal evolution of cancer? Dis Model Mech. 2014; 7(8):941-951.
18. Filipits M, Stranzl T, Pohl G, et al. Drug resistance factors in acute myeloid leukemia: a comparative analysis. Leukemia. 2000; 14(1):68-76.
19. Jones C L, Stevens B M, D'Alessandro A, et al. Cysteine depletion targets leukemia stem cells through inhibition of electron transport complex II. Blood. 2019; 134(4):389-394.
20. Testa U, Labbaye C, Castelli G, Pelosi E. Oxidative stress and hypoxia in normal and leukemic stem cells. Exp Hematol. 2016; 44(7):540-560.
21. Misra S, Kwong R W, Niyogi S. Transport of selenium across the plasma membrane of primary hepatocytes and enterocytes of rainbow trout. J Exp Biol. 2012; 215(Pt 9):1491-1501.
22. Misra S, Moro C F, Del Chiaro M, et al. Ex vivo organotypic culture system of precision-cut slices of human pancreatic ductal adenocarcinoma. Sci Rep. 2019; 9(1):2133.
23. Finch E R, Kudva A K, Quickel M D, et al. Chemopreventive Effects of Dietary Eicosapentaenoic Acid Supplementation in Experimental Myeloid Leukemia. Cancer Prev Res (Phila). 2015; 8(10):989-999.
24. Jones C L, Stevens B M, D'Alessandro A, et al. Inhibition of Amino Acid Metabolism Selectively Targets Human Leukemia Stem Cells. Cancer Cell. 2019; 35(2):333-335.

### Example 6

Suitable animal models to assay treatment with the sodium selenite, selenocystine, and diphenyl diselenide alone or in combinations can be used. For example, an animal model to assay treatment in liver cancer patients can be used. Animal model to assay treatment in liver cancer are known in the art. A sample model may be a mice with induced icteric hepatocellular carcinoma (HCC). Icteric hepatocellular carcinoma (HCC) may be induced by any suitable method known in the art. For example, as described by Singh, V. et al., Cell, 175, 679-694, 2018, dietary soluble fibers are fermented by gut bacteria into short-chain fatty acids (SCFA), which are considered broadly health-promoting. Accordingly, consumption of such fibers ameliorates metabolic syndrome. However, incorporating soluble fiber inulin, but not insoluble fiber, into a compositionally defined diet, induced icteric hepatocellular carcinoma (HCC). Such HCC was microbiota-dependent and observed in multiple strains of dysbiotic mice but not in germ-free nor antibiotics-treated mice. Furthermore, consumption of an inulin-enriched high-fat diet induced both dysbiosis and HCC in wild-type (WT) mice. Inulin-induced HCC progressed via early onset of cholestasis, hepatocyte death, followed by neutrophilic inflammation in liver.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A pharmaceutical composition comprising:
one or more cytotoxic agent(s), wherein the one or more cytotoxic agent(s) is selected from a compound of category 1, category 2, or a combination thereof;
one or more cytotoxicity enhancing agent(s), wherein the one or more cytotoxicity enhancing agent(s) is selected from a compound of category 3, category 4, category 5, a synthetic RNA encoding human SLC7A11 gene, a cystine/glutamate transporter protein encoded by human SLC7A11 gene or a combination thereof; and
one or more pharmaceutically acceptable carriers;
wherein the presence of the cytotoxicity enhancing agent increases the cytotoxicity of the composition by at least 1.5-fold compared to in the absence of the cytotoxicity enhancing agent.

2. The pharmaceutical composition of claim 1, wherein the cytotoxicity enhancing agent is present in an effective amount to increase the expression of human SLC7A11 gene.

3. The pharmaceutical composition of claim 1, wherein the cytotoxicity enhancing agent is present in an effective amount to increase the concentration of human cystine/glutamate transporter protein encoded by human SLC7A11 gene.

4. The pharmaceutical composition of claim 1, wherein the cytotoxicity enhancing agent is present in an effective amount to activate the overexpression of NFE2L2 or ATF4 transcription factors.

5. The pharmaceutical composition of claim 1, wherein the cytotoxic agent and cytotoxicity enhancing agent are present in the composition in a ratio of cytotoxic agent to cytotoxicity enhancing agent ranging from 100:1 to 1:100.

6. The pharmaceutical composition of claim 1, wherein the cytotoxic agent comprises a compound of category 1.

7. The pharmaceutical composition of claim 1, wherein the cytotoxic agent comprises a compound of category 2.

8. The pharmaceutical composition of claim 1, wherein the cytotoxic agent comprises a compound of category 1 and category 2.

9. The pharmaceutical composition of claim 1, wherein the compound of category 1 is sodium selenite, potassium selenite, selenium dioxide, sodium selenide, potassium selenide, selenious acid, elemental selenium, selenite anion, or hydrogen selenide, or a combination thereof.

10. The pharmaceutical composition of claim 1, wherein the compound of category 2 is: 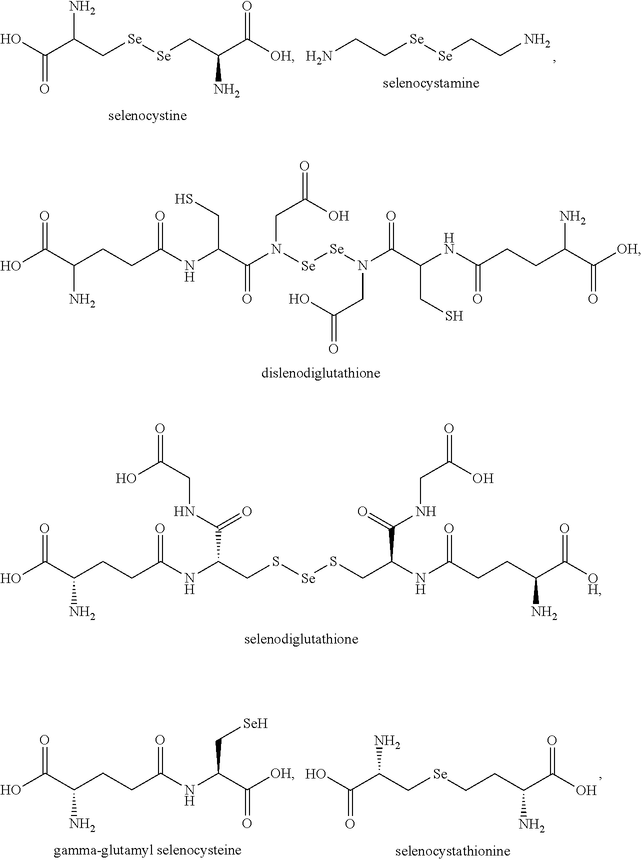
a pharmaceutically acceptable salt thereof, or a combination thereof.

11. The pharmaceutical composition of claim 1, wherein the one or more cytotoxicity enhancing agent(s) comprise a compound of categories 3, 4, 5, or a combination thereof, wherein the compound of categories 3, 4 or 5 is defined by Formula I:
R₃—X—Y—R₂ Formula I
or a pharmaceutically acceptable salt thereof, wherein
X is S, Se, or Te;
Y is absent, or is S, Se, or Te;
R₂ and R₃ are independently substituted or unsubstituted alkyl, aryl, heteroaryl, heteroalkyl, or a combination thereof,
wherein when present the alkyl is optionally substituted with an alkyl, aryl, primary amine, secondary amine, tertiary amine, primary amide, secondary amide, tertiary amide, carboxylic acid, thiol, carbonyl, hydroxy, ether, ester, halogen or a combination thereof;
wherein when present the aryl is optionally substituted alkyl, aryl, primary amine, secondary amine, tertiary amine, primary amide, secondary amide, tertiary amide, carboxylic acid, thiol, carbonyl, hydroxy, ether, ester, halogen, —CF₃, or a combination thereof.

12. The pharmaceutical composition of claim 1, wherein the compound of category 4 is methionine, S-adenosyl-L-methionine, selenomethionine, ethacrynate, salubrinal, proteasome inhibitors, bromsulphalein and its selenium analogues, Oltipraz, sulphoraphane, curcumin and its precursors and derivatives, dimethylfumarate, monomethyl fumarate, tert-butyl hydroxyquinone, diethyl maleate, small peptides that inhibits KEAP1 protein interaction with NRE2L2, or a combination thereof.

13. The pharmaceutical composition of claim 1, wherein the compound of Formula I is: 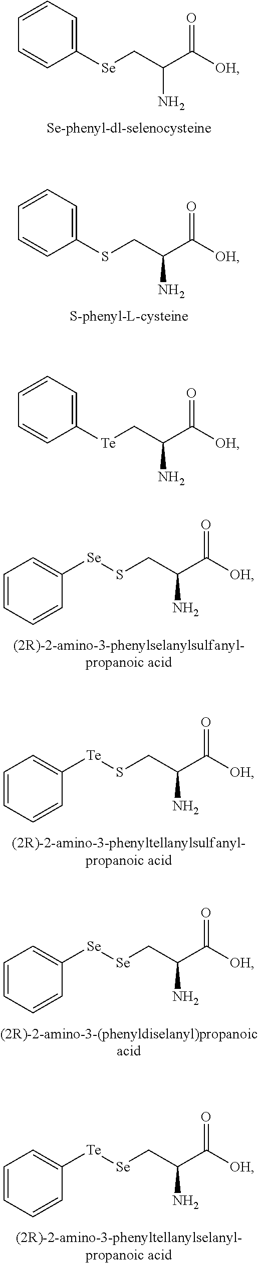 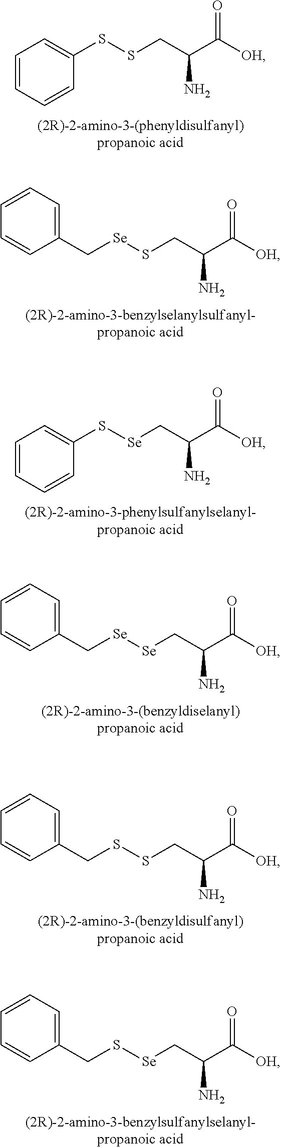 [SMILES: N[C@@H](CSSc1ccccc1)C(=O)O ; N[C@@H](CS[Se]Cc1ccccc1)C(=O)O ; N[C@@H](C[Se]Sc1ccccc1)C(=O)O ; N[C@@H](C[Se][Se]Cc1ccccc1)C(=O)O ; N[C@@H](CSSCc1ccccc1)C(=O)O ; N[C@@H](C[Se]SCc1ccccc1)C(=O)O] 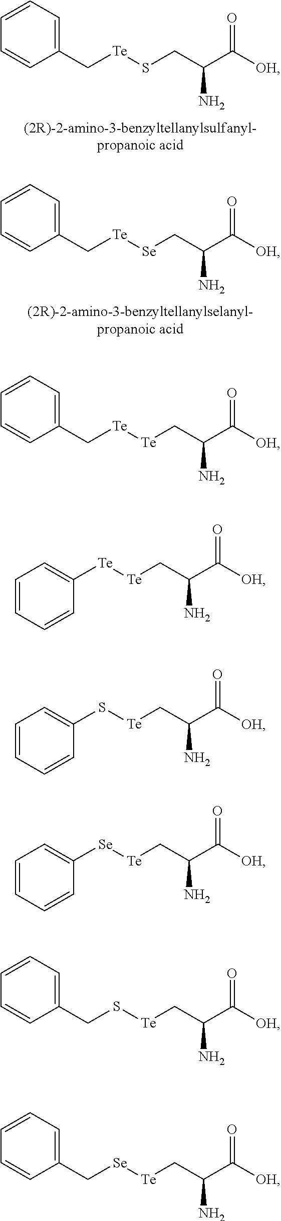 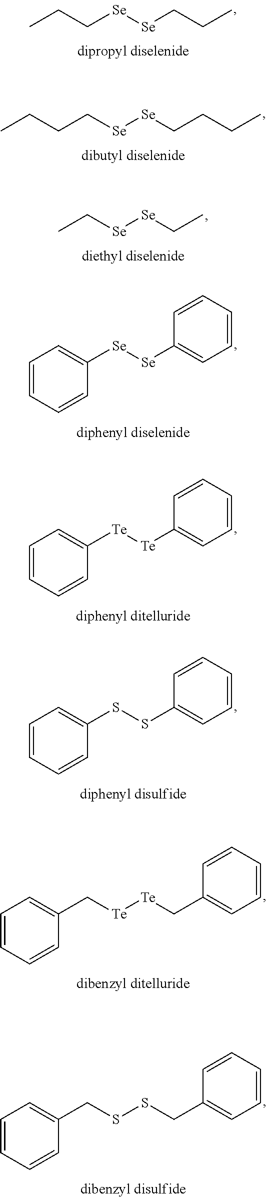 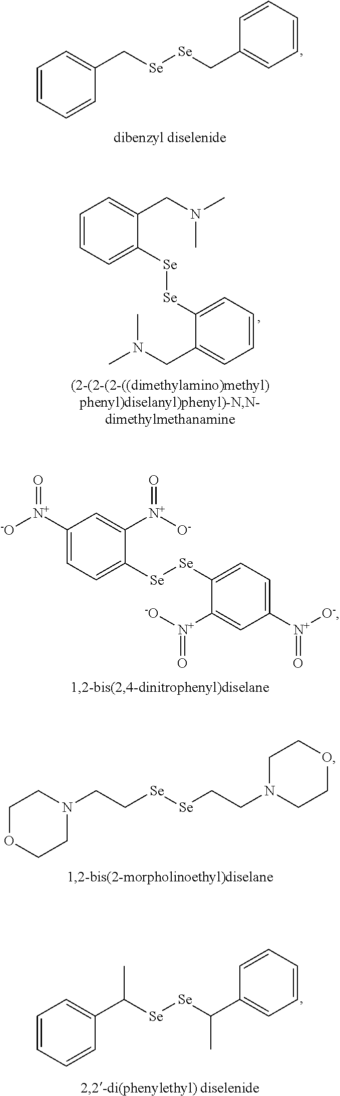 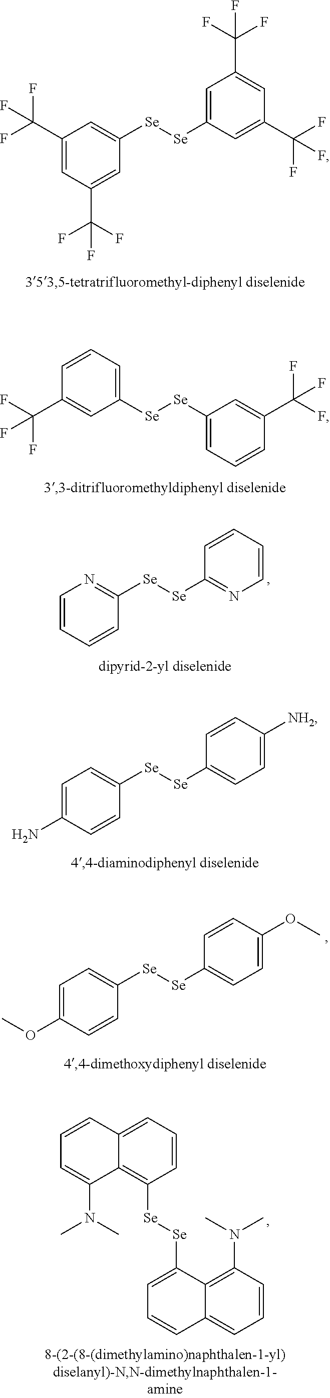 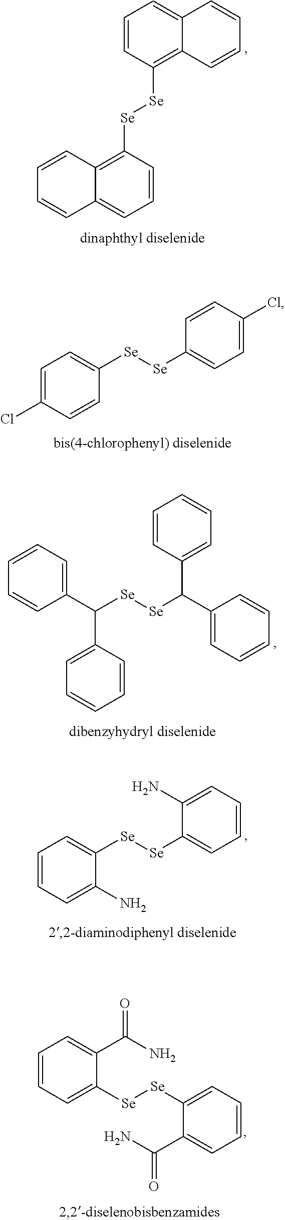 [SMILES: c1ccc2c([Se][Se]c3cccc4ccccc34)cccc2c1 ; Clc1ccc([Se][Se]c2ccc(Cl)cc2)cc1 ; c1ccc(C([Se][Se]C(c2ccccc2)c2ccccc2)c2ccccc2)cc1 ; Nc1ccccc1[Se][Se]c1ccccc1N ; NC(=O)c1ccccc1[Se][Se]c1ccccc1C(N)=O] 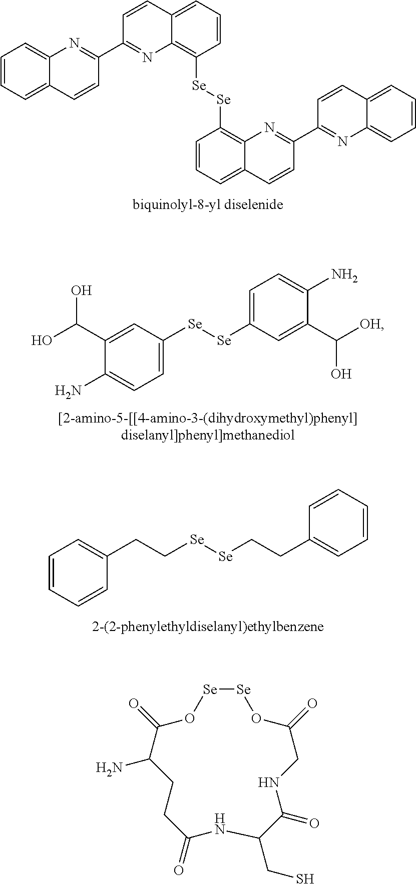 [SMILES: c1ccc2nc(-c3ccc4cccc([Se][Se]c5cccc6ccc(-c7ccc8ccccc8n7)nc56)c4n3)ccc2c1 ; Nc1ccc([Se][Se]c2ccc(N)c(C(O)O)c2)cc1C(O)O ; c1ccc(CC[Se][Se]CCc2ccccc2)cc1 ; NC1CCC(=O)NC(CS)C(=O)NCC(=O)O[Se][Se]OC1=O]
14-amino-9-(sulfanylmethyl)-1,4-dioxa-2,3-diselena-7,10-diazacyclopentadecane-5,8,11,15-tetrone a pharmaceutically acceptable salt thereof, or a combination thereof.

14. (canceled)

15. The pharmaceutical composition of claim 1, wherein the one or more cytotoxicity enhancing agent(s) is a synthetic RNA encoding human SLC7A11 gene.

16. The pharmaceutical composition of claim 1, wherein the one or more cytotoxicity enhancing agent(s) is a cystine/glutamate transporter protein encoded by human SLC7A11 gene.

17. A method for treating cancer comprising administering to a subject in need thereof a therapeutically effective amount of the pharmaceutical composition of claim 1.

18. (canceled)

19. (canceled)

20. (canceled)

21. A pharmaceutical composition comprising:
one or more compounds having Formula II
R₁—X—R₂, Formula II,
a pharmaceutically acceptable salt thereof,
wherein
R₁ is hydrogen, substitute or unsubstituted alkyl or aryl, or —Y—R₃;
R₂ is substituted or unsubstituted alkyl, aryl, heteroaryl, heteroalkyl, —Y—R₃, or a combination thereof;
X is a S, Se, or Te;
Y is a S, Se, or Te;
R₃ is substituted or unsubstituted alkyl, aryl, heteroaryl, heteroalkyl, or a combination thereof,
wherein when present the alkyl is optionally substituted with an alkyl, aryl, primary amine, secondary amine, tertiary amine, primary amide, secondary amide, tertiary amide, carboxylic acid, thiol, carbonyl, hydroxy, ether, ester, halogen or a combination thereof;
wherein when present the aryl is optionally substituted alkyl, aryl, primary amine, secondary amine, tertiary amine, primary amide, secondary amide, tertiary amide, carboxylic acid, thiol, carbonyl, hydroxy, ether, ester, halogen, —CF3 or a combination thereof;
a compound selected from sodium selenite, selenium dioxide, sodium selenide, selenious acid or a combination thereof; and
a pharmaceutically acceptable carrier.

22-30. (canceled)

31. A method of inhibiting main viral protease comprising
administering a pharmaceutical composition of claim 21 to a subject.

32. A method of treating a coronavirus infection, Zika virus, rhinovirus, influenza virus infection, human immunodeficiency virus (HIV), or cutaneous leishmaniasis comprising administering a composition of claim 21 to a subject.

33. A method of treating a hyperinflammation arising out of other viral or bacterial infections comprising administering a composition of claim 21 to a subject.

34-35. (canceled)
